(19) United States
(12) Patent Application Publication
(10) Pub. No.: US 2021/0317502 A1
(43) Pub. Date: Oct. 14, 2021

(54) Genetically-Modified Bacteria And Uses Thereof

(71) Applicants: Cambrex Karlskoga AB, Karlskoga (SE); Rijksuniversiteit Groningen, Groningen (NL)

(72) Inventors: Jonathan Knight, Karlskoga (SE); Cecilia Kvarnstrom Branneby, Karlskoga (SE); Lubbert Dijkhuizen, Groningen (NL); Janet Maria Petrusma, Groningen (NL); Laura Fernandez De Las Heras, Groningen (NL)

(21) Appl. No.: 17/266,214

(22) PCT Filed: Aug. 9, 2019
(86) PCT No.: PCT/EP2019/071468

§ 371 Date: Feb. 5, 2021

(30) Foreign Application Priority Data
Aug. 9, 2018 (GB) 1812997.3

(51) Int. Cl. C12P 33/00; C12N 15/10; C12N 1/20

(52) U.S. Cl. CPC C12P 33/00; C12R 2001/06; C12N 1/20; C12N 15/102

(57) ABSTRACT

A genetically-modified bacterium, for example of the class Actinobacteria, and the use of such a bacterium in the bioconversion of a steroidal substrate into a steroidal product of interest. A method of converting a steroidal substrate into a steroidal product of interest, wherein the method comprises: inoculating culture medium with genetically-modified bacteria according to any of Claims 1 to 28 and growing the bacterial culture until a target OD₆₀₀ is reached; adding a steroidal substrate to the bacterial culture when the target OD₆₀₀ is reached; culturing the bacterial culture so that the steroidal substrate is converted to the steroidal product of interest; and extracting and/or purifying the steroidal product of interest from the bacterial culture.

The present invention relates to genetically-modified bacteria and the use of such bacteria in the bioconversion of steroidal substrates into steroidal compounds of interest. The genetically-modified bacteria may be from the genera Rhodococcus or Mycobacterium.

Steroids are a large and diverse class of organic compounds, with many essential functions in eukaryotic organisms. For example, naturally occurring steroids are involved in maintaining cell membrane fluidity, controlling functions of the male and female reproductive systems and modulating inflammation.

As signalling through steroid controlled pathways is important in a wide variety of processes, the ability to modulate these pathways using synthetically produced steroid drugs means they are an important class of pharmaceuticals. For example, corticosteroids are used as anti-inflammatories for the treatment of conditions such as asthma and rheumatoid arthritis, synthetic steroid hormones are widely used as hormonal contraceptives and anabolic steroids can be used to increase muscle mass and athletic performance.

The synthesis of steroids for use as pharmaceuticals involves either semi-synthesis from natural sterol precursors or total synthesis from simpler organic molecules. Semi-synthesis from sterol precursors such as cholesterol often involves the use of bacteria. The advantages of using bacteria to carry out these bioconversions are that the synthesis involves less steps and the reactions performed by the enzymes are stereospecific, resulting in the production of the desired isomers without the need for protection and deprotection used in traditional chemical synthesis. The products of bacterial bioconversions can then be used as pharmaceuticals or as precursors for further chemical modification to produce the compound of interest.

Steroids naturally occur in both plant, animal and fungal species, and are produced by certain species of bacteria. Despite them only occurring naturally in only a few bacterial species, several bacterial species are able to metabolise sterol compounds as growth substrates. Examples of bacteria that can degrade sterol compounds include those from the genera Rhodococcus and Mycobacterium.

The bacterial sterol metabolism pathway involves progressive oxidation of the sterol side-chain, and breakdown of the polycyclic ring system. The pathway of sterol side-chain degradation in Rhodococcus has been previously investigated using mutant strains (Wilbrink et al, 2011. Applied and Environmental Microbiology, 77(13):4455-4464) and an overview of the cholesterol catabolic pathway is shown in FIG. 1. It has now been found that bacterial species may be used for steroid compound production by genetic modification to block the degradation pathway prior to breakdown of the polycyclic ring system and at various points in side-chain oxidation to allow accumulation of the steroidal compounds of interest in order to improve the yields obtained.

In a first aspect, the invention provides a genetically-modified bacterium blocked in the steroid metabolism pathway prior to degradation of the polycyclic steroid ring system, wherein the bacterium is disrupted in the steroid side-chain degradation pathway, and wherein the bacterium converts a steroidal substrate into a steroidal product of interest.

By “steroid” or “steroidal” compounds we include the meaning of a class of natural or synthetic organic compounds derived from the steroid core structure represented below (with IUPAC-approved ring lettering and atom numbering):

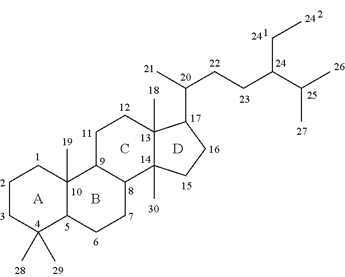

Steroidal compounds generally comprise four fused rings (three six-member cyclohexane rings (rings A, B and C above) and one five-member cyclopentane ring (ring D above)) but vary by the functional groups attached to that four-ring core and by the oxidation state of the rings. For example, sterols are a sub-group of steroidal compounds where one of the defining features is the presence of a hydroxy group (OH) at position 3 or the structure shown above. The structure formed by the atoms labelled 20 to 27 (including positions 24¹ and 24²) in the above diagram is referred to as the steroid side-chain. Non-limiting examples of steroids include: sterols, 3-oxo-4-cholenic acid, 3-oxo-chola-4,22-dien-24-oic acid, 3-oxo-7-hydroxy-4-cholenic acid, 3-oxo-9-hydroxy-4-cholenic acid, 3-oxo-7,9-dihydroxy-4-cholenic acid, 3-oxo-23,24-bisnor-4-cholene-22-oic acid (4-BNC), 3-oxo-23,24-bisnor-1,4-choladiene-22-oic acid (1,4-BNC), 4-androstene-3,17-dione (AD), 1,4-androstadiene-3,17-dione (ADD), sex steroids (e.g. progesterone, testosterone, estradiol), corticosteroids (e.g. cortisol), neurosteroids (e.g. DHEA and allopregnanolone), and secosteroids (e.g. ergocalciferol, cholecalciferol, and calcitriol). Non-limiting examples of steroidal compounds are also shown in FIG. 4.

By “disrupted in the steroid side-chain degradation pathway” we include the meaning of a bacterium in which the normal degradation of the steroid side-chain is impaired. Normally, degradation of the steroid side-chain involves the initial cycle of side-chain activation followed by three successive cycles of β-oxidation (i.e. first, second, and third cycles of β-oxidation). In an unimpaired side-chain degradation pathway, the final product of the side-chain degradation steps is usually 4-androstene-3,17-dione (AD). Thus, a bacterium disrupted in the steroid side-chain degradation pathway will accumulate steroidal products that are upstream of the production of AD. The suggested side-chain degradation pathways of the sterols cholesterol and β-sitosterol are shown in FIG. 2 and FIG. 3 respectively (Wilbrink, 2011. Microbial sterol side chain degradation in Actinobacteria. Thesis).

By “polycyclic steroid ring system” we include the meaning of the ABCD system of rings found in the core steroidal structure shown above in the definition of steroidal.

In some embodiments, the disruption in the steroid side-chain degradation pathway occurs after the first cycle of β-oxidation.

By “first cycle of β-oxidation” we include the meaning of the first cycle of β-oxidation in the steroid side-chain degradation pathway (Wipperman et al, 2014. Crit. Rev. Biochem. Mol. Biol., 49(4):269-293). Specifically, the first cycle of β-oxidation is the process immediately following the side-chain activation cycle step, resulting in the shortening of the side-chain and the production of a C₂₄ steroidal compound.

In some embodiments, the steroidal substrate may be a sterol substrate. In certain embodiments, the sterol substrate may comprise:

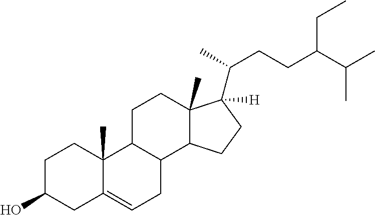 [SMILES: CCC(CC[C@@H](C)[C@H]1CCC2C3CC=C4C[C@@H](O)CC[C@]4(C)C3CC[C@@]21C)C(C)C]

β-sitosterol;

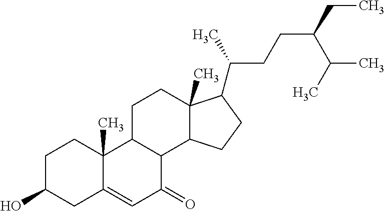 [SMILES: CC[C@H](CC[C@@H](C)C1CCC2C3C(=O)C=C4C[C@@H](O)CC[C@]4(C)C3CC[C@@]21C)C(C)C]

7-oxo-β-sitosterol or 7-hydroxy-β-sitosterol;

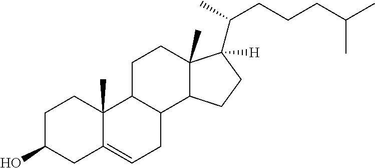 [SMILES: CC(C)CCC[C@@H](C)[C@H]1CCC2C3CC=C4C[C@@H](O)CC[C@]4(C)C3CC[C@@]21C]

cholesterol;

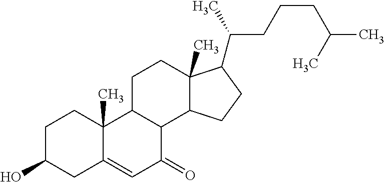 [SMILES: CC(C)CCC[C@@H](C)C1CCC2C3C(=O)C=C4C[C@@H](O)CC[C@]4(C)C3CC[C@@]21C]

7-oxo-cholesterol or 7-hydroxy-β-cholesterol;

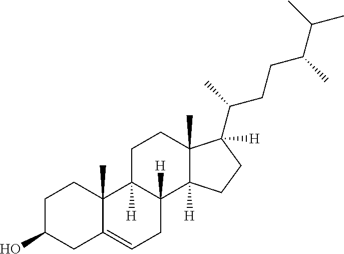

campesterol;

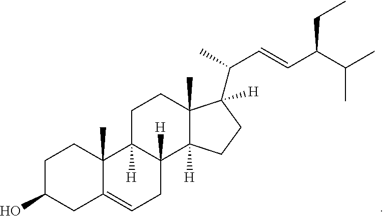

stigmasterol;

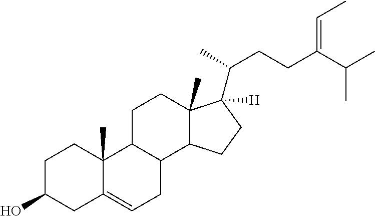 [SMILES: C/C=C(/CC[C@@H](C)[C@H]1CCC2C3CC=C4C[C@@H](O)CC[C@]4(C)C3CC[C@@]21C)C(C)C]

fucosterol;
7-oxo-phytosterol; or a combination thereof.

By “sterol” we include the meaning of molecules belonging to a class of lipids which are a sub-group of steroids with a hydroxyl group at the 3-position of the A-ring. Sterols have the general structure:

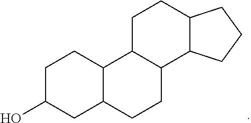 [SMILES: OC1CCC2C(CCC3C4CCCC4CCC23)C1]

Sterols may also be referred to as steroid alcohols, and occur naturally in plants (phytosterols), animals (zoosterols), and fungi, and can be also produced by some bacteria. Non-limiting examples of sterols include: β-sitosterol, 7-oxo-β-sitosterol, 7-hydroxy-β-sitosterol, cholesterol, 7-oxo-cholesterol, 7-hydroxy-β-cholesterol, campesterol, stigmasterol, fucosterol, 7-oxo-phytosterol, adosterol, atheronals, avenasterol, azacosterol, cerevisterol, colestolone, cycloartenol, 7-dehydrocholesterol, 5-dehydroepisterol, 7-dehydrositosterol, 20a,22R-dihydroxycholesterol, dinosterol, epibrassicasterol, episterol, ergosterol, ergosterol peroxide, fecosterol, fucosterol, fungisterol, ganoderiol, ganodermadiol, 7α-hydroxycholesterol, 22R-hydroxycholesterol, 27-hydroxycholesterol, inotodiol, lanosterol, lathosterol, lichesterol, lucidadiol, lumisterol, oxycholesterol, oxysterol, parkeol, spinasterol, trametenolic acid, and zymosterol. Non-limiting examples of sterols are also shown in FIG. 5.

In some embodiments, the steroidal product, of interest comprises an intact polycyclic ring system.

By “intact polycyclic ring system” we include the meaning of a steroidal molecule in which the ABCD ring system of the core steroid structure is still present, i.e. the ABCD ring system has not undergone degradation and/or oxidation such that any of the rings have been opened or removed.

In some embodiments, the steroidal product of interest is a steroidal compound with a side-chain having a backbone of five carbons.

By “backbone” we include the meaning of the longest consecutive chain of carbon atoms in the steroid side-chain being five carbon atoms in length. Generally, the five carbons in the backbone are those at positions 20, 21, 22, 23, and 24, as shown in the diagram of the steroid core structure in the definition of the term “steroidal” above.

In certain embodiments, the steroidal product of interest may be:

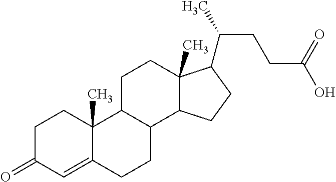 [SMILES: C[C@H](CCC(=O)O)C1CCC2C3CCC4=CC(=O)CC[C@]4(C)C3CC[C@@]21C]

3-oxo-4-cholenic acid;

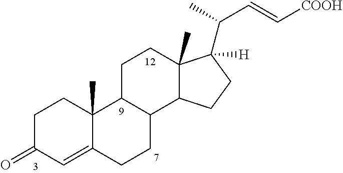 [SMILES: C[C@H](/C=C/C(=O)O)[C@H]1CCC2C3CCC4=CC(=O)CC[C@]4(C)C3CC[C@@]21C]

Chola 4,22-dien-24-oic acid, 3-oxo (CAS 59648-73-6, or CAS 82637-22-7 for pure E isomer);

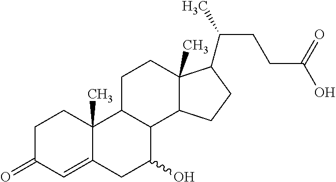 [SMILES: C[C@H](CCC(=O)O)C1CCC2C3C(O)CC4=CC(=O)CC[C@]4(C)C3CC[C@@]21C]

3-oxo-7-hydroxy-4-cholenic acid;

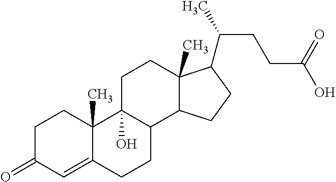

3-oxo-9-hydroxy-4-cholenic acid;

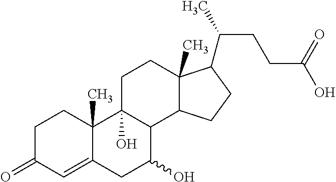

3-oxo-7,9-dihydroxy-4-cholenic acid;

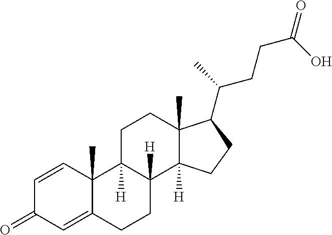

3-oxo-1,4-choladienoic acid;

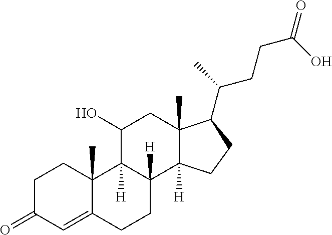

3-oxo-11-hydroxy-4-cholenic acid;

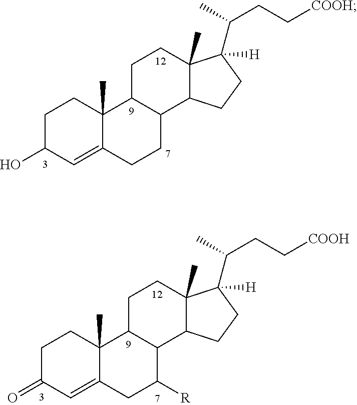

wherein R can be hydroxyl, oxo, or a halogen;

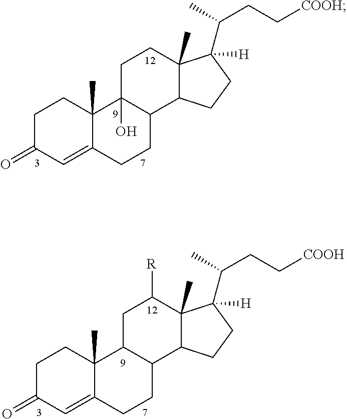

wherein R can be hydroxyl or oxo;

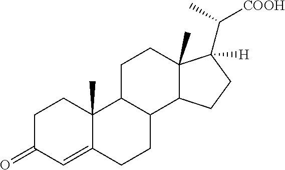 [SMILES: C[C@H](C(=O)O)[C@H]1CCC2C3CCC4=CC(=O)CC[C@]4(C)C3CC[C@@]21C]

3-oxo-23,24-bisnor-4-cholene-22-oic acid (4-BNC);

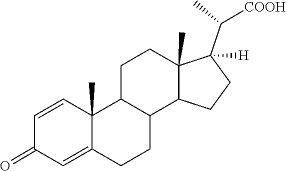 [SMILES: C[C@H](C(=O)O)[C@H]1CCC2C3CCC4=CC(=O)C=C[C@]4(C)C3CC[C@@]21C]

3-oxo-23,24-bisnor-1,4-choladiene-22-oic acid (1,4-BNC); or variants thereof.

In other preferred embodiments, the steroidal product of interest may be

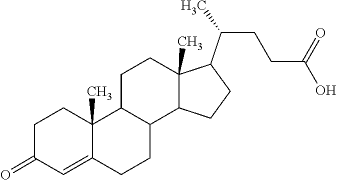 [SMILES: C[C@H](CCC(=O)O)C1CCC2C3CCC4=CC(=O)CC[C@]4(C)C3CC[C@@]21C]

3-oxo-4-cholenic acid;

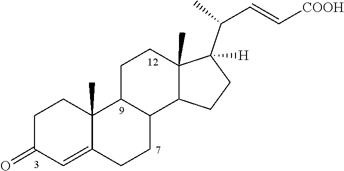 [SMILES: C[C@H](/C=C/C(=O)O)[C@H]1CCC2C3CCC4=CC(=O)CC[C@]4(C)C3CC[C@@]21C]

Chola 4,22-dien-24-oic acid, 3-oxo (CAS 59648-7-6, or CAS 82637-22-7 for pure E isomer);

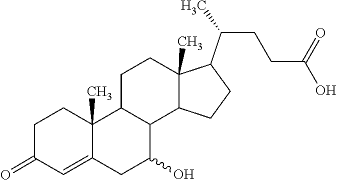 [SMILES: C[C@H](CCC(=O)O)C1CCC2C3C(O)CC4=CC(=O)CC[C@]4(C)C3CC[C@@]21C]

3-oxo-7-hydroxy-4-cholenic acid;

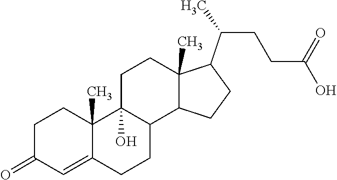

3-oxo-9-hydroxy-4-cholenic acid;

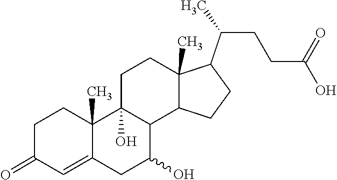

3-oxo-7,9-dihydroxy-4-cholenic acid;

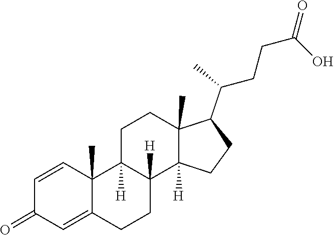

3-oxo-1,4-choladienoic acid;

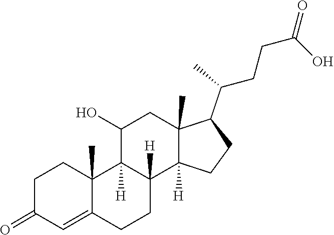

3-oxo-11-hydroxy-4-cholenic acid;

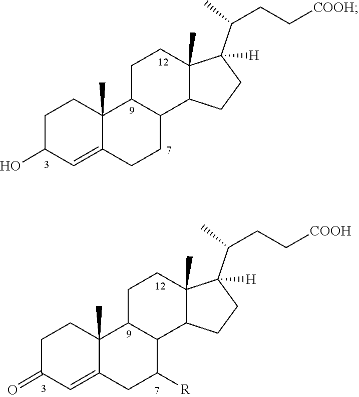

wherein R can be hydroxyl, oxo, or a halogen;

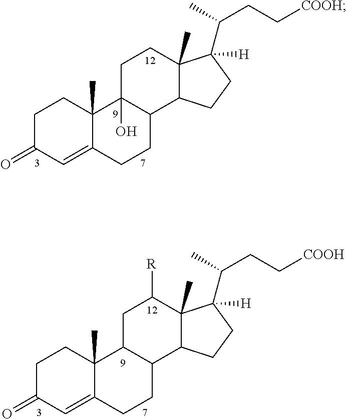

wherein R can be hydroxyl or oxo; or variants thereof.

In some embodiments, the genetically-modified bacterium may be of the Actinobacteria class or the Gammaproteobacteria class.

In certain embodiments, a genetically modified bacterium of the Actinobacteria class may be a Rhodococcus species, a Mycobacterium species, a Nocardia species, a Corynebacterium species, or an Arthrobacter species.

Where the bacterium is of a Rhodococcus species, the Rhodococcus species may be Rhodococcus rhodochrous, Rhodococcus erythropolis, Rhodococcus jostii, Rhodococcus ruber, preferably Rhodococcus rhodochrous.

Where the bacterium is of a Mycobacterium species, the Mycobacterium species may be Mycobacterium neoaurum, Mycobacterium smegmatis, Mycobacterium tuberculosis, or Mycobacterium fortuitum, preferably Mycobacterium neoaurum.

Where the bacterium is of a Nocardia species, the Nocardia species may be Nocardia restrictus, Nocardia corallina, or Nocardia opaca.

Where the bacterium is of a Arthrobacter species, the Arthrobacter species may be Arthrobacter simplex.

In some embodiments, the genetically-modified bacterium comprises one or more genetic modifications. In certain embodiments, the genetic modification of the genetically-modified bacterium may comprise inactivation of the genes: kshA1 (SEQ ID NO: 1), kshA2 (SEQ ID NO: 2), kshA3 (SEQ ID NO: 3), kshA4 (SEQ ID NO: 4), kshA5 (SEQ ID NO: 5), or homologs thereof.

By “genetic modification” we include the meaning of an artificial alteration or addition to the genetic material present in an organism. For example, a genetic modification may be a directed deletion of a gene or genomic region, a directed mutagenesis of a gene or genomic region (e.g. a point mutation), the addition of a gene or genetic material to the genome of the organism (e.g. an integration), or, in the case of bacteria, the transformation of such cells with plasmid.

By “homolog” we include the meaning of a second gene or polypeptide that has a similar biological function to a first gene or polypeptide and may also have a degree of sequence similarity to the first gene or polypeptide. A homologous gene may encode a polypeptide that exhibits a degree of sequence similarity to a polypeptide encoded by the corresponding first gene. For example, a homolog may be a similar gene in a different species derived from a common ancestral gene (ortholog), or a homolog may be a second similar gene within the genome of a single species that is derived from a gene duplication event (paralog). A homologous gene or polypeptide may have a nucleotide or amino acid sequence that varies from the nucleotide or amino acid sequence of the first gene or polypeptide, but still maintains functional characteristics associated with the first gene or polypeptide (e.g. in the case where a homologous polypeptide is an enzyme, the homologous polypeptide catalyses the same reaction as the first polypeptide). The variations that can occur in a nucleotide or amino acid sequence of a homolog may be demonstrated by nucleotide or amino acid differences in the overall sequence or by deletions, substitutions, insertions, inversions or additions of nucleotides or amino acids in said sequence.

In some embodiments, the genetic modification further comprises re-introduction of a wild type copy of the kshA5 gene comprising SEQ ID NO: 5, or a homolog thereof.

In other embodiments, the genetic modification comprises inactivation of the genes: kshA1 (SEQ ID NO: 1), kshA2 (SEQ ID NO: 2), kshA3 (SEQ ID NO: 3), and kshA4 (SEQ ID NO: 4), or homologs thereof.

In some embodiments, the genetic modification of the genetically-modified bacterium further comprises inactivation of the genes: kstD1(SEQ ID NO: 6), kstD2 (SEQ ID NO: 7), and kstD3 (SEQ ID NO: 8), or homologs thereof.

In some embodiments, the genetic modification comprises inactivation of one or more of the genes: fadE34 (SEQ ID NO: 9; SEQ ID NO: 12), fadE34#2 (SEQ ID NO: 10), or homologs thereof.

In other preferred embodiments, the genetic modification of the genetically-modified bacterium further comprises inactivation of the gene: fadE26 (SEQ ID NO: 11), or homologs thereof.

In some embodiments, where the genetic modification comprises a gene inactivation, the gene activation is by gene deletion.

By “gene deletion” we include the meaning of removal of all or substantially all of a gene or genomic region from the genome of an organism, such that the functional polypeptide product(s) encoded by that gene or genomic region is no longer produced by the organism.

In certain embodiments, the homolog has a nucleotide sequence with at least 50% sequence identity with the nucleotide sequence of a first gene. In other embodiments, the homolog has a nucleotide sequence that has a sequence identity of at least 55%, at least 60%, at least 65%, at least 70%, at least 75%, at least 80%, at least 85%, at least 90%, at least 91%, at least 92%, at least 93%, at least 94%©, at least 95%, at least 96%, at least 97%©, at least 98%, or at least 99% with the nucleotide sequence of a first gene.

In some embodiments, the homolog encodes a polypeptide that has an amino acid sequence with at, least 50% sequence identity with the amino acid sequence of a first polypeptide. The homolog encodes a polypeptide that has an amino acid sequence identity of at least 55%©, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%.

By “sequence identity” we include the meaning of the extent to which two nucleotide or amino acid sequences are similar, measured in terms of a percentage identity. Optimal alignment is determined by comparing two optimally aligned sequences over a comparison window, where the fragment of the nucleotide or amino acid sequence in the comparison window may comprise additions or deletions (e.g. gaps or overhangs) as compared to the reference sequence (which does not comprise additions or deletions) for optimal alignment of the two sequences. The percentage is calculated by determining the number of positions at which the identical nucleotide base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used.

In certain embodiments, the genetically-modified Rhodococcus rhodochrous bacterium may be of strain: LM9 (Accession No. NCIMB 43058), LM19 (Accession No. NCIMB 43059), or LM33 (Accession No. NCIMB 43060).

In certain embodiments, the genetically-modified Mycobacterium neoaurum bacterium may be of strain: NRRL B-3805 Mneo-ΔfadE34 (Accession No. NCIMB 43057).

In a second aspect, the invention provides a genetically-modified bacterium according to the first aspect for use in the conversion of a steroidal substrate into a steroidal compound of interest.

In a third aspect, the invention provides a method of converting a steroidal substrate into a steroidal product of interest, comprising the steps of:
(a) inoculating culture medium with genetically-modified bacteria according to the first or second aspect and growing the bacterial culture until a target OD₆₀₀ is reached;
(b) adding a steroidal substrate to the bacterial culture when the target OD₆₀₀ is reached;
(c) culturing the bacterial culture so that the steroidal substrate is converted to the steroidal product of interest; and,
(d) extracting and/or purifying the steroidal product of interest from the bacterial culture.

By “culture medium” we include the meaning of a solid, liquid, or semi-solid medium designed to support the growth of microorganisms or cells.

In some embodiments, the culture medium may be Luria-Bertani (LB) medium (10 g/L tryptone; 5 g/L yeast extract; 10 g/L NaCl) or minimal medium (4.65 g/L K₂HPO₄; 1.5 g/L NaH₂PO₄.H₂O; 3 g/L NH₄Cl; 1 g/L MgSO₄.7H₂O; 1 ml/L Vishniac trace element solution).

In certain embodiments, in step (a) of the method the bacterial culture may be grown to a target OD₆₀₀ of at least 0.25, at least 0.5, at least 0.75, at least 1.0, at least 1.5, at least 2.0, at least 2.5, at least 3.0, at least 3.5, at least 4.0, at least 4.1, at least 4.2, at least 4.3, at least 4.4, at least 4.5, at least 4.6, at least 4.7, at least 4.8, at least 4.9, or at least 5.0. Preferably, the target OD₆₀₀ may be at least 1.0, more preferably at least 4.0, yet more preferably at least 4.5, most preferably at least 5.0.

In some embodiments of the method, the steroidal substrate may be a sterol substrate. In certain embodiments, the sterol substrate may comprise:

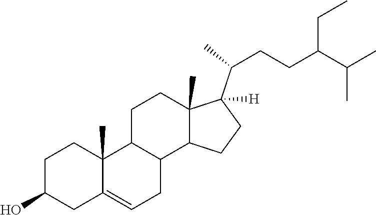 [SMILES: CCC(CC[C@@H](C)[C@H]1CCC2C3CC=C4C[C@@H](O)CC[C@]4(C)C3CC[C@@]21C)C(C)C]

β-sitosterol;

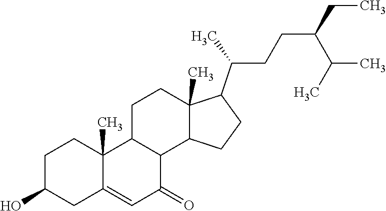 [SMILES: CC[C@H](CC[C@@H](C)C1CCC2C3C(=O)C=C4C[C@@H](O)CC[C@]4(C)C3CC[C@@]21C)C(C)C]

7-oxo-β-sitosterol or 7-hydroxy-β-sitosterol;

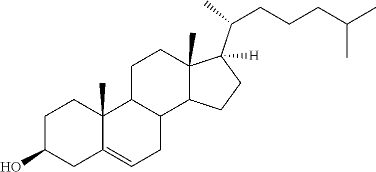 [SMILES: CC(C)CCC[C@@H](C)[C@H]1CCC2C3CC=C4C[C@@H](O)CC[C@]4(C)C3CC[C@@]21C]

cholesterol;

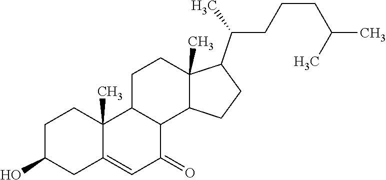 [SMILES: CC(C)CCC[C@@H](C)C1CCC2C3C(=O)C=C4C[C@@H](O)CC[C@]4(C)C3CC[C@@]21C]

7-oxo-cholesterol or 7-hydroxy-β-cholesterol;

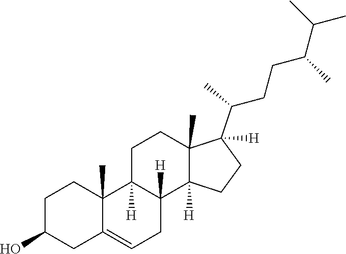

campesterol;

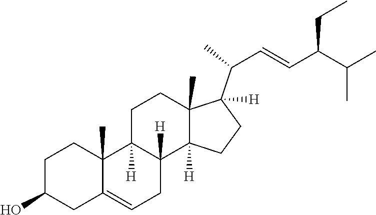

stigmasterol;

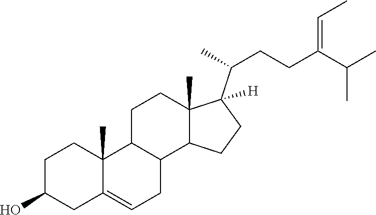 [SMILES: C/C=C(/CC[C@@H](C)[C@H]1CCC2C3CC=C4C[C@@H](O)CC[C@]4(C)C3CC[C@@]21C)C(C)C]

fucosterol;
7-oxo-phytosterol; or a combination thereof.

In some embodiments of the method, the steroidal product of interest may comprise an intact polycyclic ring system. In certain embodiments, the steroidal product of interest may be a steroidal compound with a side-chain having a backbone of five carbons.

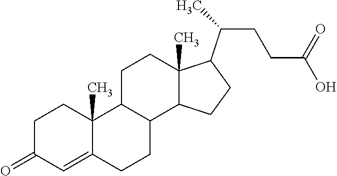 [SMILES: C[C@H](CCC(=O)O)C1CCC2C3CCC4=CC(=O)CC[C@]4(C)C3CC[C@@]21C]

3-oxo-4-cholenic acid;

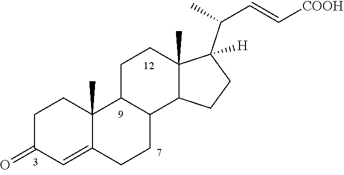 [SMILES: C[C@H](/C=C/C(=O)O)[C@H]1CCC2C3CCC4=CC(=O)CC[C@]4(C)C3CC[C@@]21C]

Chola 4,22-dien-24-oic acid, 3-oxo (CAS 59648-73-6, or CAS 82637-22-7 for pure E isomer);

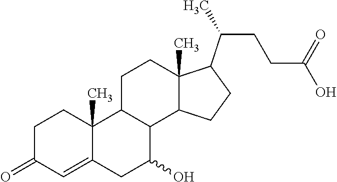 [SMILES: C[C@H](CCC(=O)O)C1CCC2C3C(O)CC4=CC(=O)CC[C@]4(C)C3CC[C@@]21C]

3-oxo-7-hydroxy-4-cholenic acid;

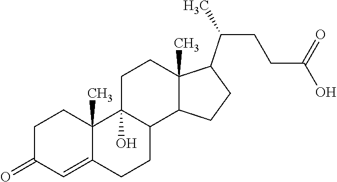

3-oxo-9-hydroxy-4-cholenic acid;

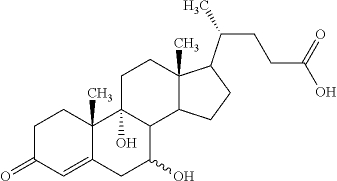

3-oxo-7,9-dihydroxy-4-cholenic acid;

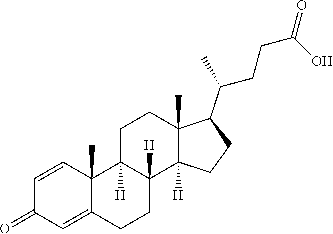

3-oxo-1,4-choladienoic acid;

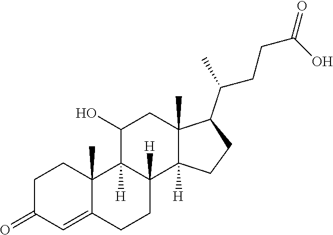

3-oxo-11-hydroxy-4-cholenic acid;

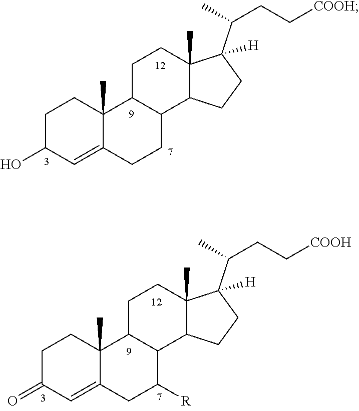

wherein R can be hydroxyl, oxo, or a halogen;

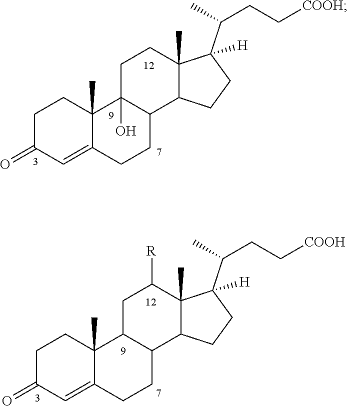

wherein R can be hydroxyl or oxo;

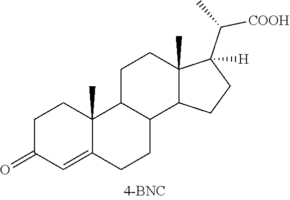 [SMILES: C[C@H](C(=O)O)[C@H]1CCC2C3CCC4=CC(=O)CC[C@]4(C)C3CC[C@@]21C]

3-oxo-23,24-bisnor-4-cholene-22-oic acid (4-BNC);

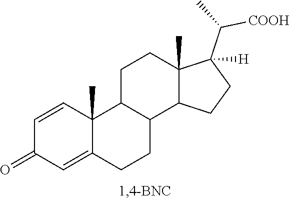 [SMILES: C[C@H](C(=O)O)[C@H]1CCC2C3CCC4=CC(=O)C=C[C@]4(C)C3CC[C@@]21C]

3-oxo-23,24-bisnor-1,4-choladiene-22-oic acid (4-BNC); or variants thereof.

In some preferred embodiments, the steroidal product of interest may be:

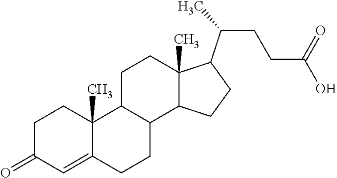 [SMILES: C[C@H](CCC(=O)O)C1CCC2C3CCC4=CC(=O)CC[C@]4(C)C3CC[C@@]21C]

3-oxo-4-cholenic acid;

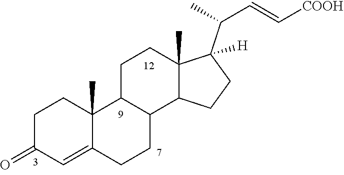 [SMILES: C[C@H](/C=C/C(=O)O)[C@H]1CCC2C3CCC4=CC(=O)CC[C@]4(C)C3CC[C@@]21C]

Chola 4,22-dien-24-oic acid, 3-oxo (CAS 59648-73-6, or CAS 82637-22-7 for pure E isomer);

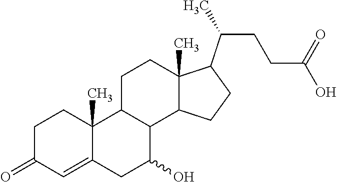 [SMILES: C[C@H](CCC(=O)O)C1CCC2C3C(O)CC4=CC(=O)CC[C@]4(C)C3CC[C@@]21C]

3-oxo-7-hydroxy-4-cholenic acid;

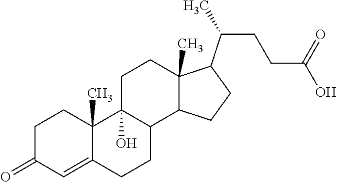

3-oxo-9-hydroxy-4-cholenic acid;

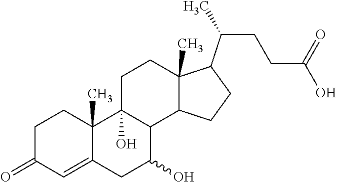

3-oxo-7,9-dihydroxy-4-cholenic acid;

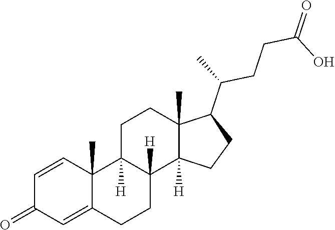

3-oxo-1,4-choladienoic acid;

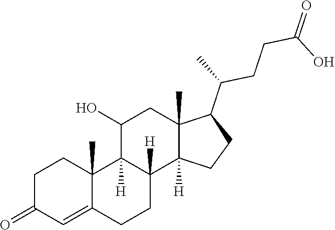

3-oxo-11-hydroxy-4-cholenic acid;

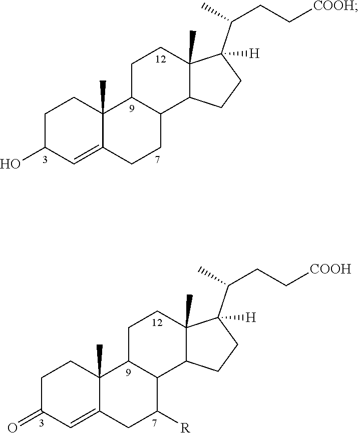

wherein R can be hydroxyl, oxo, or a halogen;

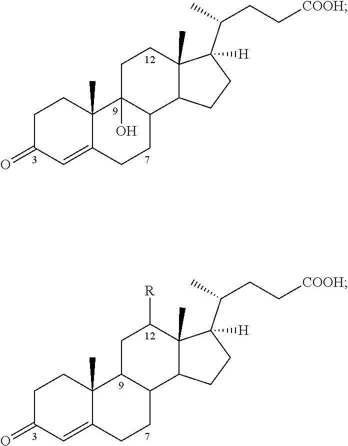

or variants thereof.

In some embodiments, in step (b) of the method, the steroidal substrate may be added at a concentration of at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. Preferably, the steroidal substrate may be added at a concentration of at east 1 mM, more preferably at least 1.5 mM, most preferably at least 2.0 mM.

In some embodiments, in step (b) of the method a cyclodextrin may be added to the culture medium.

By “cyclodextrin” we include the meaning of a compound made up of sugar molecules bound together in a ring, where the ring is composed of 5 or more α-D-glucopyranoside units linked 1→4. Non-limiting examples of cyclodextrins include: α-cyclodextrin, β-cyclodextrin, γ-cyclodextrin, methyl-β-cyclodextrin, and 2-OH-propyl-β-cyclodextrin.

In certain embodiments, the cyclodextrin may be a β-cyclodextrin or a γ-cyclodextrin. Where the cyclodextrin is a β-cyclodextrin, it may be a methyl-β-cyclodextrin or a 2-OH-propyl-β-cyclodextrin.

In some embodiments, the cyclodextrin is added at a concentration of 1 mM to 50 mM, 2 mM to 45 mM, 3 mM to 40 mM, 4 mM to 35 mM, 5 mM to 30 mM, 6 mM to 29 mM, 7 mM to 28 mM, 8 mM to 27 mM, 9 mM to 26 mM, 10 mM to 25 mM, 11 mM to 24 mM, 12 mM to 23 mM, 13 mM to 22 mM, 14 mM to 21 mM, 15 mM to 21 mM, 16 mM to 20 mM, 17 mM to 19 mM, 1 mM to 18 mM. Preferably, the cyclodextrin may be added at a concentration of 1 mM to 25 mM, more preferably 5 mM to 25 mM.

In other embodiments, the cyclodextrin is added at a concentration of at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, at least 20 mM, at least 21 mM, at least 22 mM, at least 23 mM, at least 24 mM, at least 25 mM, at least 30 mM, at least 35 mM, at least 40 mM, at least 45 mM, or at least 50 mM. Preferably the cyclodextrin is added at a concentration of at least 1 mM, preferably at least 5 mM, more preferably at least 12.5 mM, most preferably at least 25 mM.

In some embodiments, in step (b) of the method an organic solvent may be added to the culture medium.

By “organic solvent” we include the meaning of a carbon-based solvent capable of dissolving other substances. Non-limiting examples of organic solvents include: ethanol, dimethylformamide(DMF), acetone, methanol, isopropanol, dimethyl sulfoxide (DMSO), and toluene.

In certain embodiments, the organic solvent may be ethanol, dimethylformamide (DMF), or acetone. Preferably, the organic solvent may be ethanol.

In some embodiments, the organic solvent is added the culture medium at a volume/volume (v/v) concentration of 1% to 20%©, 2% to 19%, 3%©, to 18%, 4% to 17%, 5% to 16%, 6% to 15%, 7% to 14%, 8%, to 13%, 9% to 12%, 10% to 11%. Preferably, the organic solvent may be added at a volume/volume (v/v) concentration of 5% to 20%, more preferably 5% to 15%.

In some embodiments, the organic solvent is added to the culture medium at a volume/volume (v/v) concentration of at least 1%, at least 2%, at least 3%, at least 4%, at least 5%, at least 6%, at least 7%, at least 8%, at least 9%, at least 10%©, at least 11%, at least 12%, at least 13%, at least 14%, at least 15%, at least 16%, at least 17%, at least 18%, at least 19%, or at least 20%©. Preferably, the organic solvent may be added at a volume/volume (v/v) concentration of at least 1%. More preferably, the organic solvent may be added at a volume/volume (v/v) concentration of at least 5%.

In some embodiments, in step (b) of the method a cyclodextrin and an organic solvent are added to the culture medium.

In certain embodiments, where a cyclodextrin and an organic solvent are added to the culture medium, the cyclodextrin is added at a concentration of 1 mM to 25 mM, 2 mM to 24 mM, 3 mM to 23 mM, 4 mM to 22 mM, 5 mM to 21 mM, 6 mM to 20 mM, 7 mM to 19 mM, 8 mM to 18 mM, 9 mM to 17 mM, 10 mM to 16 mM, 11 mM to 15 mM, 12 mM to 14 mM, 1 mM to 13 mM, and the organic solvent is added at a volume/volume (v/v) concentration of 1% to 20%©, 2% to 19%©, 3%©, to 18%, 4% to 17%, 5% to 16%, 6% to 15%, 7% to 14%, 8%, to 13%, 9% to 12%©, 10% to 11%. Preferably, the cyclodextrin may be added at concentration of 1 mM to 25 mM and the organic solvent may be added at a volume/volume (v/v) concentration of 1% to 10%. More preferably, the cyclodextrin may be added at concentration of 1 mM to 10 mM and the organic solvent may be added at a volume/volume (v/v) concentration of 1% to 10%. Yet more preferably, the cyclodextrin may be added at concentration of 1 mM to 5 mM and the organic solvent may be added at a volume/volume (v/v) concentration of 1% to 5% Most preferably, the cyclodextrin may be added at concentration of 5 mM and the organic solvent may be added at a volume/volume (v/v) concentration of 5%.

In other embodiments, where a cyclodextrin and an organic solvent are added to the culture medium, the cyclodextrin is added at a concentration of at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, at least 20 mM, at least 21 mM, at least 22 mM, at least 23 mM, at least 24 mM, at least 25 mM, and the organic solvent is added at a volume/volume (v/v) concentration of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%. Preferably, the cyclodextrin may be added at concentration of at least 1 mM and the organic solvent may be added at a volume/volume (v/v) concentration of at least 1%. More preferably, the cyclodextrin may be added at concentration of at least 5 mM and the organic solvent may be added at a volume/volume (v/v) concentration of 5%.

In a fourth aspect, the invention provides a steroidal product of interest produced by the method of the third aspect.

In a fifth aspect, the invention provides a kit for converting a steroidal substrate into a steroidal product of interest, wherein the kit comprises:
(a) a genetically-modified bacterium according to the first aspect; and,
(b) instructions for using the kit.

The kit may further comprise a steroidal substrate.

In some embodiments, the steroidal substrate may be a sterol substrate. In certain embodiments, the sterol substrate comprises:

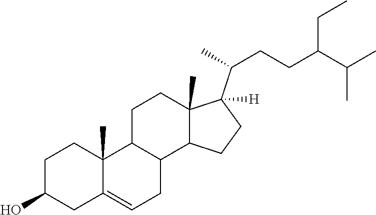 [SMILES: CCC(CC[C@@H](C)[C@H]1CCC2C3CC=C4C[C@@H](O)CC[C@]4(C)C3CC[C@@]21C)C(C)C]

β-sitosterol;

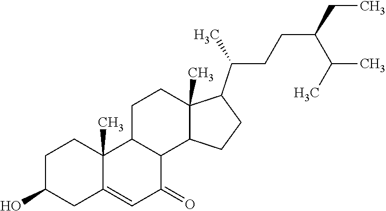 [SMILES: CC[C@H](CC[C@@H](C)C1CCC2C3C(=O)C=C4C[C@@H](O)CC[C@]4(C)C3CC[C@@]21C)C(C)C]

7-oxo-β-sitosterol or 7-hydroxy-β-sitosterol;

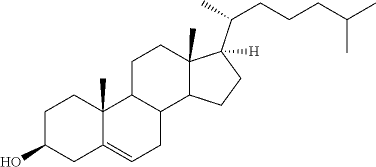 [SMILES: CC(C)CCC[C@@H](C)[C@H]1CCC2C3CC=C4C[C@@H](O)CC[C@]4(C)C3CC[C@@]21C]

cholesterol;

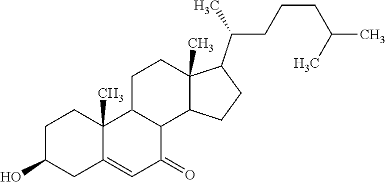 [SMILES: CC(C)CCC[C@@H](C)C1CCC2C3C(=O)C=C4C[C@@H](O)CC[C@]4(C)C3CC[C@@]21C]

7-oxo-cholesterol or 7-hydroxy-β-cholesterol;

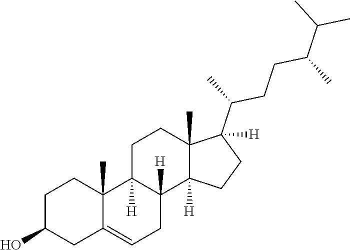

campesterol;

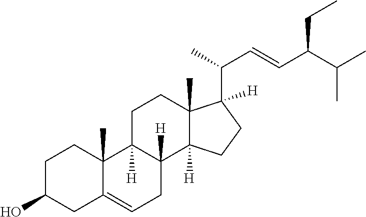

stigmasterol;

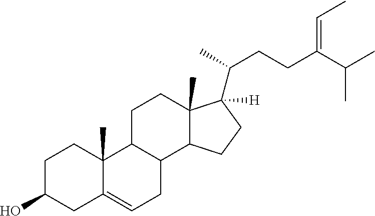 [SMILES: C/C=C(/CC[C@@H](C)[C@H]1CCC2C3CC=C4C[C@@H](O)CC[C@]4(C)C3CC[C@@]21C)C(C)C]

fucosterol;
7-oxo-phytosterol; or a combination thereof.

In some embodiments, the kit may further comprise a cyclodextrin such as a β-cyclodextrin or a γ-cyclodextrin. Preferably, the cyclodextrin is a β-cyclodextrin, more preferably a methyl-β-cyclodextrin or a 2-OH-propyl-β-cyclodextrin.

In some embodiments, the kit may further comprise an organic solvent. In certain embodiments, the organic solvent is ethanol, dimethylformamide (DMF), or acetone, preferably ethanol.

The listing or discussion of an apparently prior-published document in this specification should not necessarily be taken as an acknowledgement that the document is part of the state of the art or is common general knowledge.

The deposits referred to in this disclosure (Accession Nos. NCIMB 43057, NCIMB 43058, NCIMB 43059, and NCIMB 43060) were deposited at the National Collection of Industrial, Food and Marine Bacteria, Ferguson Building, Craibstone Estate, Bucksburn, Aberdeen, AB21 9YA, UK by Cambrex Karlskoga AB on 29 May 2018.

The present invention will now be described in more detail with reference to the following non-limiting figures and examples.

### DESCRIPTION OF THE FIGURES

FIG. 1. Overview of cholesterol catabolic pathway.

FIG. 2. Overview of cholesterol side-chain degradation pathway.

FIG. 3. Overview of β-sitosterol side-chain degradation pathway.

FIG. 4. Examples of steroidal compounds

FIG. 5. Examples of steroidal substrates.

FIG. 6. Total ion chromatogram obtained by LC-MS for LM3 cultured when cholesterol is the starting substrate. Peaks at 7.67 minutes and 8.25 minutes indicate accumulation of 4-androstene-3,17-dione (AD) and 3-oxo-23,24-bisnor-4-cholene-22-oic acid (4-BNC) respectively. NL: Normalisation Level=base peak intensity.

FIG. 7. Product ion mass spectra obtained by LC-MS for LM9 when cholesterol is the starting substrate. (A) Peak at Peak at m/z of 345,24 (positive mode) corresponds to 4-BNC being accumulated when cholesterol is the starting substrate. (B) Peak at m/z of 373.27 (positive mode) corresponds to 3-oxo-4-cholenic acid being accumulated when cholesterol is the starting substrate. NL: Normalisation Level=base peak intensity.

FIG. 8. Product ion mass spectra obtained by LC-MS for LM9 when cholesterol, β-sitosterol, or 7-oxo-sterol is the starting substrate. (A, top) Peak at m/z of 389.27 (positive mode) corresponds to production of 3-oxo-7-hydroxy-4-cholenic acid when 7-oxo-sterol is the starting substrate. (B, middle) Peak at m/z of 373.27 (positive mode) corresponds to production of 3-oxo-4-cholenic acid when β-sitosterol is the starting substrate. (C, bottom) Peak at m/z of 373.27 (positive mode) corresponds to production of 3-oxo-4-cholenic acid when cholesterol is the starting substrate. NL: Normalisation Level=base peak intensity.

FIG. 9. Extracted ion chromatograms obtained by LC-MS for LM19 and LM9 when cholesterol or β-sitosterol is the starting substrate. (A) Strain=LM9; Substrate=Cholesterol. Peak at 9.70 minutes corresponds to production of 3-oxo-4-cholenic acid by LM9. (B) Strain=LM19; Substrate=Cholesterol. Peak at 8.07 minutes corresponds to production of 3-oxo-9-OH-4-cholenic acid by LM19. (C) Strain=LM9; Substrate=β-sitosterol. Peak at 9.68 minutes corresponds to production of 3-oxo-4-cholenic acid by LM9. (D) Strain=LM19; Substrate=β-sitosterol. Peak at 8.09 minutes corresponds to production of 3-oxo-9-OH-4-cholenic acid by LM19. NL: Normalisation Level=base peak intensity.

FIG. 10. Product ion mass spectra obtained by LC-MS confirming identity of peaks produced by LM9 and LM19 when cholesterol or β-sitosterol is the starting substrate. (A) Strain=LM19; Substrate=Cholesterol or β-sitosterol. Peak at m/z of approximately 389.27 (positive mode) corresponds to production of 3-oxo-9-OH-4-cholenic acid by LM19. (B) Strain=LM9; Substrate=Cholesterol or β-sitosterol. Peak at m/z of 373.27 (positive mode) corresponds to production of 3-oxo-4-cholenic acid by LM9. NL: Normalisation Level=base peak intensity.

FIG. 11. Product ion mass spectra obtained by LC-MS for LM19 when 7-oxo-sterol is the starting substrate. Peak at m/z of 405.26 (positive mode) corresponds to production of 3-oxo-7,9-dihydroxy-4-cholenic acid by LM19. NL: Normalisation Level=base peak intensity.

FIG. 12. HPLC analysis comparing the steroidal products produced by LM9 and LM33 when β-sitosterol is the starting substrate and the culture medium is supplemented with methyl-β-cyclodextrins. The upper line on the graph represents the HPLC trace for the steroidal compounds produced by LM9 and the lower line represents the HPLC trace for the steroidal compounds produced by LM33.

FIG. 13. HPLC analysis comparing the activity of LM9 and LM33 towards 3-oxo-4-cholenic acid as the starting substrate and the culture medium is supplemented with methyl-β-cyclodextrins. The upper line on the graph represents the HPLC trace for the steroidal compounds produced by LM9 (T=72 h) and the lower line represents the HPLC trace for the steroidal compounds produced by LM33 (T=72 h).

FIG. 14. Product ion mass spectrum obtained by LC-MS for LM9 when β-sitosterol is the starting substrate and the culture medium is supplemented with 2-OH-propyl-β-cyclodextrins. Peak at m/z of 373.27 (positive mode) corresponds to production of 3-oxo-4-cholenic acid by LM9. NL: Normalization Level=base peak intensity.

FIG. 15. HPLC analysis of steroidal compounds produced by LM9 β-sitosterol is the starting substrate and the culture medium is supplemented with 2-OH-propyl-β-cyclodextrins. (A) LM9 products at T=24 h; (B) LM9 products at T=48 h; (C) LM9 products at T=72 h; (D) 3-oxo-4-cholenic acid standard (0.025 mg/mL).

FIG. 16. Extracted ion chromatograms obtained by LC-MS for LM9 when 7-oxosterols is the starting substrate and the culture medium is supplemented with 2-OH-propyl-β-cyclodextrins. (A) LM9 products in the presence of 2-OH-propyl-β-cyclodextrins (T=48 h). Peak at 7.74 minutes corresponds to production of 3-oxo-7-hydroxy-4-cholenic acid. (B) LM9 products in the absence of 2-OH-propyl-β-cyclodextrins (T=48 h). Peak at 7.76 minutes corresponds to production of 3-oxo-7-hydroxy-4-cholenic acid. (C) LM9 products in the presence of 2-OH-propyl-β-cyclodextrins but no substrate (T=48 h). NL: Normalization Level=base peak intensity.

FIG. 17. HPLC analysis of steroidal compounds produced by Mycobacterium neoaurum NRRL B-3805 (parent strain) and MneoΔfadE34 when cholesterol is the starting substrate. The upper line on the graph represents the HPLC trace for the steroidal compounds produced by the parent strain (T=72 h) and the lower line represents the HPLC trace for the steroidal compounds produced by MneoΔfadE34.

FIG. 18. HPLC analysis of steroidal compounds produced by Mycobacterium neoaurum NRRL B-3805 (parent strain) and MneoΔfadE34 when β-sitosterol is the starting substrate. The upper line on the graph represents the HPLC trace for the steroidal compounds produced by MneoΔfadE34 (T=72 h) and the lower line represents the HPLC trace for the steroidal compounds produced by the parent strain

FIG. 19. HPLC analysis of steroidal compounds produced by Mycobacterium neoaurum NRRL B-3805 (parent strain) and MneoΔfadE34 when 7-oxo-sterols are the starting substrate. The upper line on the graph represents the HPLC trace for the steroidal compounds produced by MneoΔfadE34 (T=72 h) and the lower line represents the HPLC trace for the steroidal compounds produced by the parent strain.

FIG. 20. HPLC analysis of steroidal compounds produced by MneoΔfadE34 when phytosterol mix (Aturex 90) is the starting substrate and the culture medium is supplemented with methyl-β-cyclodextrins. From bottom to top the traces shown correspond to the steroidal compounds produced by MneoΔfadE34 at T=0 h, 24 h, 48 h, 72 h, 96 h, and 168 h respectively.

FIG. 21. HPLC analysis of steroidal compounds produced by MneoΔfadE34 when 3-oxo-4-cholenic acid is the starting substrate and the culture medium is supplemented with methyl-β-cyclodextrins. From bottom to top the traces shown correspond to the steroidal compounds produced by MneoΔfadE34 at T=0 h, 24 h, 48 h, 72 h, 96 h, and 168 h respectively.

FIG. 22. NMR analysis of steroidal compounds produced by LM33 after fermentation with phytosterol compounds in the presence of hydroxypropyl-β-cyclodextrin. (A) The ¹H-spectrum obtained from the product of the fermentation; (B) Magnified view of the spectrum of FIG. 22A showing peaks in the region 0.65 to 2.55 ppm only; (C) The ¹³C-spectrum obtained from the product of the fermentation; (D) Magnified view of the spectrum of FIG. 22C showing peaks in the region 11 to 58 ppm only. Both the ¹H-spectrum and the ¹³C-spectrum indicate the presence of 3-oxo-4-cholenic acid in the culture; (E) Data parameters used to obtain the ¹H-spectrum shown in FIGS. 22A and 22B; (F) Data parameters used to obtain the ¹³C-spectrum shown in FIGS. 22C and 22D.

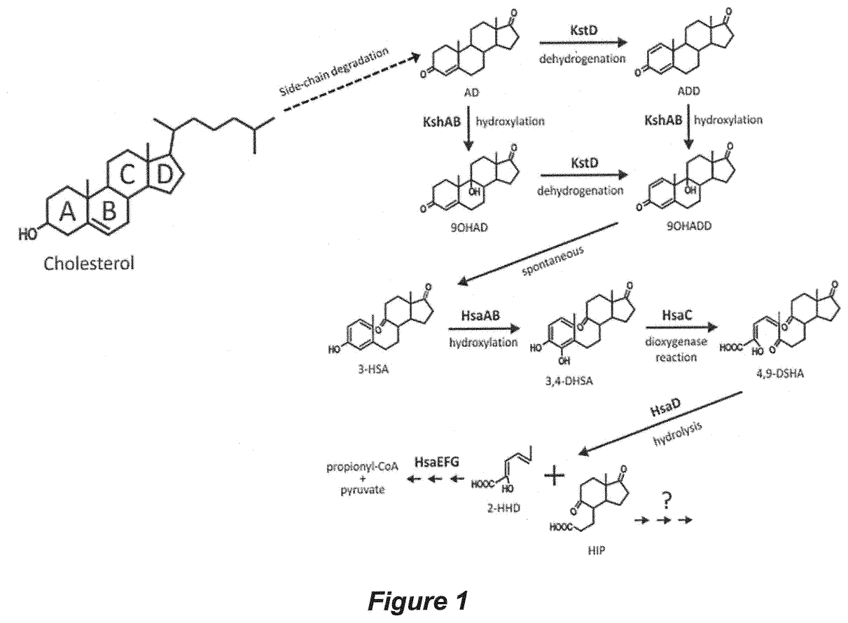

### EXAMPLES

### Example 1—Construction of Strains

### Materials and Methods

### Construction of RG41 Strain

RG41 was originally constructed from the parent strain RG32 which was made by unmarked gene deletion of five homologs of 3-ketosteroid-9α-hydroxylase (kshA1-5) as reported by (Wilbrink et al, 2011. Appl Environ Microbiol., 77(13): 4455-4464).

RG32 was used as parent strain for the construction of R. rhodochrous strain RG41 by deletion of 3 homologs of 3-ketosteroid-Δ1-dehydrogenase (kstDs) as detailed below.

The construction of a mutagenic plasmid for kstD3 unmarked deletion was performed as follows. A genomic library of R. rhodochrous DSM43269 was obtained as explained in (Petrusma et al, 2009. Appl Environ Microbiol., 75(16): 5300-5307), which was used for isolation of a clone (pKSH800; Wilbrink et at., 2011) carrying kshA3 and also kstD3. A 4 kb EcoRI fragment of pKSH800 was ligated into EcoRI-digested pZErO-2.1, which was subsequently digested with Bg/II/EcoRI. Next, a 2.7 kb Bg/II/EcoRI fragment was ligated into BamHI/EcoRI-digested pK18mobsacB, which was then digested with EcoRV/NruI and finally self-ligated, rendering the plasmid pKSH841 for kstD3 gene deletion in R. rhodochrous RG32 strain=>RG32ΔkstD3=strain RG35 (Appendix C).

The construction of a mutagenic plasmid for kstD1 unmarked deletion was performed as follows. Specific kstD1 primers (kstD1-F and kstD1-R, Appendix D) were used for the amplification of a 2.4 kb PCR product that was ligated into EcoRV-digested pBluescript, which was then digested with StuI/StyI, blunt-ended by Klenow and self-ligated. Then, the construct was digested with BamHI/HindIII and, finally, a 1.3 kb BamHI/HindIII fragment was ligated into BamHI/HindIII-digested pK18mobsacB, rendering the plasmid pKSH852 for kstD1 gene deletion in RG35=>RG32ΔkstD1ΔkstD3=strain RG36 (Table Appendix C).

The construction of a mutagenic plasmid for kstD2 unmarked deletion was performed as follows. Chromosomal DNA of R. rhodochrous RG36 was isolated using a genomic DNA isolation kit (Sigma-Aldrich), digested by XhoI, and ligated into XhoI-digested pZErO-2.1. Transformation of E. coli DH5a with the ligation mixture generated a genomic library of approximately 12,000 transformants. A clone carrying the kstD2 gene (pKSD321) was identified by means of PCR using specific kstD2 primers (kstD2-F and kstD2-R, Appendix D) and isolated from the genomic library of strain RG36. Then, pKSD321 was digested with XmnI, self-ligated and subsequently digested with SmaI/XhoI. Finally, a 2.2 kb SmaI/XhoI was ligated into SmaI/SaI-digested pK18mobsacB, rendering the plasmid pKSD326 for the kstD2 gene deletion in RG36=>RG32ΔkstD1ΔkstD2ΔkstD3=strain RG41 (Appendix C).

Mutagenic plasmids were transferred to Escherichia coli S17-1 by transformation and subsequently mobilized to the corresponding R. rhodochrous strain by conjugation as described previously (van der Geize et al, 2001. FEMS Microbiol. Lett., 205(2): 197-202). All mutants were verified by PCR using specific primers (Appendix D) to confirm deletion of the target gene(s).

Therefore, strain RG41 is a kshA null+ΔkstD1ΔkstD2ΔkstD3 mutant (8-fold mutant), which was then used as parent strain for the construction of deletion mutants in genes involved in side-chain degradation of steroids.

### Construction of Deletion Mutation Strains

The single mutant strains LM3 (ΔfadE34#1), LM15 (ΔfadE34#2) were constructed by deletion of fadE34#1 or fadE34#2 from the parent strain RG41 (kshA null+ΔkstD1+ΔkstD2+ΔkstD3).

Unmarked in frame gene deletion mutants were constructed using the sacB counter-selection marker (van der Geize et al, 2001). PCR amplification of the upstream and downstream flanking regions of the target genes was performed from wild-type R. rhodochrous DSM43269 template using the primers listed in Appendix D. The obtained 1.5 kb PCR products (called UP and DOWN) were cloned together into pK18mobsacB vector, yielding pk18_fadE34-UP+DOWN and pk18_fadE34#2-UP+DOWN constructs. pDEL-fadA6, previously constructed by Wilbrink et al., 2011, was used for the deletion of fadA6. Mutagenic plasmids were transferred to Escherichia coli S17-1 by transformation and subsequently mobilized to the corresponding R. rhodochrous strain by conjugation as described previously (van der Geize et al, 2001). All mutants were verified by PCR using specific primers (Appendix 0) to confirm deletion of the target gene(s). LM3 and LM15 single mutant strains were constructed by deletion of fadE34 or fadE34#2, respectively, using RG41 as parent strain.

### Example 2—Bioconversions Using Strains LM3 (ΔfadE34#1) and LM15 (ΔfadE34#2)

### Materials and Methods

Mutant strains were inoculated in 100 ml Luria-Bertani (LB) medium and incubated at 30° C. and 200 rpm for 48 hours. When the OD_{600 nm}=5, the LB preculture was divided into 10 ml cultures and the starting sterol substrate added at 2 mM (dissolved in acetone to 4% final concentration).

The time of addition of the starting sterol substrate was treated as T=0 hours. Cultures were incubated at 30° C. and 200 rpm for several days. 250 μl aliquots were taken from the culture at 0 hours, 24 hours, 48 hours, and 72 hours, and frozen at −20° C. until needed.

Samples were prepared for HPLC and/or LC-MS analysis by thawing at room temperature and adding 1 ml MeOH before briefly vortexing and centrifuging at 4° C. and 12,000 rpm for 10-15 minutes. The supernatants were then filtered (0.2 μm filter size) and analysed by HPLC and/or LC-MS.

HPLC was performed using a Kinetex C18 column (250×4.6 mm, particle size 5 μm). A mobile phase of 80% MeOH and 0.1% formic acid was used at a flow rate of 1 ml/min and a column temperature of 35° C. 20p1 of sample was injected. A 30-minute detection time was used, and steroidal compounds were detected at 254 nm. Quantification of the steroidal products produced was achieved by construction of a calibration line of peak areas measured from a known standard. This was used to calculate the amount of product produced in g/l, followed by back calculation of the percentage yield.

LC-MS analysis was carried out using an Accella1250™ HPLC system coupled with the benchtop ESI-MS Orbitrap Exactive™ (Thermo Fisher Scientific, San Jose, Calif.). A sample of 5 μl was injected into a Reversed Phase C18 column (Shim Pack Shimadzu XR-ODS 3×75 mm) operating at 40° C. and flow rate 0.6 ml/min. Analysis was performed using a gradient from 2% to 95% of acetonitrile:water (adding 0.1% formic acid) as follows: 2 min 2%© acetronitrile, 8 minutes gradient from 2% to 95% acetonitrile, 4 min 95%© acetonitrile. The column fluent was directed to the ESI-MS Orbitrap operating at the scan range (m/z 80-1600 Da) switching positive/negative modes. Voltage parameters for positive mode were: 4.2 kV spray, 57.5 V capillary and 95 V tube lens. Voltage parameters for negative mode were: 3 kV spray, −25V capillary and −75V tube lens. Capillary temperature 325° C., sheath gas flow 70, auxiliary gas off. Thermo XCalibur™ processing software was used for the data analysis. All the products reported in this work were detected in the positive mode (M+H⁺).

### Results

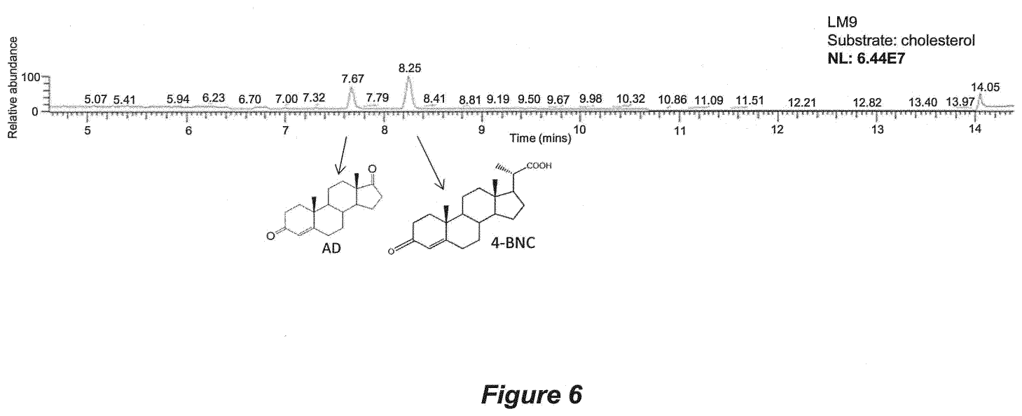

The total ion chromatogram obtained by LC-MS for the LM3 strain shows an accumulation of AD and 4-BNC from the starting cholesterol substrate (FIG. 6), indicating there is no blockage of side-chain degradation in the LM3 single mutant strain. The same result was obtained for the LM15 single mutant strain (data not shown).

### Example 3—Bioconversions using LM9 (ΔfadE34#1/fadE34#2)

### Materials and Methods

The same culture conditions, sample preparation techniques and HPLC/LC-MS protocol were used as outlined in Example 2 above.

### Results

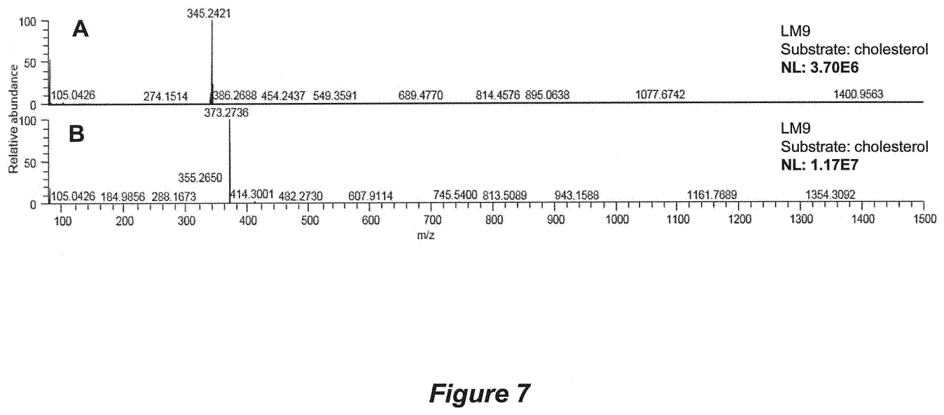

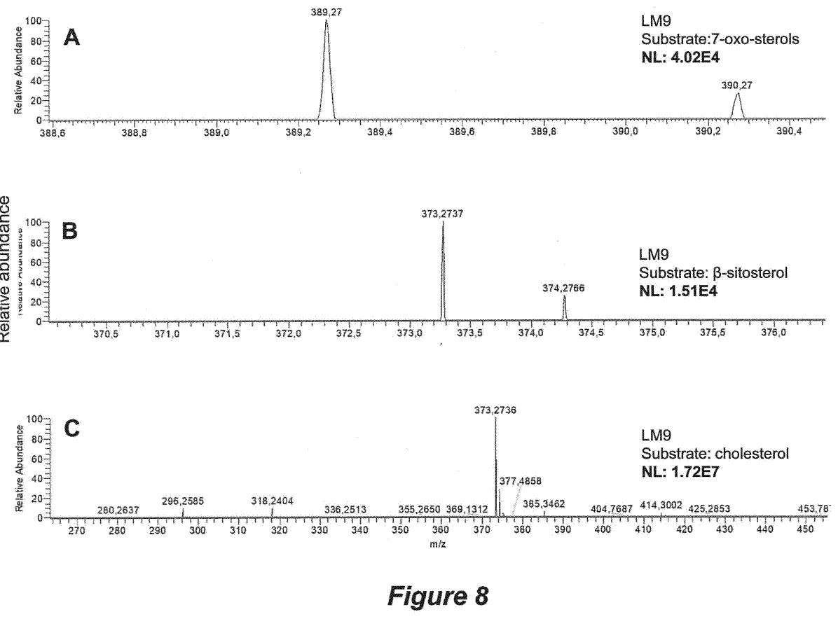

The total ion chromatogram obtained by LC-MS for the LM9 strain (FIG. 7, product ion mass spectra shown) using cholesterol as the starting substrate, shows an accumulation of both 4-BNC (peak at m/z of 345.24, positive mode) (FIG. 7A, top) and 3-oxo-4-cholenic acid (peak at m/z of 373.27, positive mode) (FIG. 7B, bottom). Extracted ion chromatograms, produced by extracting data for the mass to charge ratio (m/z) of the compound of interest, show that 3-oxo-4-cholenic acid is produced by LM9 when cholesterol (FIG. 8C, bottom trace, peak at m/z of 373.27) or β-sitosterol (FIG. 8B, middle trace, peak at m/z of 373.27) is the starting substrate, and that 3-oxo-7-hydroxy-4-cholenic acid is produced when 7-oxo-sterol is the starting substrate (FIG. 8A, top trace, peak at m/z of 389.27). These results indicate that there is some blockage of side-chain degradation in the LM9 strain.

### Example 4—Bioconversions Using Strain LM19 (ΔfadE34#1/ΔfadE34#2 Complemented with kshA5)

### Materials and Methods

### Construction of LM19 Strain

A wild-type copy of the kshA5 gene and its flanking regions was amplified by PCR using the primers kshA5-complem-F and kshA5-complem-R (Appendix D). The obtained PCR product of 2.2 kb was cleaned-up, restricted with BamHI/HindIII and subsequently ligated into pk18mobsacB, yielding the construct pk18+kshA5-complementation. This construct was transformed into E. coli S17-1 and transferred to strain LM9 by conjugation. The resulting complemented mutant LM19, in which the deleted copy of kshA5 was replaced by the wild-type one, was obtained following the same conjugation protocol used for the construction of the mutant strains, as described in van der Geize et al, 2001.

Bioconversions with LM19

As described above, kshA5 and its flanking regions was reintroduced into strain LM9 to produce strain LM19, in which hydroxylase activity is restored to produce variant compounds with a 9-hydroxyl group. The expected compounds accumulated were 3-oxo-9-OH-4-cholenic acid (from β-sitosterol and cholesterol) and 3-oxo-7,9-dihydroxy-4-cholenic acid (from 7-oxo-sterols).

The same culture conditions, sample preparation techniques and HPLC/LC-S protocol were used as outlined in Example 2 above.

### Results

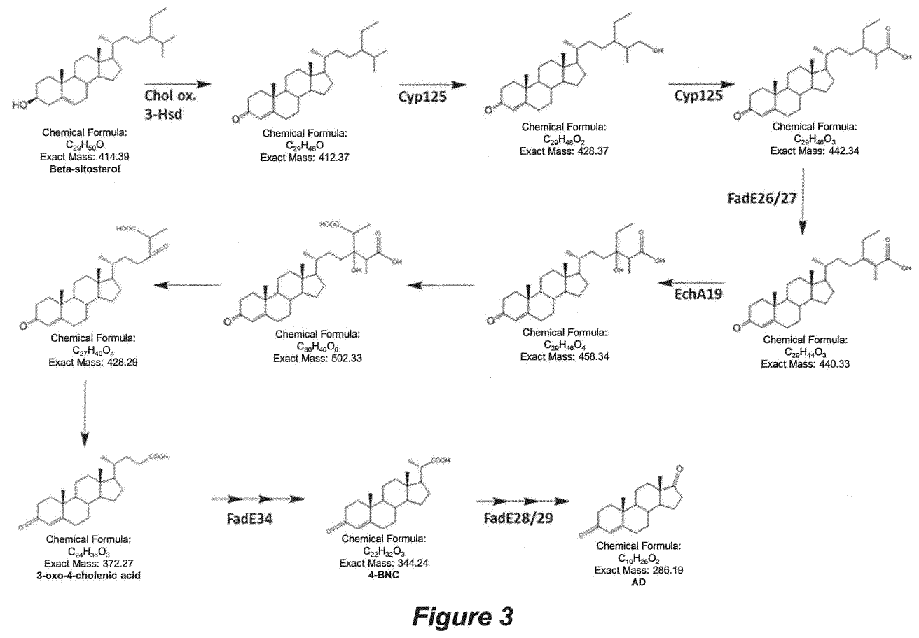

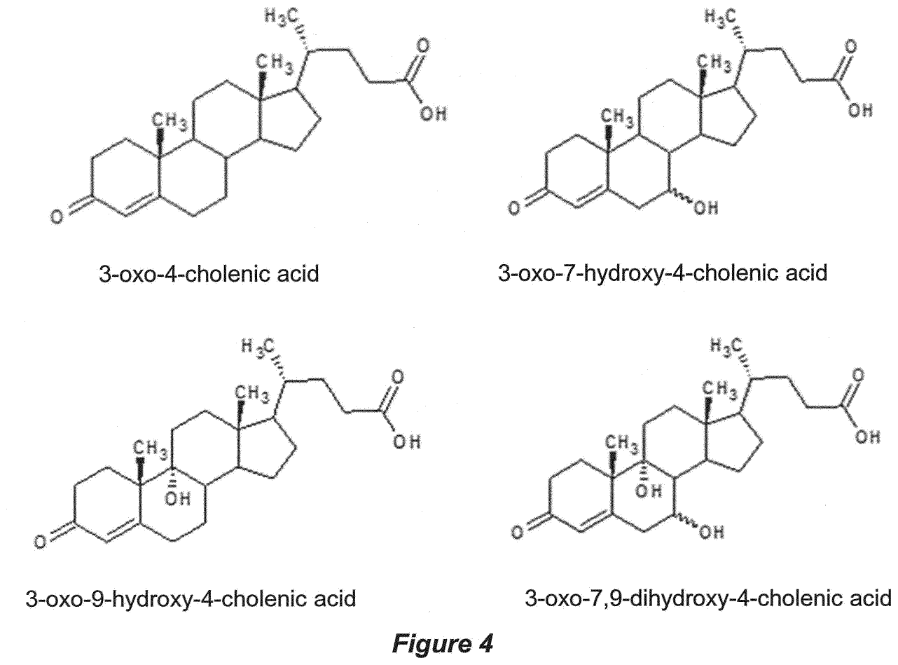

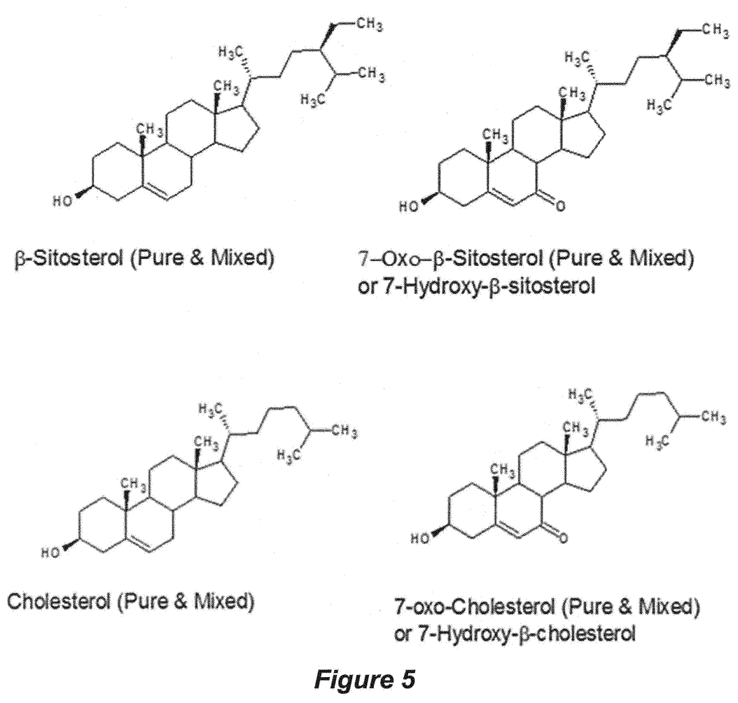

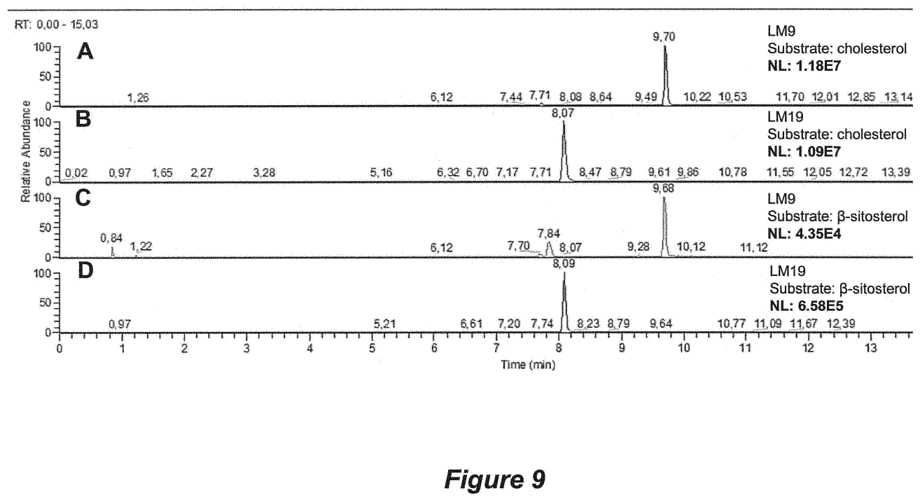

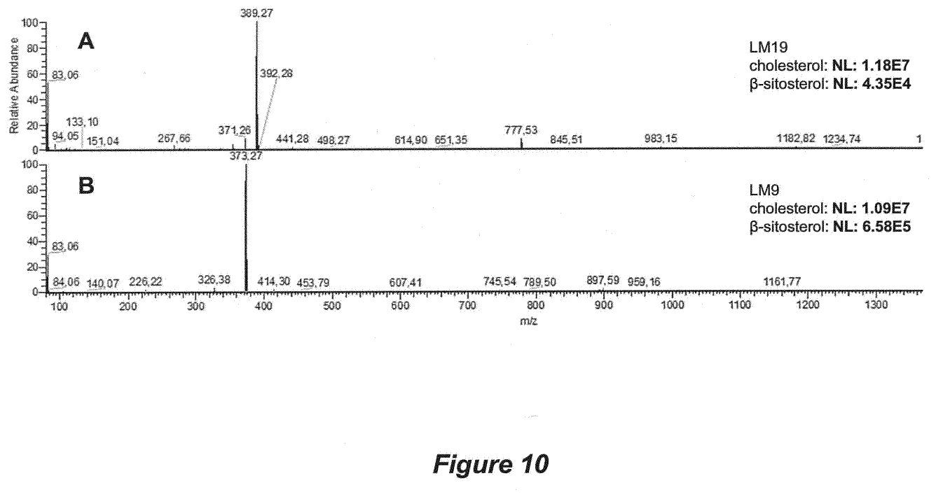

Comparison of the extracted ion chromatograms produced for LM9 and LM19 strains shows that 3-oxo-9-OH-4-cholenic acid (peak at 8.07-8.09 minutes) is produced by LM19 only when the starting sterol is cholesterol or β-sitosterol (FIGS. 9A and 9C respectively) and 3-oxo-4-cholenic acid (peak at 9.68-9.70 minutes) is produced by LM9 only when the starting sterol is cholesterol or β-sitosterol (FIGS. 9B and 9D respectively). Those peaks were confirmed as 3-oxo-9-OH-4-cholenic acid (peak at m/z of approximately 389.27, positive mode) is produced by LM19 when the starting sterol is cholesterol or 1i-sitosterol (FIG. 10A) and 3-oxo-4-cholenic acid (peak at m/z of 373.27, positive mode) is produced by LM9 when the starting sterol is cholesterol or β-sitosterol (FIG. 10B).

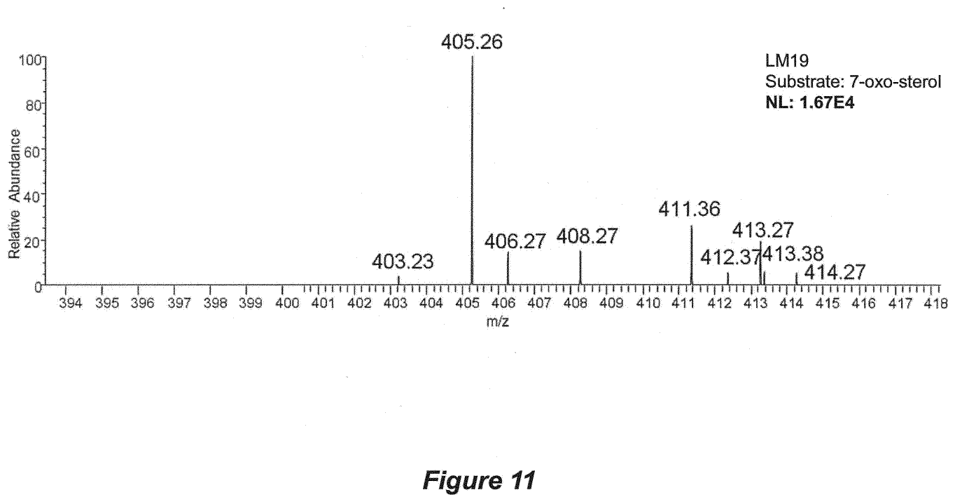

When the starting sterol is 7-oxo-sterol the expected product is 3-oxo-7,9-dihydroxy-4-cholenic acid. The extracted ion chromatogram for LM19 in FIG. 11 has a peak corresponding to 3-oxo-7,9-dihydroxy-4-cholenic acid (peak at m/z of 405.26, positive mode). However, this peak is of lower intensity than those produced for LM19 in FIG. 10. In overview, these results indicate the successful use of LM19 in the production of variant steroidal compounds with a 9-hydroxy group.

### Example 5—Bioconversions Using Strain (ΔfadE34#1/ΔfadE34#2/ΔfadE26)

### Materials and Methods

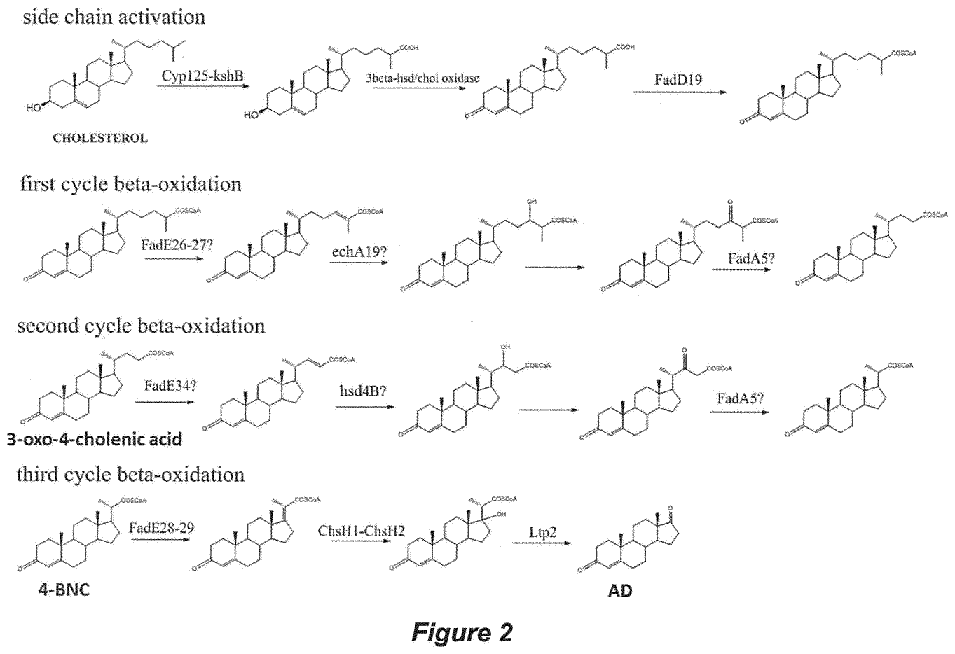

An additional mutant strain ΔfadE34#1/ΔfadE34#2/ΔfadE26 (LM33) was produced by deletion of fadE26 from the LM9 strain. FadE26 is involved in the first cycle of β-oxidation (FIGS. 2 and 3) and may also use 3-oxo-4-cholenic acid as a substrate (Yang et al, 2015. ACS Infect. Dis., 1(2):110-125), thereby limiting its accumulation. Thus, it was thought that deletion of fadE26 might lead to a reduction in unwanted oxidation of 3-oxo-4-cholenic acid.

The same culture conditions, sample preparation techniques and HPLC/LC-MS protocol were used as outlined in Example 2 above.

### Results

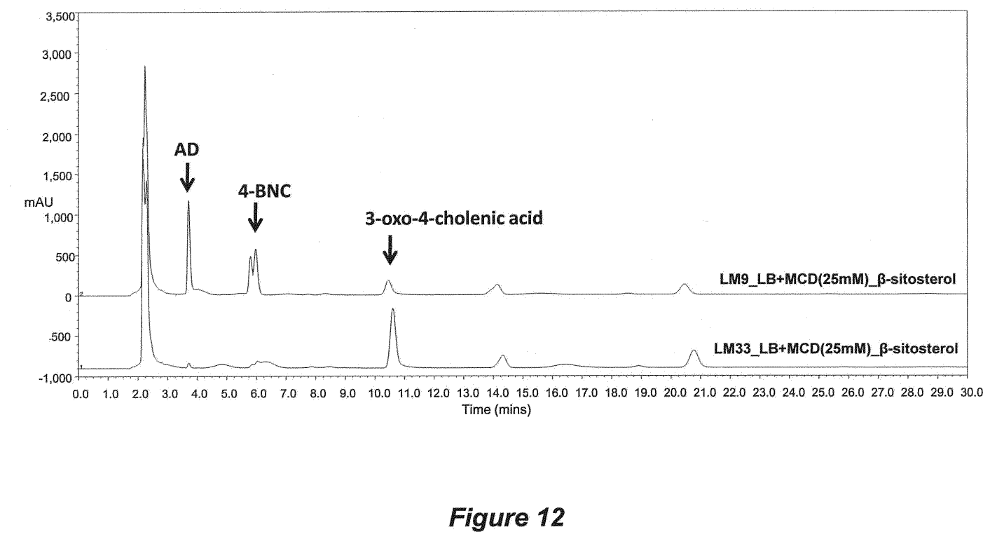

A comparison of the bioconversion of β-sitosterol by the LM9 and LM33 strains in the presence of 25 mM methyl-β-cyclodextrins (MCDs) (see Example 7 below), shows that the major peak in the HPLC trace for the LM33 sample is 3-oxo-4-cholenic acid and the peaks corresponding to AD and 4-BNC are much smaller, while the converse is observed in the HPLC trace for LM9 (FIG. 12). This indicates that the additional deletion of fadE26 in LM33 enables the further accumulation of 3-oxo-4-cholenic acid, suggesting that unwanted oxidation of 3-oxo-4-cholenic acid is reduced.

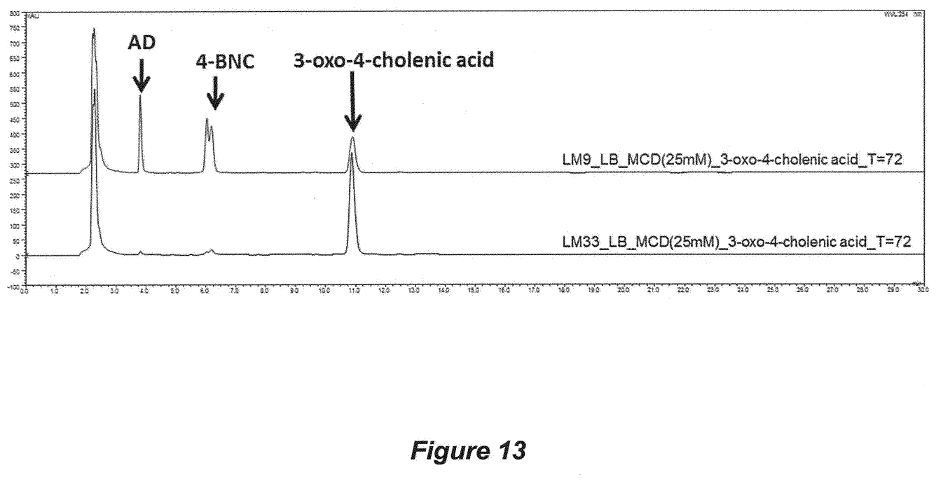

Furthermore, a comparison of the activity of the LM9 and LM33 strains towards 3-oxo-4-cholenic acid as the starting substrate in the presence of 25 mM methyl-β-cyclodextrins (MCDs) shows that the major peak in the HPLC trace for the LM33 sample remains as 3-oxo-4-cholenic acid and peaks corresponding to AD and 4-BNC are very small. In contrast, in the HPLC trace for LM9 (FIG. 13) the peak for 3-oxo-4-cholenic acid is decreased and the peaks for AD and 4-BNC are much more prominent. This indicates that in LM9 the concentration of 3-oxo-4-cholenic acid decreases with time as AD and 4-BNC are formed but in LM33, where fadE26 is also deleted, the conversion of 3-oxo-4-cholenic acid to AD and 4-BNC is significantly reduced. Those results therefore suggest that unwanted oxidation of 3-oxo-4-cholenic acid is reduced in LM33.

### Example 6—Bioconversions Using LM9 in a Culture Medium Supplemented with 2-OH-propyl-β-cyclodextrins

### Materials and Methods

The addition of 2-OH-propyl-β-cyclodextrins to the culture medium was attempted to improve the solubility of the hydrophobic sterol starting compounds.

The LM9 strain was cultured as described in Example 2 until the OD_{600 nm}=5 after approximately 48 hours. The culture was centrifuged at room temperature and 4,500 rpm for 15-20 minutes. The cells were resuspended in the same volume of minimal medium (K₂HPO₄ (4.65 g/l), NaH₂PO₄.H₂O (1.5 g/l), NH₄Cl (3 g/l), MgSO₄.7H₂O (1 g/l), and Vishniac trace element solution (1 ml/l)). This was divided into 10 ml cultures and 25 mM 2-OH-propyl-β-cyclodextrins, 25 mM NaHCO₃ and 2 mM sterols were added in powder form.

The same sample preparation techniques and HPLC/LC-MS protocol were used as outlined in Example 2 above.

### Results

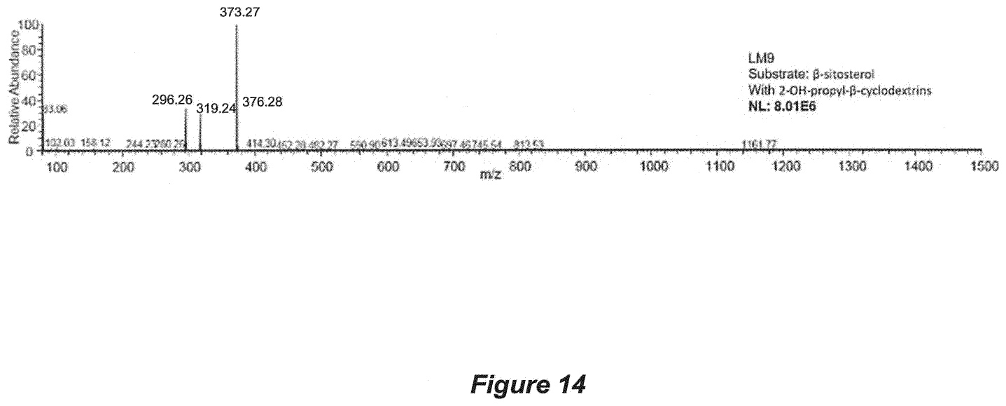

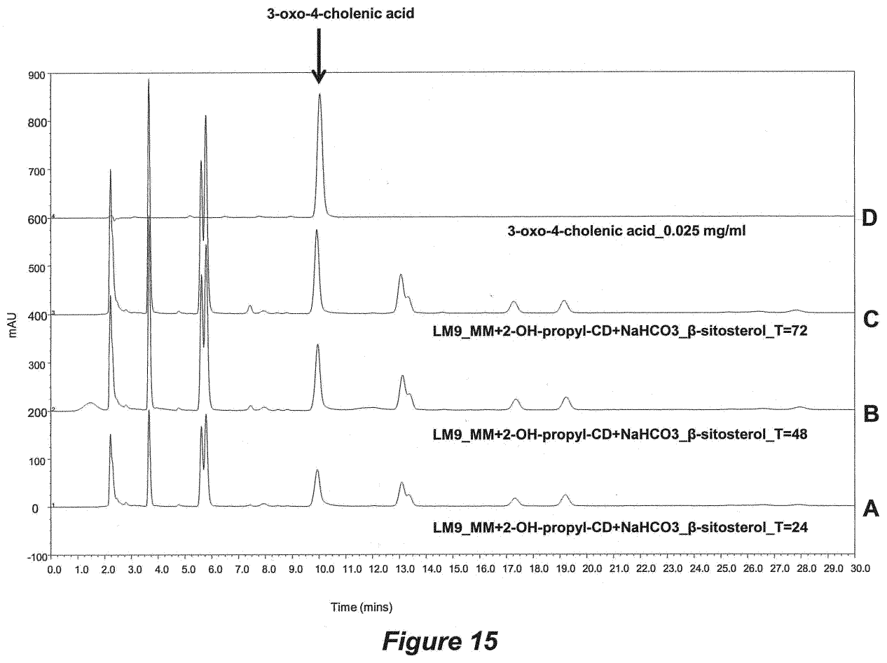

The extracted ion chromatogram obtained by LC-MS of the LM9 strain using β-sitosterol as the starting substrate shows that 3-oxo-4-cholenic acid (peak at m/z of 373.27, positive mode) is produced by LM9 in the presence of 2-OH-propyl-β-cyclodextrins (FIG. 14). In order to quantify the amount of 3-oxo-4-cholenic acid produced HPLC analysis was performed (FIG. 15), with a yield of 11.64% observed in the sample taken at the 72-hour time point (Table 1 below).

TABLE 1
Percentage yields of 3-oxo-4-cholenic acid in LM9 cultures in the
presence of 2-OH-propyl-β-cyclodextrins at T = 24 h/48 h/72 h.

| | Percentage yield (%) of |
|---|---|
| Time point (hours) | 3-oxo-4-cholenic acid |
| 24 | 6.53 |
| 48 | 9.78 |
| 72 | 11.64 |

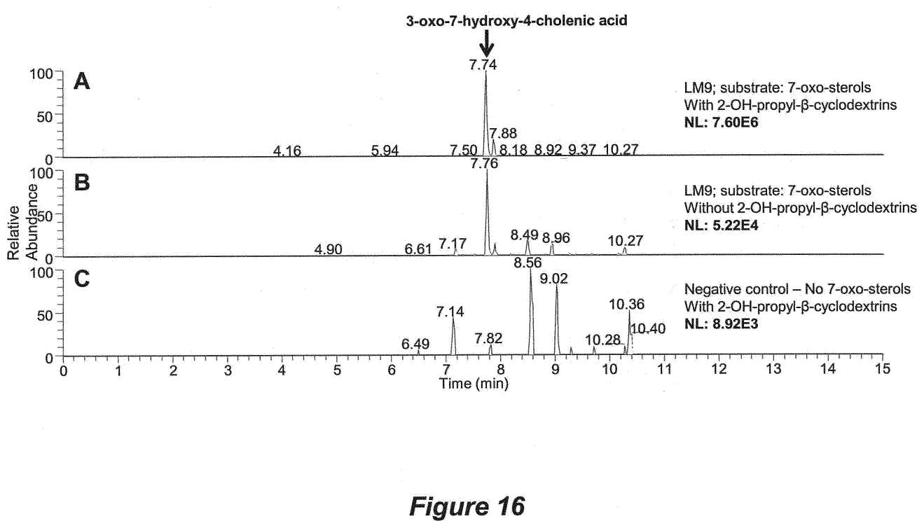

Similar experiments were performed using 7-oxo-sterols as the starting substrate, and the extracted ion chromatograms show the production of 3-oxo-7-hydroxy-4-cholenic acid at T=48 h (FIG. 16). Comparison of the LC-MS spectra in the presence and absence of 2-OH-propyl-β-cyclodextrins (FIGS. 16A and 16B) reveals a more intense base peak (evidenced by the NL values on the traces in FIG. 16) in the presence of 2-OH-propyl-β-cyclodextrins, indicating a higher yield of 3-oxo-7-hydroxy-4-cholenic acid in those cultures. However, due to the lack of an available standard for HPLC quantification, there is no available data on obtainable percentage yields.

Equivalent experiments were carried out in which the culture was not supplemented with NaHCO₃ (data not shown). In those experiments there was no significant difference from the results shown in FIGS. 14, 15, and 16 and presented in Table 1, thereby indicating that the presence of NaHCO₃ is not required to produce a positive effect on yield in cultures supplemented with 2-OH-propyl-β-cyclodextrins.

### Example 7—Bioconversions Using LM9 and LM33 in a Culture Medium Supplemented with Methyl-β-Cyclodextrins

### Materials and Methods

The addition of methyl-β-cyclodextrins to the culture medium was attempted to further improve the solubility of the hydrophobic sterol starting compounds.

The LM9 strain was cultured as described in Example 2 until the OD_{600 nm}=5 after approximately 48 hours. The culture was centrifuged, and the cells resuspended in the same volume of minimal medium, as described in Example 6. This was divided into 10 ml cultures and 25 mM methyl-β-cyclodextrins and 2 mM sterols were added in powder form.

In an attempt to further maximise the yield of 3-oxo-4-cholenic acid, methyl-β-cyclodextrins were added to the LM33 strain (see FIG. 12). The LM33 strain was cultured in LB medium as described in Example 2 until the OD_{600 nm}=5 after approximately 48 hours. Then, the preculture was divided into 10 ml cultures and 25 mM methyl-β-cyclodextrins and 2 mM sterols were added in powder form. Alternatively, the culture was centrifuged, and the cells resuspended in the same volume of minimal medium. This was divided into 10 ml cultures and 25 mM methyl-β-cyclodextrins and 2 mM sterols were added in powder form.

The same sample preparation techniques and HPLC/LC-MS protocol were used as outlined in Example 2 above.

### Results

Table 2 below summarises the maximum percentage yields of 3-oxo-4-cholenic acid obtained by HPLC analysis for LM9 in the presence of methyl-β-cyclodextrins using β-sitosterol as the starting substrate and compares those yields to the yields obtained in the presence of 2-OH-propyl-β-cyclodextrins (see Example 6 above). The overall result indicates that yields are higher in the presence of methyl-β-cyclodextrins.

TABLE 2
Percentage yields of 3-oxo-4-cholenic acid in LM9 cultures
supplemented with cyclodextrins at T = 72 h.

| | Percentage yield of |
|---|---|
| Culture conditions | 3-oxo-4-cholenic acid (%) |
| 2-OH-propyl-cyclodextrin (25 mM), 72 h | 11.64 |
| Methyl-β-cyclodextrins (25 mM), 72 h | 23.08 |

Quantification of the amount of product produced by LM9 in the presence of methyl-β-cyclodextrins (25 mM) was carried out using HPLC analysis. β-sitosterol was the starting substrate and the analysed sample was collected at the 72-hour timepoint. The percentage yields were calculated as outlined in Example 2 above and are presented in Table 3 below.

TABLE 3
Percentage yields of steroidal compounds in LM9 cultures
supplemented with methyl-β-cyclodextrins (25 mM) at T = 72h.

| | Percentage yield (%) of |
|---|---|
| Steroidal compound | steroidal compound |
| 3-oxo-4-cholenic acid | 23.08 |
| 4-BNC | 14.80 |
| AD | 19.00 |

Similarly, Table 4 below compares bioconversions in LM9 in the presence of methyl-β-cyclodextrins using 7-oxo-sterols as the starting substrate. Due to the lack of available standard for 3-oxo-7-hydroxy-4-cholenic acid, peak areas obtained by HPLC are compared rather than expressed as a percentage yield. However, the results still demonstrate that larger peak areas are achieved in the presence of methyl-β-cyclodextrins compared with 2-OH-propyl-β-cyclodextrins.

TABLE 4
Peak area measurements for 3-oxo-7-hydroxy-4-cholenic acid in
LM9 cultures supplemented with cyclodextrins (25 mM) at T = 72h.

| Culture conditions | Peak area |
|---|---|
| 2-OH-propyl-cyclodextrin (25 mM), 72 h | 21.21 |
| Methyl-β-cyclodextrins (25 mM), 72 h | 44.22 |

Table 5 below summarises the percentage yields of 3-oxo-4-cholenic acid obtained by HPLC analysis for LM33 using both cholesterol and β-sitosterol as starting substrates and culturing in both LB and minimal medium in the presence of methyl-β-cyclodextrins. Comparing the data in Table 3 above and Table 5 below shows that culturing LM33 in the presence of methyl-β-cyclodextrins results in the highest percentage yield of 3-oxo-4-cholenic acid when β-sitosterol is the starting substrate.

TABLE 5
Percentage yields of 3-oxo-4-cholenic acid in LM33 cultures
supplemented with methyl-β-cyclodextrins at T = 72 h.

| | Percentage yield of |
|---|---|
| Culture conditions | 3-oxo-4-cholenic acid (%) |
| B-sitosterol LB medium, 72 h | 37.31 |
| B-sitosterot, minimal medium, 72 h | 39.74 |
| Cholesterol B medium, 72 h | 50.51 |
| Cholesterol, minimal medium, 72 h | 66.82 |

### Example 8—Bioconversions Using 3 in Culture Medium Supplemented with Organic Solvents and Cyclodextrins

### Materials and Methods

The LM33 strain was cultured as described in Example 1 until the OD_{600 nm}=5 after approximately 48 hours. The culture was centrifuged at 4,500 rpm at room temperature for 15-20 mins. The cells were resuspended in the same volume of minimal medium and the culture divided into 10 ml cultures. 2 mM β-sitosterol was added dissolved in ethanol (5% or 10% final volume/volume concentration) and different amounts of methyl-β-cyclodextrins (5 mM, 12.5 mM, or 25 mM) were added in powder form.

The same sample preparation techniques and HPLC/LC-MS protocol were used as outlined in Example 2 above.

### Results

HPLC data for all concentrations of ethanol and methyl-β-cyclodextrins was processed as described in Example 2 to obtain the percentage yields of 3-oxo-4-cholenic acid displayed in Table 6 below. Overall, the use of 5% ethanol and 5 mM methyl-β-cyclodextrins in combination results in the highest percentage yield.

TABLE 6
Percentage yields of 3-oxo-4-cholenic acid in LM33 cultures
supplemented with methyl-β-cyclodextrins
and ethanol at T = 72 h.

| | Percentage yield of |
|---|---|
| Sample conditions | 3-oxo-4-cholenic acid (%) |
| 0 mM MCDs, 5% ethanol, 72 h | 6.97 |
| 5 mM MCDs, 5% ethanol, 72 h | 71.30 |
| 12.5 mM MCDs, 5% ethanol, 72 h | 65.11 |
| 25 mM MCDs, 5% ethanol 72 h | 62.16 |
| 5 mM MCDs, 10% ethanol, 72 h | 13.05 |
| 12.5 mM MCDs, 10% ethanol 72 h | 34.01 |
| 25 mM MCDs, 10% ethanol 72 h | 32.24 |

### Example 9—Bioconversions Using Mycobacterium neoaurum NRRL B-3805 ΔfadE34 (MneoΔfadE34)

### Materials and Methods

The Mycobacterium neoaurum NRRL B-3805 ΔfadE34 strain was produced by introducing a deletion of fadE34 into the parent strain NRRL B-3805 (Marsheck et al, 1972. Applied Microbiology, 3(1):72-77), with the aim of preventing the oxidation of 3-oxo-4-cholenic acid. This followed the same strategy described in Example 1, using the parent strain NRRL B-3805 template and the primers listed in Appendix D, pk18_fadE34 Mneo-UP+DOWN plasmid was constructed. This mutagenic plasmid was transferred to NRRL B-3805 strain by electroporation (2.5 kV, 25 μF, 600Ω). The mutant strain was verified by PCR using specific primers (Appendix D) to confirm deletion of fadE34.

MneoΔfadE34 precultures were grown to an OD_{600 nm}=5 (˜72 h at 37° C.). The culture was centrifuged, and the cells suspended in the same volume of minimal medium. 2 mM of the starting steroid substrate was added in powder form.

The same sample preparation techniques and HPLC/LC-MS protocol were used as outlined in Example 2 above.

### Results

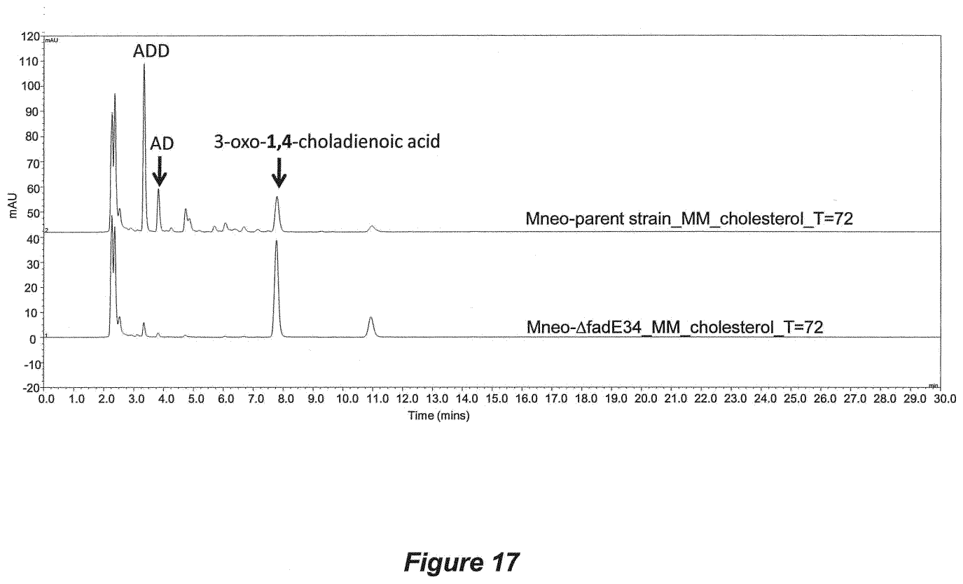

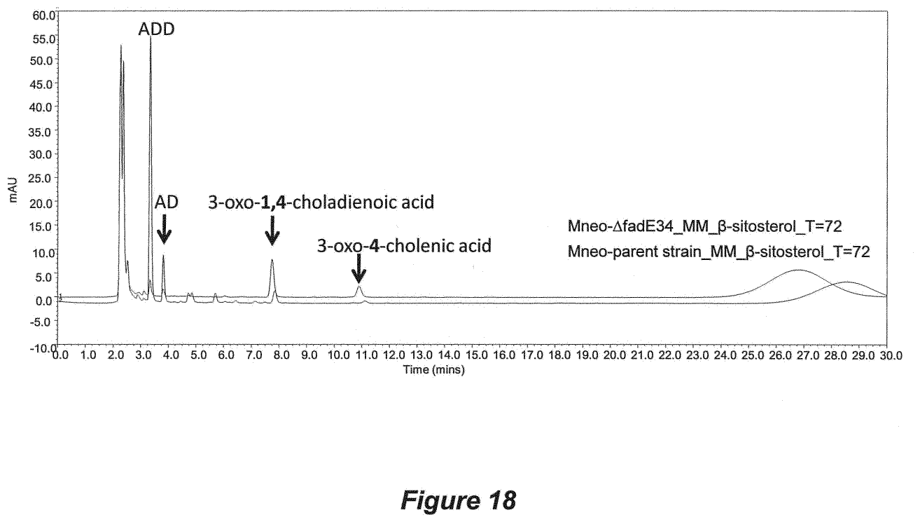

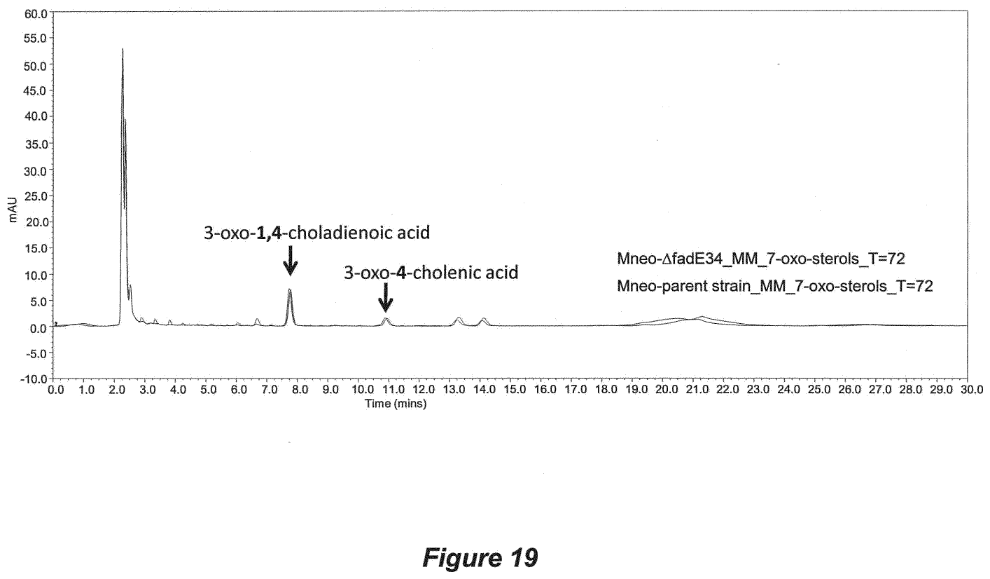

The HPLC traces of FIG. 17, FIG. 18 and FIG. 19 compare the compounds produced by the Mneo-parent strain and MneoΔfadE34 strain when cholesterol, β-sitosterol and 7-oxosterols are the respective starting substrates. In the case of cholesterol (FIG. 17) and β-sitosterol (FIG. 18), the MneoΔfadE34 strain accumulates higher levels of 3-oxo-4-cholenic acid and lower levels of AD and ADD than the Mneo-parent strain. These results indicate that the MneoΔfadE34 strain is blocked in side-chain oxidation at the 3-oxo-4-cholenic acid step. The Mneo parent strain NRRL B-3805 was described as lacking KSH and KstD, however, it was observed that there is also a peak that corresponds to production of 3-oxo-1,4-choladienoic acid, indicating that MneoΔfadE34 (and therefore the parent strain NRRL B-3805) may have residual KstD activity.

When 7-oxosterols are the starting substrate (FIG. 19), the traces obtained for the Mneo parent strain NRRL B-3805 and MneoΔfadE34 are very similar, indicating that 7-OH compounds are not able to be accumulated.

### Example 10—Bioconversions Using Mycobacterium neoaurum NRRL B-3805 ΔfadE34 (MneoΔfadE34) in Culture Medium Supplemented with Methyl-β-Cyclodextrins

### Materials and Methods

The same strains and culture conditions were used as outlined in Example 9 above, and 25 mM methyl-β-cyclodextrins was added in powder form. 2 mM phytosterol mix (Aturex 90) or 3-oxo-4-cholenic acid were added as the starting compounds. The same sample preparation techniques and HPLC/LC-MS protocol were used as outlined in Example 2 above.

### Results

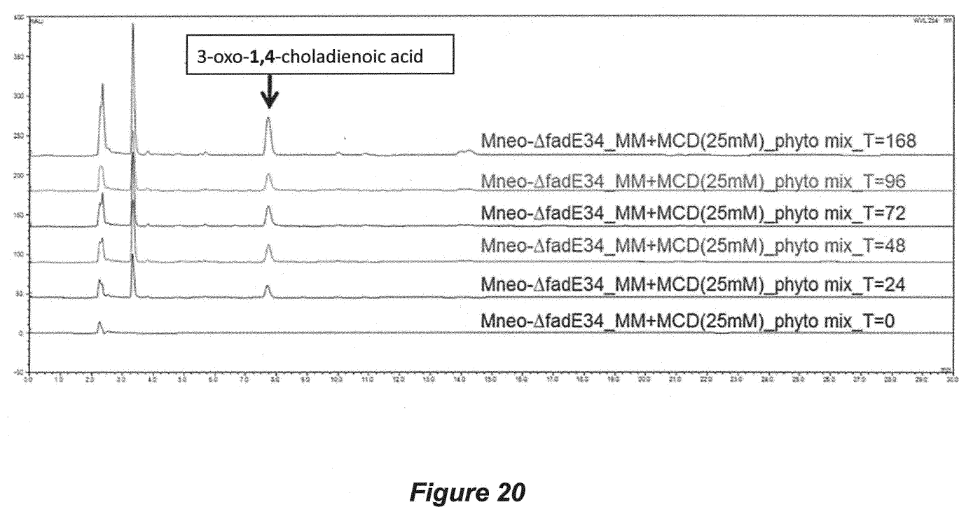

Mneo-ΔfadE34 accumulates a possible peak of 3-oxo-1,4-choladienoic acid when those cells are cultured in minimal medium in the presence of methyl-β-cyclodextrins and phytosterol mix is the starting substrate (FIG. 20).

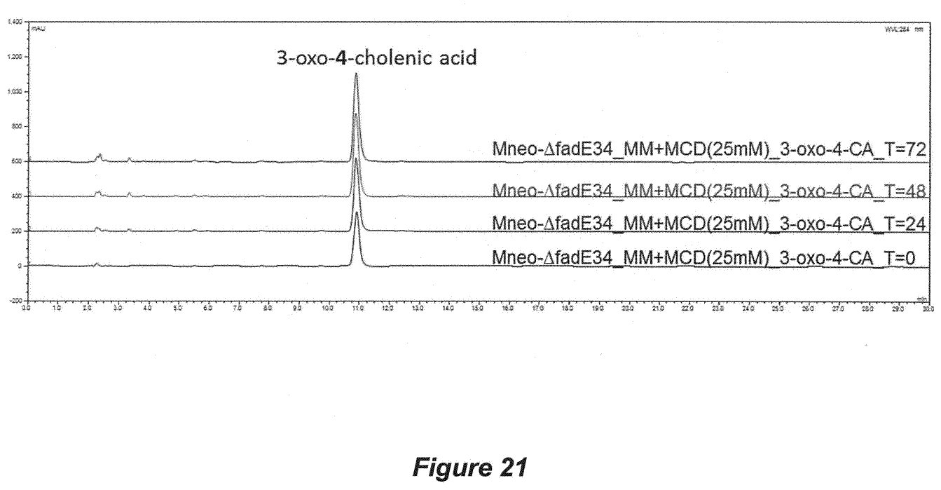

When 3-oxo-4-cholenic acid is the starting substrate, there is no accumulation of 3-oxo-1,4-cholenic acid (FIG. 21), indicating it is likely that its production is not activated by the presence of 3-oxo-4-cholenic acid.

### Example 11—Bioconversions Using in Culture Medium Supplemented with Hydroxy-Propyl-β-Cyclodextrin

### Materials and Methods

The bioconversion was carried out with growing cells i.e. with bioconversion reagents added to the reactor at the beginning of the fermentation. A pre-culture was prepared as follows:
1) 50 mL LB medium was added to 100 mL conical flask;
2) 200 μL R. rhodochrous LM33 was inoculated from glycerol stock;
3) The culture was incubated at 400 RPM on an orbital shaker for 48 hours at 30° C.;
4) 1% (5 mL) of OD 5 culture was inoculated into the bioreactor.

The bioreactor was loaded with (final concentrations in brackets): Tryptone (10 g/L); Yeast Extract (10 g/L); NaCL (0.5 g/L); Antifoam DR204 (0.015 g/L); Hydroxy-propyl-β-Cyclodextrin (23.3 g/L); a premade mixture of Phytosterols AS-7 (70 g/L) and Tween-80 (17.5 g/L); and water. The mixture was autoclaved in the reactor at 121° C. for 3 minutes. The bioconversion was run at 30° C., pH 7.0 with aeration from surface at 200 mL/min and dO₂ set point at 40%.

The initial growth lasted for less than 12 hours as judged from oxygen consumption and a slight CO₂ production. After 48 hours from the start of the experiment there was no CO₂ production and the bioconversion was reinoculated from a fresh pre-culture, at an inoculation rate of 10% (50 mL). After the second inoculation, there was also an initial oxygen consumption phase, which lasted for 6 hours, again followed by a reduction in oxygen consumption. However, after that reduction the culture recovered and started consuming oxygen and producing CO₂ again.

Formation of 3-oxo-4-cholenic acid was detected at the 112th hour. The experiment was concluded after 160 hours, at which point the product concentration had reached 6.09 mM.

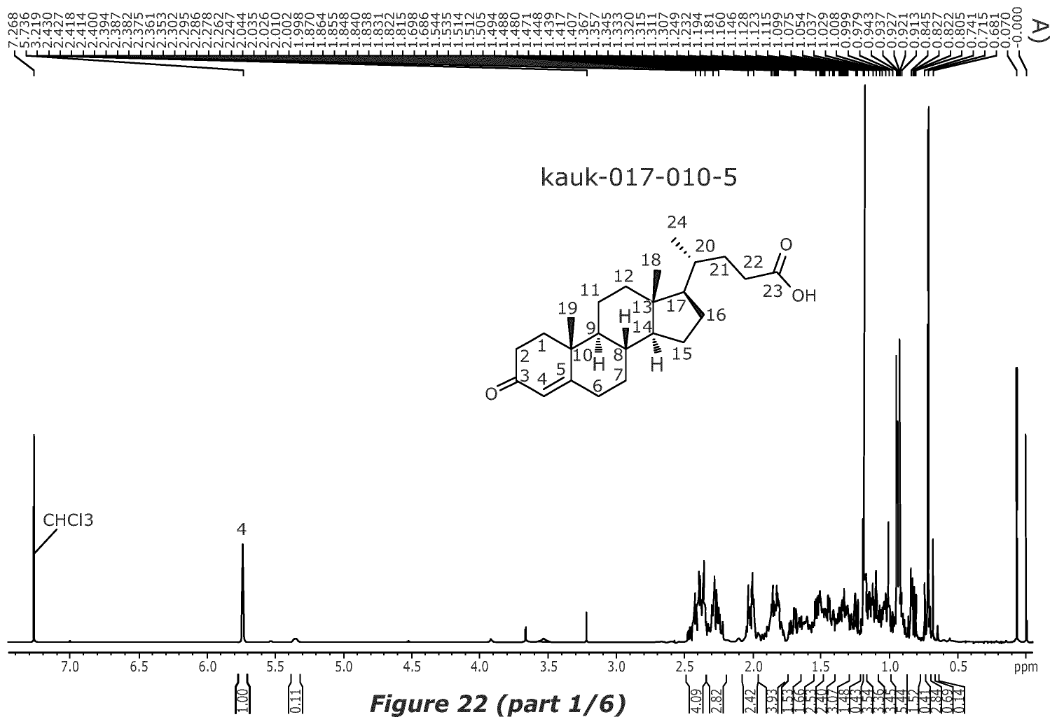

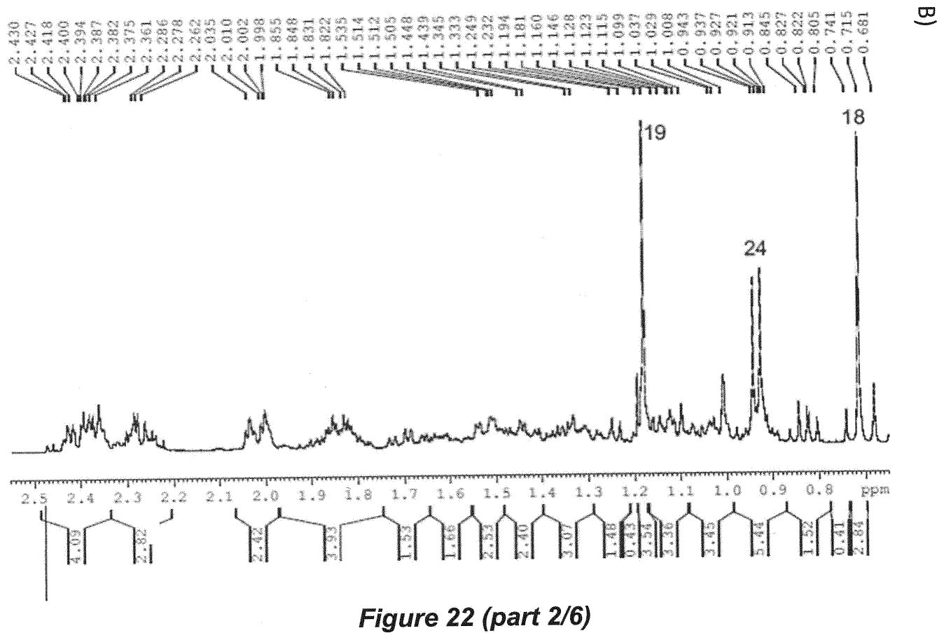

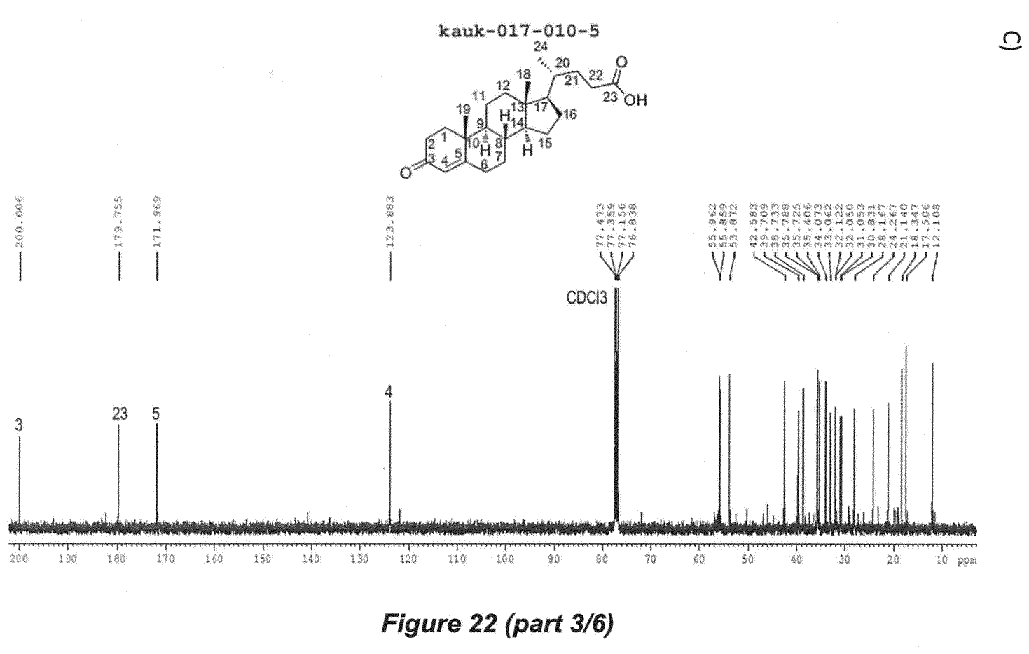

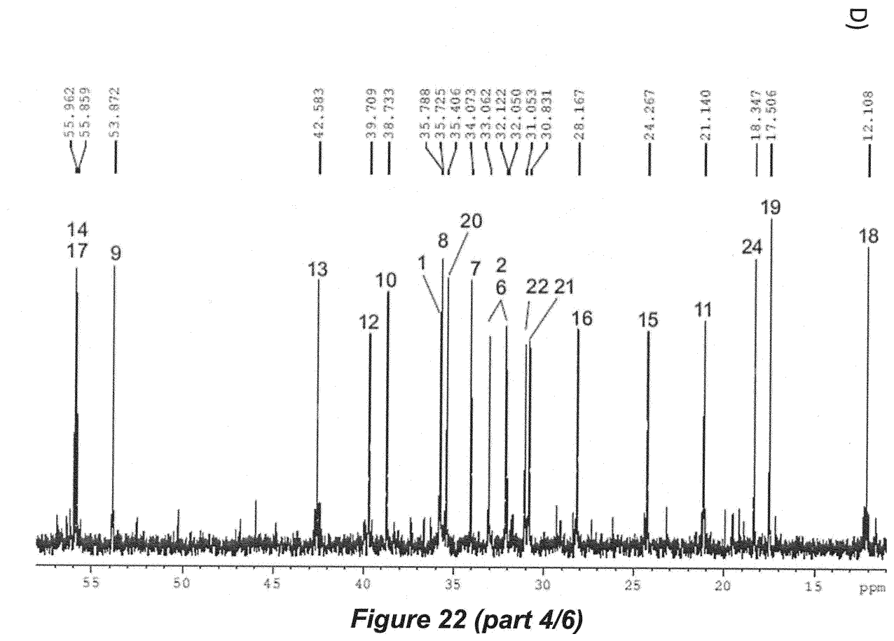

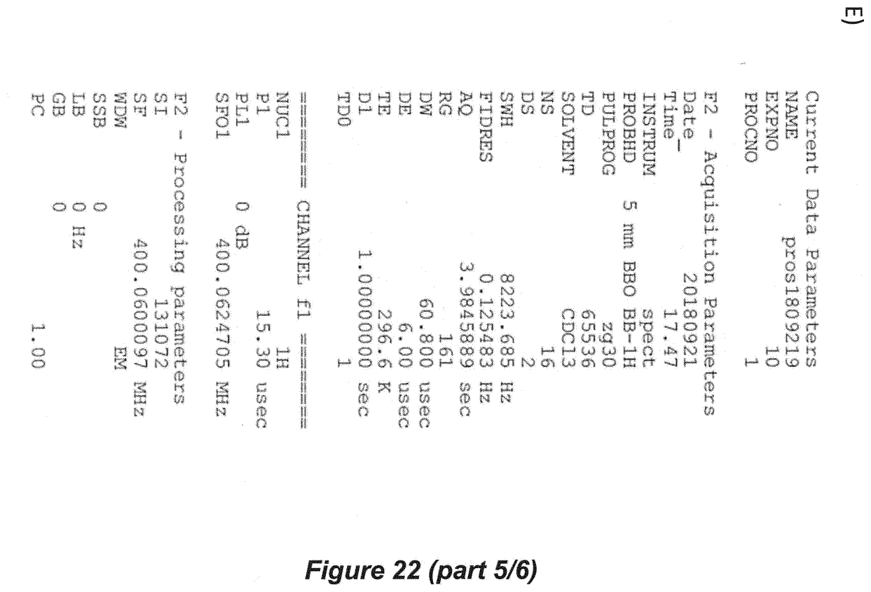

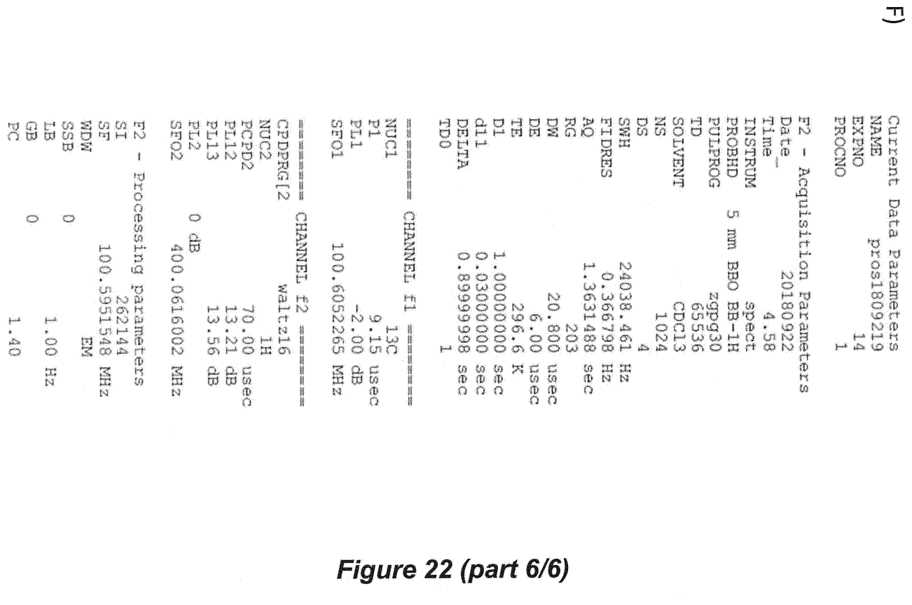

The biomass and unreacted phytosterols were separated by first increasing the pH of the culture solution to pH 10 by the addition of 2M NaOH, followed by centrifugation at 4700 g for 10 minutes at 4° C., affording a clear solution (453 g) containing the product. From this solution, 360 g (pH=7.2) was extracted with 4×100 ml MTBE, then adjusted to pH=2.1 with diluted HCl and extracted again with 2×100 ml toluene. The majority of 3-oxo-4-cholenic acid was detected in MTBE extracts and a minority in toluene extracts. The extracts were evaporated to dryness and pooled by dissolving in MTBE. The solution was washed with diluted HCl and concentrated on rotavap. From the obtained residue, 3-oxo-4-cholenic acid was precipitated by overnight stirring. The identity of 3-oxo-4-cholenic acid was confirmed by NMR (FIG. 22).

### Results

The spectra of FIG. 22 confirm the identity of the isolated product as 3-oxo-4-cholenic acid. FIGS. 22(A) and (B) depict the ¹H-spectrum and FIGS. 22(C) and (D) depict the ¹³C-spectrum obtained from the products. The labelling of the peaks corresponds to the functional groups depicted in the formula of 3-oxo-4-cholenic acid shown in FIGS. 22(A) and (C).

APPENDIX A
Nucleotide sequences

| | GENBANK | |
|---|---|---|
| Name and SEQ ID NO. | Accession No. | Nucleotide sequence |
| kshA1 Rhodococcus | HQ425873.1 | GTGAGCCTCGGCACTTCCGAACAATCCGAAATCCGTGA |
| rhodochrous (SEQ ID NO: 1) | | GATCGTCGCCGGGTCGGCTCCCGCCCGCTTCGCCCGCG |
| | | GCTGGCACTGCCTCGGCCTGGCGAAGGATTTCAAGGAC |
| | | GGCAAGCCGCATTCCGTGCACGCCTTCGGTACCAAACT |
| | | CGTGGTGTGGGCCGACAGCAACGACGAGATCAGGATCC |
| | | TCGACGCGTACTGCCGGCACATGGGCGGCGATCTCAGC |
| | | CAGGGCACCGTCAAGGGCGACGAGATCGCGTGCCCGTT |
| | | CCACGACTGGCGCTGGGGCGGCAACGGCCGCTGCAAGA |
| | | ACATCCCGTACGCACGTCGTGTTCCCCCGATCGCGAAG |
| | | ACCCGCGCGTGGCACACGCTCGATCAGGACGGGCTGCT |
| | | GTTCGTCTGGCACGACCCCCAGGGCAATCCGCCGCCGG |
| | | CCGACGTGACGATCCCGCGCATCGCGGGTGCGACGAGC |
| | | GACGAGTGGACCGACTGGGTCTGGTACACCACCGAGGT |
| | | CGACACCAACTGCCGCGAGATCATCGACAACATCGTCG |
| | | ACATGGCGCACTTCTTCTACGTGCACTACTCCTTCCCG |
| | | GTGTACTTCAAGAACGTCTTCGAAGGACACGTCGCCAG |
| | | CCAGTTCATGCGCGGTCAGGCCCGTGAGGACACCCGTC |
| | | CGCACGCGAACGGTCAACCGAAGATGATCGGAAGCCGA |
| | | TCCGATGCAAGCTATTTCGGCCCGTCCTTCATGATCGA |
| | | CGATCTCGTCTACGAGTACGAGGGATACGACGTCGAGT |
| | | CGGTCCTCATCAACTGCCACTACCCGGTCTCCCAGGAC |
| | | AAGTTCGTCCTGATGTACGGCATGATCGTCAAGAAGTC |
| | | CGACCGTCTCGAGGGCGAGAAGGCGTTGCAGACCGCGC |
| | | AGCAGTTCGGCAACTTCATCGCGAAGGGTTTCGAGCAG |
| | | GACATCGAGATCTGGCGCAACAAGACCCGCATCGACAA |
| | | CCCGCTCCTGTGCGAGGAGGACGGCCCCGTCTACCAGC |
| | | TGCGTCGCTGGTACGAGCAGTTCTACGTCGACGTCGAG |
| | | GACGTCGCGCCCGAGATGACCGACCGCTTCGAGTTCGA |
| | | GATGGACACCACCCGTCCCGTCGCGGCGTGGATGAAGG |
| | | AGGTCGAGGCGAACATCGCCCGCAAGGCCGCCCTCGAC |
| | | ACGGAAACTCGTTCTGCACCAGAGCAGTCCACCACCGC |
| | | GGGCTAG |
| kshA2 Rhodococcus | HQ425874.1 | GTGGGTTCCACAGACACCGAAGATCAGGTCCGCACCAT |
| rhodochrous (SEQ ID NO: 2) | | CGATGTGGGCACGCCGCCGGAGCGCTACGCGCGAGGAT |
| | | GGCACTGCCTGGGGCTCGTACGCGATTTCGCCGACGGC |
| | | AAGCCCCACCAGGTCGACGCGTTCGGGACCTCGCTCGT |
| | | GGTGTTCGCCGGTGAGGACGGAAAGCTCAACGTTCTGG |
| | | ACGCCTACTGCAGGCACATGGGTGGAAATCTGGCCCAG |
| | | GGATCCGTGAAGGGCAACACCATCGCCTGTCCGTTCCA |
| | | CGACTGGCGCTGGCGCGGTGACGGGAAGTGTGCCGAGA |
| | | TTCCCTATGCGCGCCGTGTTCCACCGCTCGCCCGTACC |
| | | CGGACGTGGCCGGTGGCGGAGGTGAGCGGTCAGCTCTT |
| | | CGTGTGGCACGACCCGCAGGGCAGCAAGCCGCCGGCGG |
| | | AGCTCGCCGTTCCGGAGGTTCCCACCTACGGCGATCCC |
| | | GGGTGGACCGACTGGGTGTGGAACTCGATCGAGGTGAC |
| | | CGGATCCCACTGTCGCGAGATCGTGGACAACGTCGTCG |
| | | ACATGGCGCACTTTTTCTACGTCCACTACGGGATGCCG |
| | | ACCTACTTCCGAAACGTGTTCGAAGGTCATACGGCCAC |
| | | CCAGGTCATGCGGTCCCTGCCCCGGGCGGACGCCGTAG |
| | | GCGTCAGCCAGGCCACCAATTACAGTGCCGAGAGCAGA |
| | | TCCGATGCAACGTATTACGGTCCCTCGTACATGATCGA |
| | | CAAGCTGTGGAGCGCCGGCCGTGATCCCGAGTCGACGC |
| | | CGAACATCTATCTGATCAACTGCCACTACCCCATCTCT |
| | | CCGACCTCCTTCCGCCTGCAGTACGGCGTGATGGTGGA |
| | | AAGGCCCGAGGGAGTGCCCCCGGAGCAGGCGGAACAGA |
| | | TCGCCCAGGCCGTCGCCCAGGGCGTCGCGATCGGATTC |
| | | GAGCAGGACGTCGAGATCTGGAAGAACAAGTCGCGGAT |
| | | CGACAACCCCCTGCTGTGCGAGGAGGACGGTCCCGTCT |
| | | ACCAACTGCGGCGGTGGTACGAACAGTTCTACGTCGAC |
| | | GTCGAAGACATCCGACCCGAGATGGTCAACCGGTTCGA |
| | | GTACGAGATCGACACCACGCGCGCCCTGACGAGCTGGC |
| | | AGGCCGAAGTCGACGAGAACGTCGCGGCCGGACGTAGT |
| | | GCCTTCGCCCCGAACCTCACCCGGGCTCGTGAAGCAGC |
| | | CTCCGCCGAATCGGGATCCTGA |
| kshA3 Rhodococcus | HQ425875.1 | ATGGCACAGATTCGCGAGATCGACGTCGGAGAGGTCCG |
| rhodochrous (SEQ ID NO: 3) | | GACGCGTTTCGCGCGAGGCTGGCACTGCCTCGGCCTCA |
| | | GTCGCACGTTCAAGGACGGCAAGCCCCACGCCGTCGAG |
| | | GCCTTCGGCACGAAACTCGTGGTGTGGGCCGACAGCAA |
| | | CGGCGAACCGAAGGTGCTCGACGCGTACTGCCGTCACA |
| | | TGGGCGGCGACCTGTCACAGGGCGAGATCAAGGGCGAT |
| | | TCGGTTGCGTGCCCGTTCCACGACTGGCGCTGGGGCGG |
| | | CAACGGCAAGTGCACGGACATCCCGTATGCCAGGCGCG |
| | | TTCCCCCGCTGGCCCGCACCCGTTCGTGGATAACGATG |
| | | GAGAAGCACGGCCAGCTGTTCGTGTGGAACGACCCCGA |
| | | GGGCAACACCCCGCCCCCGGAGGTCACGATCCCCGAGA |
| | | TCGAGCAGTACGGCTCGGACGAGTGGACGGACTGGACC |
| | | TGGAACCAGATCCGGATCGAAGGTTCCAACTGTCGCGA |
| | | GATCATCGACAACGTCGTCGACATGGCGCACTTCTTCT |
| | | ACATCCACTACGCCTTCCCCACGTTCTTCAAGAACGTC |
| | | TTCGAAGGGCACATCGCGGAGCAGTACCTCAACACCCG |
| | | GGGCCGGCCGGACAAGGGCATGGCGACGCAGTACGGCC |
| | | TGGAGTCGACCCTCGAGTCGTACGCGGCCTACTACGGC |
| | | CCCTCCTACATGATCAATCCGCTCAAGAACAACTACGG |
| | | CGGGTACCAGACCGAATCCGTACTGATCAACTGCCATT |
| | | ACCCGATCACGCACGATTCGTTCATGCTGCAGTACGGC |
| | | ATCATCGTCAAGAAGCCGCAGGGCATGTCACCCGAGCA |
| | | GTCCGACGTGCTGGCCGCCAAGCTCACCGAGGGTGTCG |
| | | GTGAAGGCTTCCTGCAGGACGTCGAGATCTGGAAGAAC |
| | | AAGACCAAGATCGAGAATCCGCTGCTGTGCGAGGAGGA |
| | | TGGTCCGGTCTACCAGCTCCGTCGCTGGTACGAGCAGT |
| | | TCTACGTCGACGTCGCCGACGTGACGGAGAAGATGACG |
| | | GGCCGCTTCGAGTTCGAGGTCGACACCGCCAAGGCCAA |
| | | CGAGGCCTGGGAGAAGGAGGTCGCCGAGAATCTCGAGC |
| | | GCAAGAAGCGCGAGGAAGAACAGGGCAAGCAGGAAGCG |
| | | GAGGTGTGA |
| kshA4 Rhodococcus | HQ425876.1 | ATGACCGTCCCTCAGGAGCGGATCGAGATCCGCAACAT |
| rhodochrous (SEQ ID NO: 4) | | CGATCCCGGTACCAATCCCACCCGCTTCGCGCGCGGAT |
| | | GGCACTGCATCGGCCTCGCCAAGGATTTCCGCGACGGA |
| | | AAGCCGCACCAGGTCAAGGTGTTCGGCACCGACCTAGT |
| | | GGTCTTCGCCGACACGGCCGGAAAGTTGCACGTGCTCG |
| | | ACGCCTTCTGCCGGCACATGGGCGGCAACCTCGCTCGC |
| | | GGCGAGATCAAGGGCGACACCATCGCGTGCCCGTTCCA |
| | | CGACTGGCGCTGGAACGGCCAGGGCCGTTGCGAAGCGG |
| | | TGCCGTACGCGCGCCGCACGCCGAAGCTCGGCCGTACC |
| | | AAGGCGTGGACGACGATGGAGCGCAACGGCGTTCTGTT |
| | | CGTCTGGCACTGCCCGCAGGGTAGTGAGCCCACTCCCG |
| | | AGCTCGCGATCCCCGAGATCGAGGGCTACGAGGACGGG |
| | | CAGTGGAGCGACTGGACGTGGACGACTATCCACGTCGA |
| | | AGGATCGCACTGCCGCGAGATCGTCGACAACGTCGTCG |
| | | ACATGGCGCACTTCTTCTACGTGCACTTCCAGATGCCC |
| | | GAGTACTTCAAGAACGTCTTCGACGGGCACATCGCCGG |
| | | CCAGCACATGCGCTCCTACGGGCGCGACGACATCAAGA |
| | | CCGGTGTGCAGATGGACCTTCCGGAGGCGCAGACCATC |
| | | TCGOATGCCTTOTACTACGGTCCGTOCTTCATGOTCGA |
| | | CACCATCTACACGGTCTCCGAAGGCACGACCATCGAGT |
| | | CGAAGCTGATCAACTGCCACTACCCGGTCACGAACAAC |
| | | TCGTTCGTGCTGCAGTTCGGCACCATCGTCAAGAAGAT |
| | | CGAGGGCATGTCCGAGGAGCAGGCCGCGGAGATGGCGA |
| | | CGATGTTCACCGACGGTCTCGAGGAGCAGTTCGCCCAG |
| | | GACATCGAGATCTGGAAGCACAAGTCCCGCATCGAGAA |
| | | TCCGCTCCTCACCGAGGAGGACGGCCCGGTCTACCAGC |
| | | TGCGTCGCTGGTACAACCAGTTCTACGTCGACCTCGAG |
| | | GACGTCACACCGGACATGACCCAGCGTTTCGAGTTCGA |
| | | GGTGGACACCTCCCGTGCGCTCGAGTCGTGGCACAAGG |
| | | AGGTCGAGGAAAACCTCGCCGGTACGGCGGAGTGA |
| kshA5 Rhodococcus | HQ425877.1 | ATGTCCATCGACACCGCACGGTCCGGTTCGGACGACGA |
| rhodochrous (SEQ ID NO: 5) | | CGTCGAGATCCGCGAGATCCAGGCTGCGGCCGCTCCCA |
| | | CCCGCTTCGCACGGGGCTGGCACTGCCTCGGCCTGCTC |
| | | CGAGACTTCCAGGACGGCAAGCCGCACTCCATCGAGGC |
| | | CTTCGGAACCAAGCTGGTCGTGTTCGCCGACAGCAAGG |
| | | GGCAGCTCAACGTCCTCGATGCCTACTGCCGGCACATG |
| | | GGTGGCGACCTGAGCCGCGGCGAGGTCAAGGGCGACTC |
| | | GATCGCGTGCCCGTTCCACGACTGGCGCTGGAACGGCA |
| | | AGGGCAAGTGCACCGACATCCCCTACGCCCGGCGCGTC |
| | | CCGCCGATCGCGAAGACCCGCGCCTGGACGACCCTCGA |
| | | ACGCAACGGCCAGCTGTACGTCTGGAACGACCCGCAGG |
| | | GCAATCCGCCGCCGGAGGATGTCACCATCCCGGAGATC |
| | | GCCGGTTACGGCACCGACGAGTGGACGGACTGGAGCTG |
| | | GAAGAGCCTGCGCATCAAGGGCTCCCACTGCCGTGAGA |
| | | TCGTCGACAACGTCGTCGACATGGCGCACTTCTTCTAC |
| | | ATCCACTACTCGTTCCCGCGCTACTTCAAGAACGTCTT |
| | | CGAGGGCCACACCGCCACGCAGTACATGCACTCGACCG |
| | | GTCGTGAGGACGTCATCTCCGGCACCAACTACGACGAC |
| | | CCCAACGCCGAACTGCGTTCCGAGGCAACCTATTTCGG |
| | | TCCGTCGTACATGATCGACTGGCTCGAATCCGATGCCA |
| | | ACGGCCAGACCATCGAGACCATCCTCATCAACTGCCAC |
| | | TACCCGGTGAGCAACAACGAGTTCGTGCTGCAGTACGG |
| | | CGCGATCGTCAAGAAGCTCCCGGGGGTGTCGGACGAGA |
| | | TCGCCGCCGGGATGGCCGAGCAGTTCGCCGAGGGCGTG |
| | | CAGCTCGGTTTCGAGCAGGACGTCGAGATCTGGAAGAA |
| | | CAAGGCACCCATCGACAATCCGCTGCTGTCCGAGGAGG |
| | | ACGGCCCGGTCTACCAGCTGCGTCGCTGGTACCAGCAG |
| | | TTCTACGTCGATGTCGAGGACATCACCGAGGACATGAC |
| | | CAAGCGCTTCGAGTTCGAGATCGACACCACCCGGGCGG |
| | | TCGCGAGCTGGCAGAAGGAGGTCGCGGAGAACCTCGCG |
| | | AAGCAGGCCGAAGGCTCCACCGCGACCCCCTAG |
| kstD1 Rhodococcus | N/A | ATGGCGGAGTGGGCGGAAGAATGTGACGTCCTCGTGGT |
| rhodochrous (SEQ ID NO: 6) | | GGGGTCGGGAGCCGGAGGGTGCTGCGGTGCGTACACCG |
| | | CTGCGCGCGAAGGGCTGTCGGTGATCCTCGTCGAGGCG |
| | | TCCGAGTACTTCGGCGGCACCACGGCGTACTCCGGGGG |
| | | CGGCGGCGTCTGGTTCCCCACCAACGCGGTCCTGCAGC |
| | | GCGCCGGTGACGATGACACCATCGAGGATGCGCTGACC |
| | | TACTACCACGCGGTCGTCGGCGACCGCACCCCGCACGA |
| | | GCTGCAGGAGGCCTACGTTCGCGGCGGCGCCCCGCTGA |
| | | TCGACTACCTCGAGTCCGACGACGACCTCGAATTCATG |
| | | GTGTACCCGTGGCCCGACTACTTCGGCAAGGCGCCCAA |
| | | GGCCCGTGCCCAGGGACGGCACATCGTCCCGTCGCCGC |
| | | TGCCCATCGCCGGCGATCCCGAGCTCAACGAGTCGATC |
| | | CGCGGCCCGCTCGGCCGTGAACGCATCGGCGAACCCCT |
| | | GCCCGACATGCTCATCGGCGGTCGTGCGCTCGTCGGAC |
| | | GATTCCTCATCGCCCTGCGCAAGTACCCGAACGTGGAC |
| | | CTGTACCGGAACACCCCGCTCGAGGAACTGATCGTCGA |
| | | GGACGGCGTGGTCGTGGGCGCGGTCGTCGGGAACGACG |
| | | GTGAGCGACGTGCGATCCGCGCGCGCAAGGGCGTCGTC |
| | | CTGGCCGCCGGCGGTTTCGATCAGAACGACGAGATGCG |
| | | CGGCAAGTACGGGGTACCGGGTGCCGCGCGGGACTCGA |
| | | TGGGACCGTGGTCGAACCTCGGCAAGGCCCACGAGGCG |
| | | GGCATCGCCGTCGGCGCCGACGTGGATCTGATGGATCA |
| | | GGCCTGGTGGTCACCGGGACTGACCCATCCGGACGGAC |
| | | GCTCGGCGTTCGCGCTGTGCTTCACGGGCGGCATCTTC |
| | | GTCGACCAGGACGGTGCGCGGTTCACCAACGAGTACGC |
| | | ACCCTACGACCGTCTGGGCCGCGACGTCATCGCCCGCA |
| | | TGGAGCGCGGCGAGATGACGTTGCCGTTCTGGATGATC |
| | | TACGACGACCGGAACGGTGAGGCCCCGCCGGTCGGGGC |
| | | GACGAACGTGCCGCTCGTCGAGACCGAGAAGTACGTCG |
| | | ACGCGGGACTGTGGAAGACCGCCGACACCCTCGAGGAG |
| | | CTCGCCGGGCAGATCGGTGTGCCCGCCGAATCCCTGAA |
| | | GGCGACCGTCGCGCGGTGGAACGAGCTGGCCGCGAAGG |
| | | GAGTCGACGAAGACTTCGGTCGCGGGGACGAACCCTAC |
| | | GATCTCGCCTTCACCGGCGGTGGGTCCGCGCTGGTCCC |
| | | GATCGAGCAGGGCCCCTTCCACGCGGCGCAGTTCGGCA |
| | | TCTCCGATCTCGGCACCAAGGGCGGTCTGCGGACCGAC |
| | | ACCGTCGGGCGCGTGCTCGACAGCGAGGGTGCTCCGAT |
| | | CCCCGGTCTGTACGCGGCGGGCAACACGATGGCAGCAC |
| | | CGAGCGGCACCGTCTACCCCGGCGGTGGCAACCCGATC |
| | | GGCGCGAGCGCGCTGTTCGCGCACCTGTCCGTGATGGA |
| | | CGCTGCGGGACGCTGA |
| kstD2 Rhodococcus | N/A | ATGGCCAAGACCCCTGTACCGGCCGTGACCACAGCCCG |
| rhodochrous (SEQ ID NO: 7) | | CGATACGACCGTGGACCTGCTCGTGATCGGGTCCGGTA |
| | | CCGGCATGGCCGCTGCGCTCACCGCGCACGAGGCGGGC |
| | | CTGTCCGCTCTCATCGTGGAGAAGTCGGCCTACGTCGG |
| | | CGGATCGACCGCCCGTTCCGGCGGTGCATTCTGGGTGC |
| | | CGGCCAATCCGGTACTCACCGCGGCGGGAAGCGGCGAC |
| | | ACCATCGAGCGCGGCCACACCTACGTGCGGACGGTCGT |
| | | CGACGGCACGGCGCCGGTCGAGCGGGGCGAGGCCTTCG |
| | | TCGACAACGGTGTCGCCACCATCGAGATGCTCCAGCGC |
| | | ACCACCCCCATGAAGCTGTTCTGGGCCGAGGGCTACTC |
| | | CGACTATCACCCCGAACTGGCGGGTGGTTCGGCGGTCG |
| | | GCCGCAGCTGCGAGTGCCTGCCCCTCGACCTGTCGGTC |
| | | CTCGGTGAGGAGCGCGGTCGACTGCGTCCGGGCCTCAT |
| | | GGAGGCGAGCCTGCCGATGCCCACCACCGGTGCCGACT |
| | | ACAAGTGGATGAACCTCATGCTGCGCGTGCCGCACAAG |
| | | GGTTTTCCGCGCATCTTCAAGCGGCTCGCCCAGGGTGT |
| | | CGCCGGTCTCGCCGTCAAGCGTGAATATGTCGCGGGTG |
| | | GACAGGCGATCGCCGCCGGTCTGTTCGCGGGTGTGCTG |
| | | AAGGCCGGTGTCCCGGTGTGGACCGAGACGTCGCTGGT |
| | | GCGTCTGCTCACCGACGGGGACCGTGTCACCGGTGCCG |
| | | TCGTCGAGCAGAACGGACGTGAGGTGACGGTGACCGCG |
| | | CGTCGCGGGGTGGTGCTCGCCGCCGGCGGTTTCGACCA |
| | | CGACATGGAGATGCGGCGCAAGTTCCAGTCCGAGCGTC |
| | | TGCTCGACCACGAGAGCCTGGGAGCGGAGACCAACACC |
| | | GGCGACGCGATCAAGGCGGCCCAGGAGGTCGGTGCAGA |
| | | TCTCGCCCTCATGGACCAGGCCTGGTGGTTCCCTGCCG |
| | | TCGCGCCGACCCGCACGGGAAAGCCGCCGATGGTCATG |
| | | CTCGCCGAGCGGTCGCTGCCGGGTTCGTTCATCGTCGA |
| | | CCAGACGGGCCGCCGGTTCACCAACGAGTCGTCGGACT |
| | | ACATGTCGTTCGGACAGTTGGTGCTCGAACGTGAGCGT |
| | | GCCGGCGATCCGATCGAGTCGATGTGGATCGTCTTCGA |
| | | CCAGAAGTACCGCAACAGCTACGTCTTCGCGGCCGGGG |
| | | TGTTCCCGCGTCAACCGCTCCCGGAAGCCTGGTACGAG |
| | | GOGGGCATCGCCCACCGTGGCACCACCGCTGCGGAACT |
| | | CGCGGCGTCGATGGGCGTGCCGGTGGACACCTTCGCCG |
| | | CGACGTTCGACAGGTTCAACGAGGACGCGGCGGCGGGA |
| | | ACGGATTCCGAGTTCGGACGCGGCGGCAGTGCCTACGA |
| | | CCGCTACTACGGTGATCCGACCGTCCAGCCGAACCCGA |
| | | ACCTGCGGCCCCTCACGCACGGCCCGCTCTACGCGGTG |
| | | AAGATGACGCTGAGCGATCTCGGCACGTGCGGTGGCGT |
| | | GCGCGCCGACGAGCGGGCGCGGGTCCTCCGCGAGGACG |
| | | GCAGCCCCATCGCCGGTCTCTACGCTATCGGCAACACC |
| | | GCGGCCAACGCGTTCGGCCACCGCTATCCCGGTGCCGG |
| | | CGCCACGATCGGCCAGGGCCTGGTCTTCGGGTACATCG |
| | | CGGCACGCGACGCAGCATCGTCGGACGCACCGGTCGCC |
| | | TGA |
| kstD3 Rhodococcus | HQ425875.1 | ATGACGAAGCAGGAGTACGACATCGTTGTCGTCGGCAG |
| rhodochrous (SEQ ID NO: 8) | | CGGTGCCGGCGGAATGACCGCCGCCATCACCGCAGCCC |
| | | GCAAGGGCGCCGACGTGGTCCTGATCGAGAAGGCGCCA |
| | | CGCTACGGCGGGTCGAGCGCCCGATCGGGCGGCGGTGT |
| | | GTGGATCCCCAACAACGAGGCCCTGAAGGCCGCCGGGG |
| | | TGGACGACACACCCGAGGAGGCCCGGAAATACCTCCAC |
| | | AGCATCATCGGCGACGACGTACCCGCCGAGAAGATCGA |
| | | CACCTACATCGATCGCGGACCGGAGATGCTCTCCTTCG |
| | | TCCTGAAGAACAGCGCACTCGAACTGCAGTGGGTGCCG |
| | | GGCTATTCCGACTACTACCCCGAGGCGCCGGGCGGACG |
| | | TCCCGGTGGCCGTTCGGTGGAACCGACACCCTTCGACG |
| | | GTCGCCGTCTCGGCGAGGATCTCGCTCTCCTCGAACCC |
| | | GACTACGCCCGCGCTCCCAAGAACTTCGTCATCACCCA |
| | | GGCCGACTACAAGTGGCTGAACCTGCTCATGCGGAACC |
| | | CGCGCGGACCGATTCGCGCCATGCGGGTCGGCGCCCGG |
| | | TTCGTCTGGGCGAACATCACCAAGAAGCACCTGCTCGT |
| | | CCGAGGCCAGGCACTCATGGCCGGTCTGCGGATCGGTC |
| | | TGCGTGACGCCGGTGTGCCCCTGCTGCTGGAGACGGCG |
| | | CTCACCGACCTCGTCGTCGAGGGCGGCGCCGTGCGCGG |
| | | CGTCAAGGTGGTCGCGAACGGCGAGACGCGCGTCATCC |
| | | GTGCCCGCAAGGGCGTGATCATCGCGAGCGGOGGTTTC |
| | | GAGCACAACGCCGAGATGCGGGCGCAATACCAGCGTCA |
| | | GCCGATCGGCACCGAGTGGACCGTGGGGGCGAAGGCGA |
| | | ACACCGGCGACGGAATCCGCGCCGGACAGAAGCTGGGC |
| | | GCCGCAGTCGATTTCATGGACGACGCCTGGTGGGGACC |
| | | GTCCTTCACCCTCACCGGCGGCCCGTGGTTCGCACTGT |
| | | CGGAACGCAGCCTCCCCGGGTGCCTCATGGTCAACGCC |
| | | GOGGGCAAGCGTTTCGTCAACGAGTCGGCGCCCTACGT |
| | | CGAAGCGACGCATGCGATGTACGGCGGCAAGCACGGAC |
| | | GCGGCGAGGGACCGGGCGAGAACATCCCCAGCTGGCTG |
| | | ATCCTCGATCAGCGCTACCGCGACCGCTACACCTTCGC |
| | | CGGCATCACCCCCCGCACTCCCTTCCCCCGCCGGTGGC |
| | | TCGAGGCCGGGGTGOTCGTCAAGGCCGGTTCCGTCGCC |
| | | GAACTCGCCGAGAAGATCGGGGTACCGGCCGACGCCCT |
| | | CACCGAGACGGTGCAGCGGTTCAACGGCTTCGCCCGGG |
| | | CCGGCAAGGACGAGGACTTCGGCCGCGGCGAATCCCAC |
| | | TATGACCACTACTACGGGGATCCGCGCAACAAGCCGAA |
| | | TCCGAGCCTCGGCGTGGTCGATAAGGCCCCGTTCTACG |
| | | CGTTCAAGGTGGTCCCCGGCGATCTCGGCACCAAGGGC |
| | | GGGCTCGTCACCGACGTCCACGGCCGGGTGGTGCGCGA |
| | | GGACGGCAGCGTGATCGACGGCCTGTACGCGACCGGTA |
| | | ACGCCAGCTCCCCGGTCATGGGTCACACCTACGCCGGG |
| | | CCCGGTGCCACCATCGGACCGGCGATGACCTTCGGCTA |
| | | TCTCGCGGCCCTCGACATCCTGGATCGCACGGGTGACG |
| | | AACGCACCGAGGAACTGCGAGAATCCGCCGACACCGTG |
| | | TGA |
| fadE34 Rhodococcus | N/A | GTGAGTATCGCCACGACCGAGGAGCAGCGGGCCGTCCA |
| rhodochrous (SEQ ID NO: 9) | | GGCGTCTGTCCAGGCCTGGTCACGTGCCGTAGACCCCA |
| | | TGTCGACGATACGTCGCGCAGGTGATGCGACGTGGCGC |
| | | GACGGCTGGTCCTCCCTCGCAGAACTCGGAATCTTCGG |
| | | TGTTGCCGTCCCGGAGGAGGCGGGCGGCCTCGGCGCGA |
| | | CCGCCGTGGATCTGGCCGTCATGCTCGAGCAGGCCGCC |
| | | CACGAACTCGCGCCGGGTCCGGTCCTGACCACCGCCGT |
| | | GGCGGCCCTCGTGTTCGGCCGTGCCGGTGAGACCGTCG |
| | | CCAAGACGGCGGAGCGACTCGCCGAGGGTGAGGTCCCC |
| | | ACCGCACTCGCTCTCGACTCCGGCGTGACCGTGGAGCC |
| | | GGCGGGTGACGGAGTCCTGCTGCGCGGTGAGGCCGGGC |
| | | CGGCCGTGGGTGCCGAAGCCGGGGTCGCCGTGCTCGTC |
| | | CGTGTCGCGGGGGAAGGTGATCCGGCCGTCGAGAGCTG |
| | | GGCGCTCGTCGAGGCGGACGATCCGGGTCTGCACATCG |
| | | AACCGCTCGAGACCATCGACGCCTCCCGCGCGGTGGCC |
| | | CGCGTCCGCCTCGACGGCGCGACGGTCCCGGCCGACCG |
| | | GGTCGCGACCGTCCCGGCCGGCTTCGTGCGCGACCTCA |
| | | CCGCCGGTCTCGCCGCCGCGGAGCTGGCCGGTCTCGCC |
| | | GGTTGGGCGCTGACCACCGCCGTCGAGTACGCGAAGAT |
| | | CCGCGAGCAGTTCGGAAAACCGATCGGTTCGTTCCAGG |
| | | CCGTCAAGCACATCTGTGCCGAAATGCTCTGCCGCACC |
| | | GAGAAGATCCGGGCCATGGCCTGGGATGCTGCGGTCAC |
| | | CGTCGACGCGCAGCCCGACGAACTGCCGATCGCCGCGG |
| | | CTGCCGCCGTGGCGGTCGCACTCGATGCCGCGGTGCAG |
| | | ACCGCCAAGGATGCGATCCAGGTGCTCGGCGGCATCGG |
| | | GTTCACGTGGGAACACGACGCGCACTTCTATCTTCGCC |
| | | GTGCGGTCGCCACCCGCCAGGTGCTCGGTGGTTCGACC |
| | | GTGTGGCGTTCGCGGCTGACGACCCTGGTCCGCGCAGG |
| | | CGCACGTCGTCACCTCGGTATCGACCTGTCCGATCACG |
| | | AGGAGGAGCGCGCACGGATCCGTGCGGAAGTCGAGAAG |
| | | ATCGCCGCCGCACCGGAATCCGAGCGCCGCGTCGCCCT |
| | | CGCCGAGTCGGGTCTGCTCGCGCCGCACTGGCCGCAGC |
| | | CGTACGGTCGCGGAGCCGGTGCCGCCGAACAGCTCGTC |
| | | GTCCAGGAGGAGCTCGCCGCCGCCGGTATCGAACGTCC |
| | | CGATCTCGTGATCGGCTGGTGGGCGGTTCCGACTATCC |
| | | TCGAACACGGAACACCCGAGCAGATCGAGCGTTTCGTG |
| | | ATGCCCACCCTGCGCGGCGATGTGGTGTGGTGCCAGCT |
| | | CTTCTCCGAGCCCGGCGCCGGCTCGGACCTCGCGGCGC |
| | | TGCGCACGAGCGCGGAGAAGGCCGACGGCGGATGGGTG |
| | | CTGCGCGGGCAGAAGGTGTGGACCTCCCTCGCGCAGCA |
| | | GGCGGACTGGGCGATCTGCCTCGCCCGCACCGACCGCG |
| | | ACGTCCCCAAGCACAAGGGCATCACCTATTTCCTCGTC |
| | | GACATGAAGTCGGCGGGCATCACGATCTCGCCGCTGCG |
| | | CGAGATCACCGGCGACGCGTTGTTCAACGAGGTCTTCC |
| | | TCGATTCGGTCTTCGTGCCGGACGACTGCGTGGTCGGC |
| | | AATCTCGGTGACGGCTGGAAGCTGGCCCGCACGACTCT |
| | | CGCCAACGAGCGTGTCGCGATGGGCGGCAAGTCGTCGC |
| | | TGGGGCAGAGCATCGAGGAACTGCTCGAACTGTCGACC |
| | | CCCGGTGATCCCGTCGCAGAGGACCGCATCGCGACGCA |
| | | GATCGGCGAGGCGACCGTCGGTTCGCTCCTGGATCTGC |
| | | GGGCGACCCTCGCGCAGCTCGAAGGTCAGGATCCGGGC |
| | | GCCGCGTCCAGCGTCCGCAAGCTCATCGGTGTGCGGCA |
| | | GCGGCAGGACACCGCCGAGCTCGCCATGGATCTCGCGG |
| | | GCGAGGCCGGCTGGGTGGAAGGTCCGCTCACCCGGGAG |
| | | TTCCTCAACACCCGGTGCCTGACGATCGCCGGCGGGAC |
| | | CGAGCAGATCCTGCTCACCGTGGCGGCCGAGCGGCTGC |
| | | TGGGCCTGCCGCGGGGTTGA |
| fadE34#2 Rhodococcus | N/A | ATGACTCTGGGATTGAGCGACGAGGACCGCGAACTCCG |
| rhodochrous (SEQ ID NO: | | CGACTCCGTGCGCGGCTGGGCGGCACGACACGCCACAC |
| 10) | | CCGACGTGATCCGCACGGCCGTCGAAGCGAAGACGGAA |
| | | GCCCGCCCGACGTACTGGAGCTCGTTCGCCGAACTCGG |
| | | CATGCTGGGATTGCACCTGCCCGAAGAGGTCGGAGGCG |
| | | CCGGTTTCGGTCTGCTCGAAACGGCGATCGTCGCAGAG |
| | | GAACTCGGACGGGCCATGGTGCCCGGCCCGTTCCTTCC |
| | | GACCGTGATCGTGTCCGCGGTCCTCGACGAGGCCGGCC |
| | | GTCGCAGCGAACTCGACGGGCTCGCGGACGGTTCGCTG |
| | | TTCGGTGCGGTCGCCCTGCAGCCGGGGGACCTGCGCGT |
| | | GGAGCGCGACGGCGATTCCGTCACGCTCTCGGGAACCT |
| | | CCGGTGTCGCTCTCGGCGGCCAGGTCGCGGATGTCTTC |
| | | CTGCTCGCGGCCGACGACGGTGGTGAGCGGGTATTCGT |
| | | CGTCGTGACCCGTGACCGGGTCGAGGTCACGAACCTGC |
| | | CCAGCTACGACGTGATCCGCCGCAACGCCGAGATCACC |
| | | GTGAGTGCCGTGCCGCTGTCCGACGGGGACGTGCTGGA |
| | | GTCGGATCCGCATCGGATCGTCGATATCGCCGCGACCT |
| | | TGTTCGCCGCCGAAGCCGCCGGTCTCGCGGACTGGGCC |
| | | ACCACCACCGCCGCGGACTATGCGCGGGTCCGCAAGCA |
| | | GTTCGGCCGCGTCATCGGACAGTTCCAGGGTGTCAAGC |
| | | ACACCGTCGCCCGGATGCTCTGCCTCACCGAACAGGCG |
| | | CGGGTCGTGGCCTGGGACGCCGCGCGAGCGCGGCGCGA |
| | | GGACGTGCCGGACGACGAGGCGTCGCTGGCCGTGGCGG |
| | | TCGCCGCGTCCATCGCCCCCGAGGCCGCCTTCCAGGTC |
| | | ACCAAGAACTGCATCCAGGTGCTCGGCGGTATCGGCTA |
| | | CACCTGGGAGCACGACGCCCACCTGTACATGCGCCGCG |
| | | CCCAGTCGCTCCGAATCCTGCTCGGCTCCACGGCGTCC |
| | | TGGCGGCGCCGGGTCGCCCACCTCACGCTCGGCGGTGC |
| | | CCGCCGCGTGCTGAGCGTCGATCTGCCGCCCGAGGCGG |
| | | AACGGATCCGCGCCGACGTCCGTGCCGAACTCGAGCCG |
| | | GCGAAGTCGCTGGAGAACGCAGCGCGGAAGGCGTATCT |
| | | GGCGGAGAAGGGTTACACCGCTCCCCATCTGCCCGAAC |
| | | CGTGGGGCAAGGCCGCCGACGCCGTCACGCAACTCGTC |
| | | GTCGCCGAGGAACTGCGCGCCGCCGAACTCGAACCGCA |
| | | CGACATGATCATCGGCAACTGGGTGGTGCCGACCCTCA |
| | | TCGCGCACGGCAGTACCGAGCAGATCGAGCGATTCGTC |
| | | CCGCAGTCGCTGCGCGGGGATCTCGTGTGGTGTCAGCT |
| | | CTTCTCCGAACCCGGCGCCGGATCCGACCTCGCGGGCC |
| | | TGTCCACCAAGGCCGTCAAGGTGGACGGCGGATGGAGG |
| | | CTCGACGGCCAGAAGGTGTGGACGTCGATGGCACGGGT |
| | | CGCGGATTGGGGCATCTGCCTCGCCCGCACCGACGCGG |
| | | AAGCGCCCAAACACAAAGGCCTGTCCTACTTCCTGATC |
| | | GACATCAGGAACACCGAGGGTCTCGACATCCGGCCGCT |
| | | GCGAGAGATCACCGGCGAAGCCCTGTTCAACGAGGTGT |
| | | TCCTCGACGGCGTGTTCGTGCCCGACGAGTGCCTCGTC |
| | | GGCGAGCCCGGGGACGGATGGAAGCTCGCCCGTACCAC |
| | | CCTCGCGAACGAACGCGTCTCCCTCTCGCACGATTCGA |
| | | CTTTCGGTGCCGGCTGCGAGACTCTCATAGCGCTCGCG |
| | | AACGGTATGCCCGGTGGACCGGACGACGAACAACTCAC |
| | | CGTCCTCGGCAAGGTTCTOGGCGATGCCGCGTCCGGTG |
| | | GCCTCATGGGTCTGCGTACCGCTCTACGGTCCCTGGCC |
| | | GGCGCACAGCCGGGTGCCGAGTCCTCCGTCGCCAAGCT |
| | | CCTCGGCGTCGAGCACCTCCAGCAGGTCTGGGAGACCG |
| | | CGATGGACTGGGCCGGTACTGCGTCGTTGCTCGACGAC |
| | | CAGGACCGAACTTCGGCGACCCACATGTTCCTCAACGT |
| | | GCAGTGCATGTCCATCGCCGGTGGGACGACCAACGTCC |
| | | AGCTGAACATCATCGGTGAGCGGCTTCTCGGCCTGCCC |
| | | CGCGATCCCGAACCCGGAAAGTGA |
| fadE26 Rhodococcus | HM588720.1 | GTGGACATCTCCTACACCCCCGGGCAACAAGCCCTCCG |
| rhodochrous (SEQ ID NO: | | CGAGGAATTGCGGGCCTATTTCGCACAGATCATGACCC |
| 11) | | CCGAGCGCCGCGAGGCGCTCGCGGCCACGACCGGGGAG |
| | | TACGGCTCCGGCAACGTGTACCGCGAGGTCGTGCAGCA |
| | | GATGGGCAAGGACGGCTGGCTCACCCTCGGGTGGCCCG |
| | | AGGAATACGGCGGCCAGAACCGTTCCGCGATGGACCAA |
| | | TTGATCTTCACCGACGAGGCGGCCATCGCCGGCGCGCC |
| | | CGTCCCGTTCCTCACCATCGACTCGGTCGCGCCGACGA |
| | | TCATGCACTACGGCACGGACGAGCAGAAGGAGTTCTTC |
| | | CTCCCCCGCATCTCCGCGGGAGAACTGCACTTCTCGAT |
| | | CGGCTATTCCGAACCCGGCGCCGGCACCGACCTCGCCT |
| | | CGCTGCGCACCACCGCCGTGCGCGACGGCGACGAGTGG |
| | | GTCATCAACGGGCAGAAGATGTGGACGAGCCTGATCGC |
| | | CTACGCCGACTACGTCTGGCTCGCCGCGCGCACCAACC |
| | | CGGATGTCAAGAAGCACAAGGGGATCAGCGTCTTCATC |
| | | GTGCCGACCGACGCTCCCGGCTTCTCGTACACCCCCGT |
| | | GCACACCATGGCCGGCCCCGACACGAGCGCCACCTACT |
| | | ACCAGGACGTGCGCGTCCCGGCGTCCGCGCTCGTCGGT |
| | | GAGGTCGACGGCGGCTGGGCGCTCATCACCAACCAGCT |
| | | CAATCACGAGCGGGTCGCACTCACCTCCGCCGGTCCCG |
| | | TGCGCACCGCGCTGACCGAGGTCCGGCGCTGGGCGCAG |
| | | GAGACGCACCTGCCCGACGGACGACGGGTGATCGACCA |
| | | GGAATGGGTGCAGATCAACCTGGCACGCGTCCATGCCA |
| | | AGGCCGAATACCTGCAGCTGATGAACTGGGACATCGCC |
| | | TCGAGCGCCGGCACGACCCCGCTCGGTCCGGAGGCCGC |
| | | CTCGGCCAACAAGGTGTTCGGCACCGAATTCGCGACCG |
| | | AGGCCTACCGGTTGCTCATGGAGGTCCTCGGACCCGCG |
| | | GCGACGGTACGGCAGAACTCGGCCGGCGCACTGCTCCG |
| | | CGGCCGGATCGAACGCATGCACCGCAGTTCCCTCATCC |
| | | TCACCTTCGGTGGCGGCACCAACGAGGTCCAGCGCGAC |
| | | ATCATCGCGATGACCGCTCTCGGCCAGCCGCCCGCCAA |
| | | GCGTTAG |
| fadE34 Mycobacterium | N/A - full | GTGTCTGTGCTGTCCGTCCCGACCGATACATCGGATGA |
| neoaurum (SEQ ID NO: 12) | Mycobacterium | GGCCGCGGCCCGTGAACTGGTCAGAGACTGGGTTCCGA |
| | neoaurum | GCTCTGGGTCGATCACCGCGATCCGCAACGTCGAACTC |
| | genome | GGCGATCCGCAGGCCTGGCGCACGCCGTTTGCCGGCTT |
| | (CP011022.1) | CGCCGAACTAGGGGTATTCGGCGTCGCGGTGCCCGAGG |
| | | AGTACGGCGGGGCCGGCAGCACGGTGGCGGATCTGCTC |
| | | GCGATGATCGACGAGGCGGCCGCCGGCCTGATCCCGGG |
| | | ACCCGTCGCGGGGACCGCACTTGCCACCCTCGTCGCCG |
| | | ATGATCCGGCCGTCCTGGAGGCGTTGGCCACCGGGGAG |
| | | CGCAGCGCCGGGATCGCCATGACGTCCGACATCACGGT |
| | | CGATTCCGGTACCGCCACCGGCACCGCGCCCCACGTGC |
| | | TGGGTGCCGATCCCGGCGGGGTCCTCATCCTGCCTGCC |
| | | GGGCAGCATTGGATCCTGGTGGACGCGAGTTCCGACGG |
| | | GGTGACCATCGACCCGCTGGAGGCCACCGACTTCTCCC |
| | | GACCGCTGGCCCGGGTGACGCTGACATCGGCACCGGCG |
| | | CAGCAGCTGAATGCCTCGGCGCAGCGGGTCACCGACCT |
| | | GATGGCGACTGTGCTGGCGGCCGAGCTGGCCGGGTTGT |
| | | CGCGCTGGCTGCTCAACACCGCCAACGAGTACGCCAAG |
| | | GTGCGCGAACAGTTCGGCAAGCCGATCGGCAGCTTCCA |
| | | GGCCGTCAAACACATGTGCGCGGAGATGCTGCTGCGTA |
| | | GCCAGCAGGTCACCGTCGCCGCCGCCGACGCGATCGCG |
| | | GCCGCTGCCGGTGACGACGCCGACCAGCTGTCCGTCGC |
| | | CGCGGCGGTGGCGGCGGCCATCGGTATCGACGCCGCGA |
| | | AGCTGAACGCGCGCGACTGCATCCAGGTGCTCGGCGGG |
| | | ATCGGCATCACCTGGGAGCACGATGCGCACCTGTACCT |
| | | GCGTCGGGCATATGCGAACGCGCAGTTCCTCGGTGGCC |
| | | GGTCGCGTTGGTTGCGTCGCGTCGTCGAACTGACCCGT |
| | | GCCGGCGTGCGCCGCGAACTGCACGTCGACACCGCTGA |
| | | TGCCGATGCCATCCGTCCCGAGATCGCCGCGGCCGCCG |
| | | CCCGCATCGCCGCGCTGCCCGAGGACCAACGAGGGCGG |
| | | GCACTCGCCGAATCCGGGCTGCTGGCCCCGCATTGGCC |
| | | GACGCCGTACGGGCGGGACGCGACCCCGGCCGAACAGT |
| | | TGGTGATCGACGAGGAACTGGCGGCTGCCGAGGTGGCG |
| | | CGCCCCGATATCTCGATCGGCTGGTGGGCCGCTCCGAC |
| | | GATCCTTGCCGCCGGTACGCCCGAACAGATCGATCGGT |
| | | TCATCCCCGGCACCCTCAACGGCGACATCTTCTGGTGC |
| | | CAGCTGTTCTCCGAGCCCGGCGCGGGGTCGGATCTGGC |
| | | GGCGTTGCGCACCAAGGCCGTTCGTGTGGAGAAGGATG |
| | | GCCGCACTGGCTGGTCTCTGACCGGACAGAAGGTGTGG |
| | | ACCTCCAACGCGCACCGCGCCAACTGGGGCATCTGCCT |
| | | GGCCCGGACCAACCCGGACGCTCCGAAACACAAGGGCA |
| | | TCTCCTATTTCCTGGTCGATATGAGCTCACCGGGTATC |
| | | GATATCCGGCCGCTGCGCGAGATCACCGGTGAGGCCCT |
| | | GTTCAACGAGGTCTTCTTCGATGACCTGTTCGTTCCCG |
| | | ACGACTGCGTGGTCGGTGAGGTGGACGGTGGCTGGCCG |
| | | CTGGCCCGTACCACGCTGGCCAACGAGCGCGTCGCCAT |
| | | CGCCACCGGCGGGGCACTGGACAAGGGCATGGAGCATC |
| | | TGCTTGCCGTGATCGGTGACCGGGAGCTCGACGGCGCC |
| | | GAGGCCGATCGGCTCGGTGCCCTGATCACCCTGGCCCA |
| | | GGTCGGTTCGCTGCTGGATCAGCTCATCGCGCGGATGG |
| | | CGTTGGGCGGCAATGATCCTGGTGCTCCGTCGAGCGTG |
| | | CGCAAGCTGATCGGCGTGCGTTATCGACAGGGGTTGGC |
| | | CGAGGCGGCGATGGAGTTCCAGGACGGTGGCGGCATCG |
| | | TCGACTCGCCCGATGTCCGGTACTTCCTCAACACCCGC |
| | | TGCTTGAGCATCGCCGGGGGCACCGAGCAGATCCTGCT |
| | | CACCCTCGCCGGTGAGCGGCTGCTGGGGTTGCCGCGCT |
| | | AG |

APPENDIX B
Amino acid sequences

| | GENBANK | |
|---|---|---|
| Name and SEQ ID NO. | Accession No. | Amino acid sequence |
| kshA1 Rhodococcus | ADY18310.1 | VSLGTSEQSEIREIVAGSAPARFARGWHCLGLAKDFKD |
| rhodochrous (SEQ ID NO: | | GKPHSVHAFGTKLVVWADSNDEIRILDAYCRHMGGDLS |
| 13) | | QGTVKGDEIACPFHDWRWGGNGRCKNIPYARRVPPIAK |
| | | TRAWHTLDQDGLLFVWHDPQGNPPPADVTIPRIAGATS |
| | | DEWTDWVWYTTEVDTNCREIIDNIVDMAHFFYVHYSFP |
| | | VYFKNVFEGHVASQFMRGQAREDTRPHANGQPKMIGSR |
| | | SDASYFGPSFMIDDLVYEYEGYDVESVLINCHYPVSQD |
| | | KFVLMYGMIVKKSDRLEGEKALQTAQQFGNFIAKGFEQ |
| | | DIEIWRNKTRIDNPLLCEEDGPVYQLRRWYEQFYVDVE |
| | | DVAPEMTDRFEFEMDTTRPVAAWMKEVEANIARKAALD |
| | | TETRSAPEQSTTAG |
| kshA2 Rhodococcus | ADY18316.1 | VGSTDTEDQVRTIDVGIPPERYARGWHCLGLVRDFADG |
| rhodochrous (SEQ ID NO: | | KPHQVDAFGTSLVVFAGEDGKLNVLDAYCRHMGGNLAQ |
| 14) | | GSVKGNTIACPFHDWRWRGDGKCAEIPYARRVPPLART |
| | | RTWPVAEVSGQLFVWHDPQGSKPPAELAVPEVPTYGDP |
| | | GWTDWVWNSIEVTGSHCREIVDNVVDMAHFFYVHYGMP |
| | | TYFRNVFEGHTATQVMRSLPRADAVGVSQATNYSAESR |
| | | SDATYYGPSYMIDKLWSAGRDPESTPNIYLINCHYPIS |
| | | PTSFRLQYGVMVERPEGVPPEQAEQIAQAVAQGVAIGF |
| | | EQDVEIWKNKSRIDNPLLCEEDGPVYQLRRWYEQFYVD |
| | | VEDIRPEMVNRFEYEIDTTRALTSWQAEVDENVAAGRS |
| | | AFAPNLTRAREAASAESGS |
| kshA3 Rhodococcus | ADY18318.l | MAQIREIDVGEVRTRFARGWHCLGLSRTFKDGKPHAVE |
| rhodochrous (SEQ ID NO: | | AFGTKLVVWADSNGEPKVLDAYCRHMGGDLSQGEIKGD |
| 15) | | SVACPFHDWRWGGNGKCTDIPYARRVPPLARTRSWITM |
| | | EKHGQLFVWNDPEGNIPPPEVTIPEIEQYGSDEWTDWT |
| | | WNQIRIEGSNCREIIDNVVDMAHFFYIHYAFPTFEKNV |
| | | FEGHIAEQYLNTRGRPDKGMATQYGLESTLESYAAYYG |
| | | PSYMINPLKNNYGGYQTESVLINCHYPITHDSFMLQYG |
| | | IIVKKPQGMSPEQSDVLAAKLTEGVGEGFLQDVEIWKN |
| | | KTKIENPLLCEEDGPVYQLRRWYEQFYVDVADVTEKMT |
| | | GRFEFEVDTAKANEAWEKEVAENLERKKREEEQGKQEA |
| | | EV |
| kshA4 Rhodococcus | ADY18323.1 | MTVPQERIEIRNIDPGINPTRFARGWHCIGLAKDFRDG |
| rhodochrous (SEQ ID NO: | | KPHQVKVFGTDLVVFADTAGKLHVLDAFCRHMGGNLAR |
| 16) | | GEIKGDTIACPFHDWRWNGQGRCEAVPYARRTPKLGRT |
| | | KAWTTMERNGVLFVWHCPQGSEPTPELAIPEIEGYEDG |
| | | QWSDWTWTTIHVEGSHCREIVDNVVDMAHFFYVHFQMP |
| | | EYFKNVFDGHIAGQHMRSYGRDDIKTGVQMDLPEAQTI |
| | | SDAFYYGPSFMLDTIYTVSEGTTIESKLINCHYPVTNN |
| | | SFVLQFGTIVKKIEGMSEEQAAEMATMFTDGLEEQFAQ |
| | | DIEIWKHKSRIENPLLTEEDGPVYQLRRWYNQFYVDLE |
| | | DVTPDMTQRFEFEVDTSRALESWHKEVEENLAGTAE |
| kshA5 Rhodococcus | ADY18328.1 | MSIDTARSGSDDDVEIREIQAAAAPTRFARGWHCLGLL |
| rhodochrous (SEQ ID NO: | | RDFQDGKPHSIEAFGTKLVVFADSKGQLNVLDAYCRHM |
| 17) | | GGDLSRGEVKGDSIACPFHDWRWNGKGKCTDIPYARRV |
| | | PPIAKTRAWTTLERNGQLYVWNDPQGNPPPEDVTIPEI |
| | | AGYGTDEWTDWSWKSLRIKGSHCREIVDNVVDMARFFY |
| | | IHYSFPRYFKNVFEGHTATQYMHSTGREDVISGTNYDD |
| | | PNAELRSEATYFGPSYMIDWLESDANGQTIETILINCH |
| | | YPVSNNEFVLQYGAIVKKLPGVSDEIAAGMAEQFAEGV |
| | | QLGFEQDVEIWKNKAPIDNPLLSEEDGPVYQLRRWYQQ |
| | | FYVDVEDITEDMTKRFEFEIDTTRAVASWQKEVAENLA |
| | | KQAEGSTATP |
| kstD1 Rhodococcus | N/A | MAEWAEECDVLVVGSGAGGCCGAYTAAREGLSVILVEA |
| rhodochrous (SEQ ID NO: | | SEYFGGTTAYSGGGGVWFPTNAVLQRAGDDDTIEDALT |
| 18) | | YYHAVVGDRTPHELQEAYVRGGAPLIDYLESDDDLEFM |
| | | VYPWPDYFGKAPKARAQGRHIVPSPLPIAGDPELNESI |
| | | RGPLGRERIGEPLPDMLIGGRALVGRFLIALRKYPNVD |
| | | LYRNTPLEELIVEDGVVVGAVVGNDGERRAIRARKGVV |
| | | LAAGGFDQNDEMRGKYGVPGAARDSMGPWSNLGKAHEA |
| | | GIAVGADVDLMDQAWWSPGLTHPDGRSAFALCFTGGIF |
| | | VDQDGARFTNEYAPYDRLGRDVIARMERGEMTLPFWMI |
| | | YDDRNGEAPPVGATNVPLVETEKYVDAGLWKTADTLEE |
| | | LAGQIGVPAESLKATVARWNELAAKGVDEDFGRGDEPY |
| | | DLAFTGGGSALVPIEQGPFHAAQFGISDLGTKGGLRTD |
| | | TVGRVLDSEGAPIPGLYAAGNTMAAPSGTVYPGGGNPI |
| | | GASALFAHLSVMDAAGR |
| kstD2 Rhodococcus | N/A | MAKTPVPAVTTARDTTVDLLVIGSGTGMAAALTAHEAG |
| rhodochrous (SEQ ID NO: | | LSALIVEKSAYVGGSTARSGGAFWVPANPVLTAAGSGD |
| 19) | | TIERGHTYVRTVVDGTAPVERGEAFVDNGVATIEMLQR |
| | | TTPMKLFWAEGYSDYHPELAGGSAVGRSCECLPLDLSV |
| | | LGEERGRLRPGLMEASLPMPTTGADYKWMNLMLRVPHK |
| | | GFPRIFKRLAQGVAGLAVKREYVAGGQATAAGLFAGVL |
| | | KAGVPVWTETSLVRLLTDGDRVTGAVVEQNGREVTVTA |
| | | RRGVVLAAGGFDHDMEMRRKFQSERLLDHESLGAETNT |
| | | GDAIKAAQEVGADLALMDQAWWFPAVAPTRTGKPPMVM |
| | | LAERSLPGSFIVDQTGRRFTNESSGYMSFGQLVLERER |
| | | AGDPIESMWIVFDQKYRNSYVFAAGVFPRQPLPEAWYE |
| | | AGIAHRGTTAAELAASMGVPVDTFAATFDRFNEDAAAG |
| | | TDSEFGRGGSAYDRYYGDPTVQPNPNLRPLTHGPLYAV |
| | | KMTLSDLGTCGGVRADERARVLREDGSPIAGLYAIGNT |
| | | AANAFGHRYPGAGATIGQGLVFGYIAARDAASSDAPVA |
| kstD3 Rhodococcus | ADY18320.1 | MTKQEYDIVVVGSGAGGMTAAITAARKGADVVLIEKAP |
| rhodochrous (SEQ ID NO: | | RYGGSSARSGGGVWIPNNEALKAAGVDDTPEEARKYLH |
| 20) | | SIIGDDVPAEKIDTYIDRGPEMLSFVLKNSALELQWVP |
| | | GYSDYYPEAPGGRPGGRSVEPTPFDGRRLGEDLALLEP |
| | | DYAPAPKNFVITQADYKWLNLLMRNPRGPIRAMRVGAR |
| | | FVWANITKKHLLVRGQALMAGLRIGLRDAGVPLLLETA |
| | | LTDLVVEGGAVRGVKVVANGETRVIRARKGVIIASGGF |
| | | EHNAEMRAQYQRQPIGTEWTVGAKANTGDGIRAGQKLG |
| | | AAVDFMDDAWWGPSFTLTGGPWFALSERSLPGCLMVNA |
| | | AGKRFVNESAPYVEATHAMYGGKHGRGEGPGENIPSWL |
| | | ILDQRYRDRYTFAGITPRTPFPRRWLEAGVLVKAGSVA |
| | | ELAEKIGVPADALTETVQRFNGFARAGKDEDFGRGESH |
| | | YDHYYGDPRNKPNPSLGVVDKAPFYAFKVVPGDLGTKG |
| | | GLVTDVHGRVVREDGSVIDGLYATGNASSPVMGHTYAG |
| | | PGATIGPAMTFGYLAALDILDRTGDERTEELRESADTV |
| fadE34 Rhodococcus | N/A | VSIATTEEQRAVQASVQAWSRAVDPMSTIRRAGDATWR |
| rhodochrous (SEQ ID NO: | | DGWSSLAELGIFGVAVPEEAGGLGATAVDLAVMLEQAA |
| 21) | | HELAPGPVLTTAVAALVFGRAGETVAKTAERLAEGEVP |
| | | TALALDSGVTVEPAGDGVLLRGEAGPAVGAEAGVAVLV |
| | | RVAGEGDPAVESWALVEADDPGLHIEPLETIDASRAVA |
| | | RVRLDGATVPADRVATVPAGFVRDLTAGLAAAELAGLA |
| | | GWALTTAVEYAKIREQFGKPIGSFQAVKHICAEMLCRT |
| | | EKIRAMAWDAAVTVDAQPDELPIAAAAAVAVALDAAVQ |
| | | TAKDAIQVLGGIGFTWEHDAHFYLRRAVATRQVLGGST |
| | | VWRSRLTTLVRAGARRHLGIDLSDHEEERARIRAEVEK |
| | | IAAAPESERRVALAESGLLAPHWPQPYGRGAGAAEQLV |
| | | VQEELAAAGIERPDLVIGWWAVPTILEHGTPEQIERFV |
| | | MPTLRGDVVWCQLFSEPGAGSDLAALRTSAEKADGGWV |
| | | LRGQKVWTSLAQQADWAICLARTDRDVPKHKGITYFLV |
| | | DMKSAGITISPLREITGDALFNEVFLDSVFVPDDCVVG |
| | | NLGDGWKLARTTLANERVAMGGKSSLGQSIEELLELST |
| | | PGDPVAEDRIATQIGEATVGSLLDLRATLAQLEGQDPG |
| | | ASSVRKLIGVRQRQDTAELAMDLAGEAGWVEGPLTRE |
| | | FLNTRCLTIAGGTEQILLTVAAERLLGLPRG |
| fadE34#2 Rhodococcus | N/A | MTLGLSDEDRELRDSVRGWAARHATPDVIRTAVEAKTE |
| rhodochrous (SEQ ID NO: | | ARPTYWSSFAELGMLGLHLPEEVGGAGFGLLETAIVAE |
| 22) | | ELGRAMVPGPFLPTVIVSAVLDEAGRRSELDGLADGSL |
| | | FGAVALQPGDLRVERDGDSVTLSGTSGVALGGQVADVF |
| | | LLAADDGGERVFVVVTRDRVEVTNLPSYDVIRRNAEIT |
| | | VSAVPLSDGDVLESDPHRIVDIAATLFAAEAAGLADWA |
| | | TTTAADYARVRKQFGRVIGQFQGVKHTVARMLCLTEQA |
| | | RVVAWDAARARREDVPDDEASLAVAVAASIAPEAAFQV |
| | | TKNCIQVLGGIGYTWEHDAHLYMRRAQSLRILLGSTAS |
| | | WRRRVAHLTLGGARRVLSVDLPPEAERIRADVRAELEP |
| | | AKSLENAARKAYLAEKGYTAPHLPEPWGKAADAVTQLV |
| | | VAEELRAAELEPHDMIIGNWVVPTLIAHGSTEQIERFV |
| | | PQSLRGDLVWCQLFSEPGAGSDLAGLSTKAVKVDGGWR |
| | | LDGQKVWTSMARVADWGICLARTDAEAPKHKGLSYFLI |
| | | DIRNTEGLDIRPLREITGEALFNEVFLDGVFVPDECLV |
| | | GEPGDGWKLARTTLANERVSLSHDSTFGAGCETLIALA |
| | | NGMPGGPDDEQLTVLGKVLGDAASGGLMGLRTALRSLA |
| | | GAQPGAESSVAKLLGVEHLQQVWETAMDWAGTASLLDD |
| | | QDRTSATHMFLNVQCMSIAGGTTNVQLNIIGERLLGLP |
| | | RDPEPGE |
| fadE26 Rhodococcus | ADP09632.1 | MDISYTPGQQALREELRAYFAQIMTPERREALAATTGE |
| rhodochrous (SEQ ID NO: | | YGSGNVYREVVQQMGKDGWLTLGWPEEYGGQNRSAMDQ |
| 23) | | LIFTDEAAIAGAPVPFLTIDSVAPTIMHYGTDEQKEFF |
| | | LPRISAGELHFSIGYSEPGAGTDLASLRTTAVRDGDEW |
| | | VINGQKMWTSLIAYADYVWLAARTNPDVKKHKGISVFI |
| | | VPTDAPGFSYTPVHTMAGPDTSATYYQDVRVPASALVG |
| | | EVDGGWALITNQLNHERVALTSAGPVRTALTEVRRWAQ |
| | | ETHLPDGRRVIDQEWVQINLARVHAKAEYLQLMNWDIA |
| | | SSAGTTPLGPEAASANKVFGTEFATEAYRLLMEVLGPA |
| | | ATVRQNSAGALLRGRIERMHRSSLILTFGGGTNEVQRD |
| | | AMTALGQPPAKR |
| fadE34 Mycobacterium | N/A | VSVLSVPTDTSDEAAARELVRDWVPSSGSITAIRNVEL |
| neoaurum (SEQ ID NO: 24) | | GDPQAWRTPFAGFAELGVFGVAVPEEYGGAGSTVADLL |
| | | AMIDEAAAGLIPGPVAGTALATLVADDPAVLEALATGE |
| | | RSAGIAMTSDITVDSGTATGTAPHVLGADPGGVLILPA |
| | | GQHWILVDASSDGVTIDPLEATDFSRPLARVTLTSAPA |
| | | QQLNASAQRVTDLMATVLAAELAGLSRWLLNTANEYAK |
| | | VREQFGKPIGSFQAVKHMCAEMLLRSQQVTVAAADAIA |
| | | AAAGDDADQLSVAAAVAAAIGIDAAKLNARDCIQVLGG |
| | | IGITWEHDAHLYLRRAYANAQFLGGRSRWLRRVVELTR |
| | | AGVRRELHVDTADADAIRPEIAAAAARIAALPEDQRGR |
| | | ALAESGLLAPHWPTPYGRDATPAEQLVIDEELAAAEVA |
| | | RPDISIGWWAAPTILAAGTPEQIDRFIPGTLNGDIFWC |
| | | QLFSEPGAGSDLAALRTKAVRVEKDGRTGWSLTGQKVW |
| | | TSNAHRANWGICLARTNPDAPKHKGISYFLVDMSSPGI |
| | | DIRPLREITGEALFNEVFFDDLEVPDDCVVGEVDGGWP |
| | | LARTTLANERVAIATGGALDKGMEHLLAVIGDRELDGA |
| | | EADRLGALITLAQVGSLLDQLIARMALGGNDPGAPSSV |
| | | RKLIGVRYRQGLAEAAMEFQDGGGIVDSPDVRYFLNTR |
| | | CLSIAGGTEQILLTLAGERLLGLPR |

APPENDIX C
Strains and plasmids referred to in the Examples

| Strain | | | Reference |
|---|---|---|---|
| code | Full name | Strain description | |
| DH5α | E. coli DH5α | General host for cloning | Bethesda |
| | | | Research |
| | | | Laboratories |
| S17-1 | E. coli S17-1 | Host strain for conjugal mobilization of | DSMZ |
| | | pK18mobsacB-derived mutagenic | collection |
| | | plasmids to Rhodococcus strains | |
| WT | Rhodococcus rhodochrous | Wild-type strain | DS MZ |
| | DSM43269 | | collection |
| RG32 | WTΔkshA1ΔkshA2ΔkshA3Δ | 5-fold kshA null mutant in WT | Wilbrink et al |
| | kshA4ΔkshA5 | | 2011 |
| RG35 | RG32ΔkstD3 | Deletion of kstD3 in RG32 | This work |
| RG36 | RG32ΔkstD1ΔkstD3 | Deletion of kstD1 in RG35 | This work |
| RG41 | RG32ΔkstD1ΔkstD2ΔkstD3 | Deletion of kstD2 in RG36 kshA null + | This work |
| | | kstD1, 2 and 3 mutant | |
| LM3 | RG41ΔfadE34 | Deletion of fadE34 in RG41 | This work |
| | RG41ΔfadE34#2 | Deletion of fadE34#2 in RG41 | This work |
| LM9 | RG41ΔfadE34ΔfadE34#2 | Deletion of fadE34#2 in LM3 | This work |
| LM33 | RG41ΔfadE34ΔfadE34#2ΔfadE26 | Deletion of fadE26 in double mutant | This work |
| | | LM9 | |
| LM19 | RG41ΔfadE34ΔfadE34#2 | Complementation with kshA5 in LM9 | This work |
| | kshA5-complem | | |
| Mneo | Mycobacterium neoaurum NRRL | Parent strain | Marsheck et |
| | B-3805 | | al, 1972 |
| Mneo- | M. neoaurum NRRL B-3805- | Deletion of fadE34 in Mneo | This work |
| ΔfadE34 | ΔfadE34 | | |
| | Plasmid | Description | Reference |
| | pBluescrip(II)KS | General cloning vector | Stratagene |
| | pZErO-2.1 | General cloning vector | Invitrogene |
| | pk18mobsacB | Conjugative plasmid for gene | Gene (1994) |
| | | mutagenesis in Rhodococcus; aphll | 145: 69 |
| | | sacB oriT (RP4) lacZ | |
| | pKSH800 | Clone isolated from genomic library of | Wilbrink et al, |
| | | WT strain carrying kshA3 and kstD3 | 2011 |
| | pKSH841 | pK18mobsacB-derived mutagenic | This work |
| | | plasmid for deletion of kstD3 in RG32 | |
| | pKSH852 | pK18mobsacB-derived mutagenic | This work |
| | | plasmid for deletion of kstD1 in RG35 | |
| | pKSD321 | clone isolated from genomic library of | This work |
| | | RG36 strain carrying kstD2 | |
| | pKSD26 | pK18mobsacB-derived mutagenic | This work |
| | | plasmid for deletion of kstD2 in RG36 | |
| | pK18 + fadE34-UP + DON | pK18mobsacB-derived mutagenic | This work |
| | | plasmid for deletion of fadE34 in RG41 | |
| | pK18 + fadE2-UP + DOWN | pK18mobsacB-derived mutagenic | This work |
| | | plasmid for deletion of fadE34#2 in | |
| | | RG41 and LM3 | |
| | pDEfadE26 | pK18mobsacB-derived mutagenic | Wilbrink et al |
| | | plasmid for deletion of fadE26 in LM9 | 2011 |
| | pK18 + kshA5-complementation | pK18mobsacB-derived mutagenic | This work |
| | | plasmid for complementation with | |
| | | kshA5 in LM9 | |
| | pK18 + fadE34_Mneo-UP + | pK18mobsacB-derived mutagenic | This work |
| | DOWN | plasmid for deletion of fadE34 in Mneo | |

APPENDIX D
Primers referred to in the Examples

| Target | | | | |
|---|---|---|---|---|
| Gene | PCR amplicon | Size | Primer name | Primer sequence (5′-3′) |
| kstD1 | Construction and | WT: 2.4 kb/ | kstD1-F | TGGCAGCAGAACTCGCCGGG |
| | checking deletion | ΔkstD1: | | (SEQ ID NO: 25) |
| | kstD1 | 1.3 kb | kstD1-R | CCGGAACGACACCGATGCGCCG |
| | | | | (SEQ ID NO: 26) |
| kstD2 | Construction and | WT: 0.8 kb/ | kstD2-F | CTACAGCGACTACCACCCCGATTT |
| | checking deletion | ΔkstD2: no | | (SEQ ID NO: 27) |
| | kstD2 | amplif | kstD2-R | CTGTTGCGGTACTTCTGGTCGAA |
| | | | | (SEQ ID NO: 28) |
| kstD3 | Checking deletion | WT: 2.9 kb/ | kstD3-F | CGACCTGTCACAGGGCGAGAT |
| | kstD3 | ΔkstD3: 2 kb | | (SEQ ID NO: 29) |
| | | | kstD3-R | GGACCACCTTGAACGCGTAGC |
| | | | | (SEQ ID NO: 30) |
| fadE34 | Upstream region for | 1.5 kb | FadE34-UP_F | GCGATAAGATCTTGGTGGCGGATG |
| | deletion fadE34 | | | ACGTCGAG (SEQ ID NO: 31) |
| | | | FadE34-UP_R | GCGATATCTAGAGGCCCGCTGCTC |
| | | | | CTCGGTC (SEQ ID NO: 32) |
| | Downstream region | 1.5 kb | FadE34-DOWN_F | GCGATATCTAGAATCGCCGGCGGG |
| | for deletion fadE34 | | | ACCGAG (SEQ ID NO: 33) |
| | | | FadE34- | GCGATAAAGCTTGCAGGAACTTCC |
| | | | DOWN_R | GCTTCT (SEQ ID NO: 34) |
| fadE34 | Upstream region for | 1.5 kb | FadE34#2-UP_F | GCGATAAGATCTCCTTCTGCTGGT |
| #2 | deletion fadE34#2 | | | CGATCTG (SEQ ID NO: 35) |
| | | | FadE34#2-UP_R | CGCTATTCTAGAGAGTTCGGCGAA |
| | | | | CGAGCTCC (SEQ ID NO: 36) |
| | Downstream for | 1.5 kb | Fad E34#2- | GCGATATCTAGATTGCTCGACGAC |
| | deletion region | | DOWN_F | CAGGACCGAACTTC (SEQ ID |
| | fadE34#2 | | | NO: 37) |
| | | | FadE34#2- | CGCTATAAGCTTAGCTGTGCGGTG |
| | | | DOWN_R | GCGCCGCTG (SEQ ID NO: 38) |
| fadE34 | Checking deletion | WT: 5.4 kb/ | Flanking_fadE34- | GAACGCGAGCGCGGCGATGACCTC |
| | fadE34 | ΔfadE34: | F | T (SEQ ID NO: 39) |
| | | 3.4 kb | Flanking_fadE34- | GGTCCAGCTGAAGCCGGGATCCTT |
| | | | R | G (SEQ ID NO: 40) |
| fadE34 | Checking deletion | WT 5.7 kb/ | Flanking_fadE34# | GAGGTCGCCGAACTCGCCGGTGTC |
| #2 | fadE34#2 | ΔfadE34#2: | 2_F | GCCATC (SEQ ID NO: 41) |
| | | 3.8 kb | Flanking_fadE34# | GCGTGCACCTGTTCGCGGTCGGTG |
| | | | 2_R | ACATCC (SEQ ID NO: 42) |
| kshA5 | Construction and | ΔkshA5: | kshA5-complem-F | GCGATAGGATCCGGCCCGGATTGT |
| | checking | 1.2 kb/ | | CGCTGATG (SEQ ID NO: 43) |
| | complementation | complemented: | kshA5-complem-R | CGCTATAAGCTTGATCACGTGCAG |
| | kshA5 | 2.2 kb | | CATGC (SEQ ID NO: 44) |
| fadE34_ | Upstream region for | 1.5 kb | FadE34_Mneo- | GCGATAGGATCCGACACCGACTTC |
| Mneo | deletion | | UP-F | CTGCTGTTG (SEQ ID NO: 45) |
| | fadE34_Mneo | | FadE34_Mneo- | CGCTATTCTAGACCGATGTCCGGT |
| | | | UP-R | ACTTCCTC (SEQ ID NO: 46) |
| | Downstream region | 1.5 kb | FadE34_Mneo- | GCGATATCTAGAGATCGCCGAGTT |
| | for deletion | | DOWN-F | CGACGTTG (SEQ ID NO: 47) |
| | fadE34_Mneo | | FadE34_Mneo- | CGCTATAAGCTTGTGACGATCACC |
| | | | DOWN-R | GCGAACTC (SEQ ID NO: 48) |
| fadE34_ | Checking deletion | parent: 2.5 kb/ | FadE34_Mneo-F | AGATTCGGTGCAGACCGATTG |
| Mneo | fadE34_Mneo | ΔfadE34: | | (SEQ ID NO: 49) |
| | | 0.5 kb | FadE34Mneo-R | AAGCTGCATGCGGATCCAC (SEQ |
| | | | | ID NO: 50) |

1. A genetically-modified bacterium blocked in a steroid metabolism pathway prior to degradation of a polycyclic steroid ring system, wherein the genetically-modified bacterium is disrupted in a steroid side-chain degradation pathway, and wherein the genetically-modified bacterium converts a steroidal substrate into a steroidal product of interest.

2. The genetically-modified bacterium of claim 1, wherein the disruption in the steroid side-chain degradation pathway occurs after the first cycle of β-oxidation.

3-8. (canceled)

9. The genetically-modified bacterium of claim 1, wherein the genetically-modified bacterium is of an Actinobacteria class or a Gammaproteobacteria class.

10. The genetically-modified bacterium of claim 9, wherein the genetically-modified bacterium of the Actinobacteria class is a Rhodococcus species, a Mycobacterium species, a Nocardia species, a Corynebacterium species, or an Arthrobacter species.

11. The genetically-modified bacterium of claim 10, wherein the Rhodococcus species is Rhodococcus rhodochrous, Rhodococcus erythropolis, Rhodococcus jostii, or Rhodococcus ruber.

12. The genetically-modified bacterium of claim 10, wherein the Mycobacterium species is Mycobacterium neoaurum, Mycobacterium smegmatis, Mycobacterium tuberculosis, or Mycobacterium fortuitum.

13. The genetically-modified bacterium of claim 10, wherein the Nocardia species is Nocardia restrictus, Nocardia corallina, or Nocardia opaca.

14. The genetically-modified bacterium of claim 10, wherein the Arthrobacter species is Arthrobacter simplex.

15. The genetically-modified bacterium of claim 1, wherein the genetic modification comprises inactivation of genes: kshA1 (SEQ ID NO: 1), kshA2 (SEQ ID NO: 2), kshA3 (SEQ ID NO: 3), kshA4 (SEQ ID NO: 4), and kshA5 (SEQ ID NO: 5), or a homologs thereof.

16. The genetically-modified bacterium of claim 15, wherein the genetic modification further comprises re-introduction of a wild type copy of the kshA5 gene comprising SEQ ID NO: 5, or a homolog thereof.

17. The genetically-modified bacterium of claim 1, wherein the genetic modifications comprise inactivation of genes: kshA1 (SEQ ID NO: 1), kshA2 (SEQ ID NO: 2), kshA3 (SEQ ID NO: 3), and kshA4 (SEQ ID NO: 4), or a homologs thereof.

18. The genetically-modified bacterium of claim 15, wherein the genetic modification further comprises inactivation of genes: kstD1 (SEQ ID NO: 6), kstD2 (SEQ ID NO: 7), and kstD3 (SEQ ID NO: 8), or a homologs thereof.

19. The genetically-modified bacterium of claim 1, wherein the genetic modification comprises inactivation of one or more of genes: fadE34 (SEQ ID NO: 9; SEQ ID NO: 12), fadE34#2 (SEQ ID NO: 10), or a homologs thereof.

20. The genetically-modified bacterium of claim 19, wherein the genetic modification further comprises inactivation of gene: fadE26 (SEQ ID NO: 11), or a homologs thereof.

21. The genetically-modified bacterium of claims 15 to 20, wherein the gene inactivation is by gene deletion.

22. The genetically-modified bacterium of claim 15, wherein the homolog has a nucleotide sequence with at least 50% sequence identity with the genes.

23. (canceled)

24. The genetically-modified bacterium of claim 15, wherein the homolog encodes a polypeptide that has an amino acid sequence with at least 50% sequence identity with the genes.

25. (canceled)

26. The genetically-modified bacterium of claim 1, which is a genetically-modified Rhodococcus rhodochrous bacterium of strain: LM9 (Accession No. NCIMB 43058), LM19 (Accession No. NCIMB 43059), or LM33 (Accession No. NCIMB 43060).

27. The genetically-modified bacterium of claim 1, which is a genetically-modified Mycobacterium neoaurum bacterium of strain: NRRL B-3805 Mneo-ΔfadE34 (Accession No. NCIMB 43057).

28. (canceled)

29. A method of converting a steroidal substrate into a steroidal product of interest, comprising the steps of:
(a) inoculating a culture medium with the genetically-modified bacteria according to claim 1 to prepare a bacterial culture, and growing the bacterial culture until a target OD₆₀₀ is reached;
(b) adding a steroidal substrate to the bacterial culture when the target OD₆₀₀ is reached;
(c) culturing the bacterial culture so that the steroidal substrate is converted to a steroidal product of interest; and,
(d) extracting and/or purifying the steroidal product of interest from the bacterial culture.

30. The method according to claim 29, wherein the culture medium is a LB medium or minimal medium.

31. The method according to claim 29, wherein in step (a) the bacterial culture is grown to a target OD₆₀₀ of at least 1.0.

32. The method according to claim 29, wherein the steroidal substrate is a sterol substrate selected from: 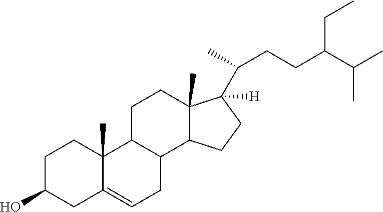 [SMILES: CCC(CC[C@@H](C)[C@H]1CCC2C3CC=C4C[C@@H](O)CC[C@]4(C)C3CC[C@@]21C)C(C)C]
β-sitosterol; 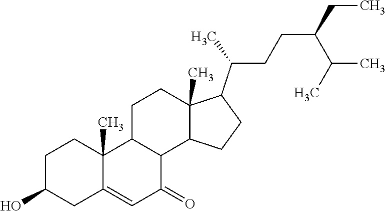 [SMILES: CC[C@H](CC[C@@H](C)C1CCC2C3C(=O)C=C4C[C@@H](O)CC[C@]4(C)C3CC[C@@]21C)C(C)C]
7-oxo-β-sitosterol or 7-hydroxy-β-sitosterol; 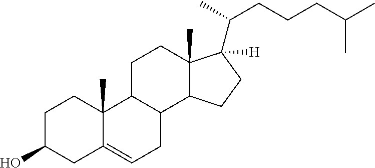 [SMILES: CC(C)CCC[C@@H](C)[C@H]1CCC2C3CC=C4C[C@@H](O)CC[C@]4(C)C3CC[C@@]21C]
cholesterol; 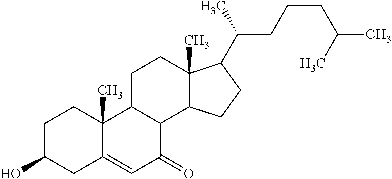 [SMILES: CC(C)CCC[C@@H](C)C1CCC2C3C(=O)C=C4C[C@@H](O)CC[C@]4(C)C3CC[C@@]21C]
7-oxo-cholesterol or 7-hydroxy-β-cholesterol; 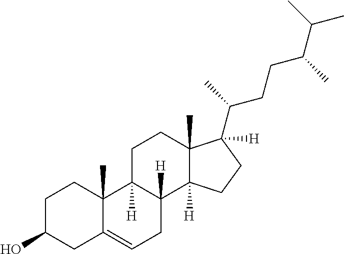
campesterol; 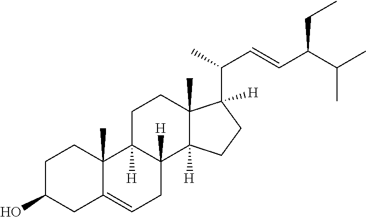
stigmasterol; 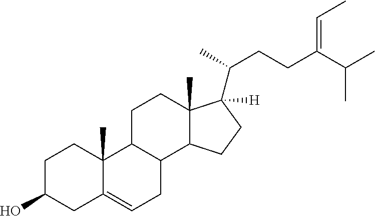 [SMILES: C/C=C(/CC[C@@H](C)[C@H]1CCC2C3CC=C4C[C@@H](O)CC[C@]4(C)C3CC[C@@]21C)C(C)C]
fucosterol;
7-oxo-phytosterol; or a combination thereof.

33-35. (canceled)

36. The method according to claim 29, wherein the steroidal product of interest is: 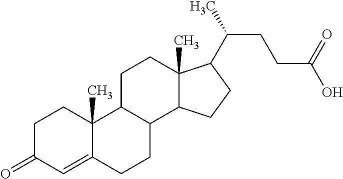 [SMILES: C[C@H](CCC(=O)O)C1CCC2C3CCC4=CC(=O)CC[C@]4(C)C3CC[C@@]21C]
3-oxo-4-cholenic acid; 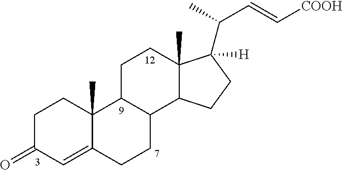 [SMILES: C[C@H](/C=C/C(=O)O)[C@H]1CCC2C3CCC4=CC(=O)CC[C@]4(C)C3CC[C@@]21C]
Chola 4,22-dien-24-oic acid, 3-oxo (CAS 59648-73-6, or CAS 82637-22-7 for pure E isomer); 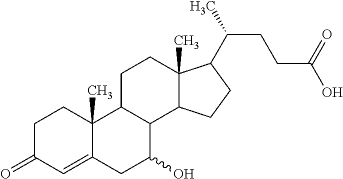 [SMILES: C[C@H](CCC(=O)O)C1CCC2C3C(O)CC4=CC(=O)CC[C@]4(C)C3CC[C@@]21C]
3-oxo-7-hydroxy-4-cholenic acid; 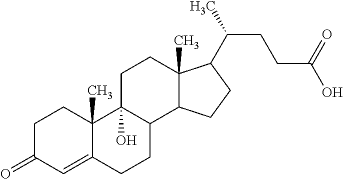
3-oxo-9-hydroxy-4-cholenic acid; 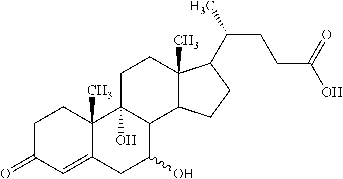
3-oxo-7,9-dihydroxy-4-cholenic acid; 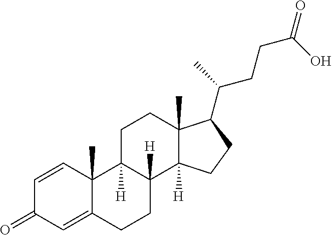
3-oxo-1,4-choladienoic acid; 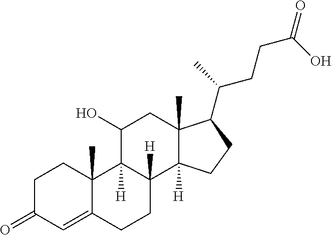
3-oxo-11-hydroxy-4-cholenic acid; 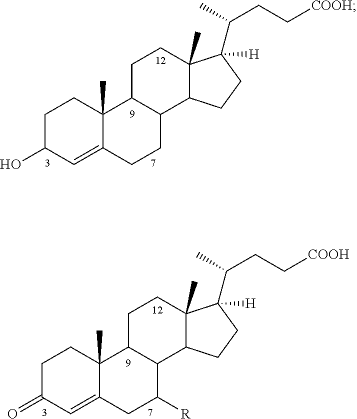
wherein R can be hydroxyl, oxo, or a halogen; 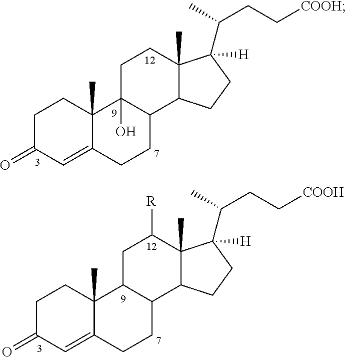
wherein R can be hydroxyl or oxo; 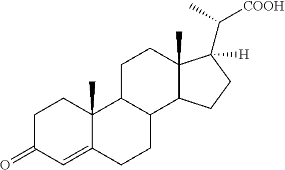 [SMILES: C[C@H](C(=O)O)[C@H]1CCC2C3CCC4=CC(=O)CC[C@]4(C)C3CC[C@@]21C]
3-oxo-23,24-bisnor-4-cholene-22-oic acid (4-BNC); 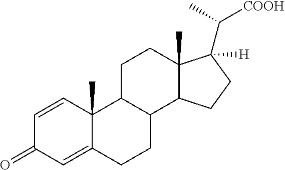 [SMILES: C[C@H](C(=O)O)[C@H]1CCC2C3CCC4=CC(=O)C=C[C@]4(C)C3CC[C@@]21C]
3-oxo-23,24-bisnor-1,4-choladiene-22-oic acid (1,4-BNC); or variants thereof.

37. (canceled)

38. The method of claim 29, wherein in step (b) the steroidal substrate is added at a concentration of at least 0.1 mM.

39-46. (canceled)

47. The method according to claim 29, wherein in step (b) a cyclodextrin and/or an organic solvent are added to the culture medium.

48. The method according to claim 47, wherein the cyclodextrin is added at concentration of 1 mM to 25 mM and the organic solvent is added at a volume/volume (v/v) concentration of 1% to 10%.

49. (canceled)

50. A steroidal product of interest produced by the method of claim 29.

51. A kit for converting a steroidal substrate into a steroidal product of interest, wherein the kit comprises:
(a) a genetically-modified bacterium according to claim 1; and,
(b) instructions for using the kit.

52-58. (canceled)


SEQUENCE LISTING (50 sequences)

SEQ ID NO: 1 - DNA, 1185 bp, Rhodococcus rhodochrous
  kshA1
gtgagcctcggcacttccgaacaatccgaaatccgtgagatcgtcgccgggtcggctcccgcccgcttcgcccgcggctggcactgcctcggcctggcgaaggatttcaaggacggcaagccgcattccgtgcacgccttcggtaccaaactcgtggtgtgggccgacagcaacgacgagatcaggatcctcgacgcgtactgccggcacatgggcggcgatctcagccagggcaccgtcaagggcgacgagatcgcgtgcccgttccacgactggcgctggggcggcaacggccgctgcaagaacatcccgtacgcacgtcgtgttcccccgatcgcgaagacccgcgcgtggcacacgctcgatcaggacgggctgctgttcgtctggcacgacccccagggcaatccgccgccggccgacgtgacgatcccgcgcatcgcgggtgcgacgagcgacgagtggaccgactgggtctggtacaccaccgaggtcgacaccaactgccgcgagatcatcgacaacatcgtcgacatggcgcacttcttctacgtgcactactccttcccggtgtacttcaagaacgtcttcgaaggacacgtcgccagccagttcatgcgcggtcaggcccgtgaggacacccgtccgcacgcgaacggtcaaccgaagatgatcggaagccgatccgatgcaagctatttcggcccgtccttcatgatcgacgatctcgtctacgagtacgagggatacgacgtcgagtcggtcctcatcaactgccactacccggtctcccaggacaagttcgtcctgatgtacggcatgatcgtcaagaagtccgaccgtctcgagggcgagaaggcgttgcagaccgcgcagcagttcggcaacttcatcgcgaagggtttcgagcaggacatcgagatctggcgcaacaagacccgcatcgacaacccgctcctgtgcgaggaggacggccccgtctaccagctgcgtcgctggtacgagcagttctacgtcgacgtcgaggacgtcgcgcccgagatgaccgaccgcttcgagttcgagatggacaccacccgtcccgtcgcggcgtggatgaaggaggtcgaggcgaacatcgcccgcaaggccgccctcgacacggaaactcgttctgcaccagagcagtccaccaccgcgggctag

SEQ ID NO: 2 - DNA, 1200 bp, Rhodococcus rhodochrous
  kshA2
gtgggttccacagacaccgaagatcaggtccgcaccatcgatgtgggcacgccgccggagcgctacgcgcgaggatggcactgcctggggctcgtacgcgatttcgccgacggcaagccccaccaggtcgacgcgttcgggacctcgctcgtggtgttcgccggtgaggacggaaagctcaacgttctggacgcctactgcaggcacatgggtggaaatctggcccagggatccgtgaagggcaacaccatcgcctgtccgttccacgactggcgctggcgcggtgacgggaagtgtgccgagattccctatgcgcgccgtgttccaccgctcgcccgtacccggacgtggccggtggcggaggtgagcggtcagctcttcgtgtggcacgacccgcagggcagcaagccgccggcggagctcgccgttccggaggttcccacctacggcgatcccgggtggaccgactgggtgtggaactcgatcgaggtgaccggatcccactgtcgcgagatcgtggacaacgtcgtcgacatggcgcactttttctacgtccactacgggatgccgacctacttccgaaacgtgttcgaaggtcatacggccacccaggtcatgcggtccctgccccgggcggacgccgtaggcgtcagccaggccaccaattacagtgccgagagcagatccgatgcaacgtattacggtccctcgtacatgatcgacaagctgtggagcgccggccgtgatcccgagtcgacgccgaacatctatctgatcaactgccactaccccatctctccgacctccttccgcctgcagtacggcgtgatggtggaaaggcccgagggagtgcccccggagcaggcggaacagatcgcccaggccgtcgcccagggcgtcgcgatcggattcgagcaggacgtcgagatctggaagaacaagtcgcggatcgacaaccccctgctgtgcgaggaggacggtcccgtctaccaactgcggcggtggtacgaacagttctacgtcgacgtcgaagacatccgacccgagatggtcaaccggttcgagtacgagatcgacaccacgcgcgccctgacgagctggcaggccgaagtcgacgagaacgtcgcggccggacgtagtgccttcgccccgaacctcacccgggctcgtgaagcagcctccgccgaatcgggatcctga

SEQ ID NO: 3 - DNA, 1149 bp, Rhodococcus rhodochrous
  kshA3
atggcacagattcgcgagatcgacgtcggagaggtccggacgcgtttcgcgcgaggctggcactgcctcggcctcagtcgcacgttcaaggacggcaagccccacgccgtcgaggccttcggcacgaaactcgtggtgtgggccgacagcaacggcgaaccgaaggtgctcgacgcgtactgccgtcacatgggcggcgacctgtcacagggcgagatcaagggcgattcggttgcgtgcccgttccacgactggcgctggggcggcaacggcaagtgcacggacatcccgtatgccaggcgcgttcccccgctggcccgcacccgttcgtggataacgatggagaagcacggccagctgttcgtgtggaacgaccccgagggcaacaccccgcccccggaggtcacgatccccgagatcgagcagtacggctcggacgagtggacggactggacctggaaccagatccggatcgaaggttccaactgtcgcgagatcatcgacaacgtcgtcgacatggcgcacttcttctacatccactacgccttccccacgttcttcaagaacgtcttcgaagggcacatcgcggagcagtacctcaacacccggggccggccggacaagggcatggcgacgcagtacggcctggagtcgaccctcgagtcgtacgcggcctactacggcccctcctacatgatcaatccgctcaagaacaactacggcgggtaccagaccgaatccgtactgatcaactgccattacccgatcacgcacgattcgttcatgctgcagtacggcatcatcgtcaagaagccgcagggcatgtcacccgagcagtccgacgtgctggccgccaagctcaccgagggtgtcggtgaaggcttcctgcaggacgtcgagatctggaagaacaagaccaagatcgagaatccgctgctgtgcgaggaggatggtccggtctaccagctccgtcgctggtacgagcagttctacgtcgacgtcgccgacgtgacggagaagatgacgggccgcttcgagttcgaggtcgacaccgccaaggccaacgaggcctgggagaaggaggtcgccgagaatctcgagcgcaagaagcgcgaggaagaacagggcaagcaggaagcggaggtgtga

SEQ ID NO: 4 - DNA, 1137 bp, Rhodococcus rhodochrous
  kshA4
atgaccgtccctcaggagcggatcgagatccgcaacatcgatcccggtaccaatcccacccgcttcgcgcgcggatggcactgcatcggcctcgccaaggatttccgcgacggaaagccgcaccaggtcaaggtgttcggcaccgacctagtggtcttcgccgacacggccggaaagttgcacgtgctcgacgccttctgccggcacatgggcggcaacctcgctcgcggcgagatcaagggcgacaccatcgcgtgcccgttccacgactggcgctggaacggccagggccgttgcgaagcggtgccgtacgcgcgccgcacgccgaagctcggccgtaccaaggcgtggacgacgatggagcgcaacggcgttctgttcgtctggcactgcccgcagggtagtgagcccactcccgagctcgcgatccccgagatcgagggctacgaggacgggcagtggagcgactggacgtggacgactatccacgtcgaaggatcgcactgccgcgagatcgtcgacaacgtcgtcgacatggcgcacttcttctacgtgcacttccagatgcccgagtacttcaagaacgtcttcgacgggcacatcgccggccagcacatgcgctcctacgggcgcgacgacatcaagaccggtgtgcagatggaccttccggaggcgcagaccatctcggatgccttctactacggtccgtccttcatgctcgacaccatctacacggtctccgaaggcacgaccatcgagtcgaagctgatcaactgccactacccggtcacgaacaactcgttcgtgctgcagttcggcaccatcgtcaagaagatcgagggcatgtccgaggagcaggccgcggagatggcgacgatgttcaccgacggtctcgaggagcagttcgcccaggacatcgagatctggaagcacaagtcccgcatcgagaatccgctcctcaccgaggaggacggcccggtctaccagctgcgtcgctggtacaaccagttctacgtcgacctcgaggacgtcacaccggacatgacccagcgtttcgagttcgaggtggacacctcccgtgcgctcgagtcgtggcacaaggaggtcgaggaaaacctcgccggtacggcggagtga

SEQ ID NO: 5 - DNA, 1173 bp, Rhodococcus rhodochrous
  kshA5
atgtccatcgacaccgcacggtccggttcggacgacgacgtcgagatccgcgagatccaggctgcggccgctcccacccgcttcgcacggggctggcactgcctcggcctgctccgagacttccaggacggcaagccgcactccatcgaggccttcggaaccaagctggtcgtgttcgccgacagcaaggggcagctcaacgtcctcgatgcctactgccggcacatgggtggcgacctgagccgcggcgaggtcaagggcgactcgatcgcgtgcccgttccacgactggcgctggaacggcaagggcaagtgcaccgacatcccctacgcccggcgcgtcccgccgatcgcgaagacccgcgcctggacgaccctcgaacgcaacggccagctgtacgtctggaacgacccgcagggcaatccgccgccggaggatgtcaccatcccggagatcgccggttacggcaccgacgagtggacggactggagctggaagagcctgcgcatcaagggctcccactgccgtgagatcgtcgacaacgtcgtcgacatggcgcacttcttctacatccactactcgttcccgcgctacttcaagaacgtcttcgagggccacaccgccacgcagtacatgcactcgaccggtcgtgaggacgtcatctccggcaccaactacgacgaccccaacgccgaactgcgttccgaggcaacctatttcggtccgtcgtacatgatcgactggctcgaatccgatgccaacggccagaccatcgagaccatcctcatcaactgccactacccggtgagcaacaacgagttcgtgctgcagtacggcgcgatcgtcaagaagctcccgggggtgtcggacgagatcgccgccgggatggccgagcagttcgccgagggcgtgcagctcggtttcgagcaggacgtcgagatctggaagaacaaggcacccatcgacaatccgctgctgtccgaggaggacggcccggtctaccagctgcgtcgctggtaccagcagttctacgtcgatgtcgaggacatcaccgaggacatgaccaagcgcttcgagttcgagatcgacaccacccgggcggtcgcgagctggcagaaggaggtcgcggagaacctcgcgaagcaggccgaaggctccaccgcgaccccctag

SEQ ID NO: 6 - DNA, 1536 bp, Rhodococcus rhodochrous
  kstD1
atggcggagtgggcggaagaatgtgacgtcctcgtggtggggtcgggagccggagggtgctgcggtgcgtacaccgctgcgcgcgaagggctgtcggtgatcctcgtcgaggcgtccgagtacttcggcggcaccacggcgtactccgggggcggcggcgtctggttccccaccaacgcggtcctgcagcgcgccggtgacgatgacaccatcgaggatgcgctgacctactaccacgcggtcgtcggcgaccgcaccccgcacgagctgcaggaggcctacgttcgcggcggcgccccgctgatcgactacctcgagtccgacgacgacctcgaattcatggtgtacccgtggcccgactacttcggcaaggcgcccaaggcccgtgcccagggacggcacatcgtcccgtcgccgctgcccatcgccggcgatcccgagctcaacgagtcgatccgcggcccgctcggccgtgaacgcatcggcgaacccctgcccgacatgctcatcggcggtcgtgcgctcgtcggacgattcctcatcgccctgcgcaagtacccgaacgtggacctgtaccggaacaccccgctcgaggaactgatcgtcgaggacggcgtggtcgtgggcgcggtcgtcgggaacgacggtgagcgacgtgcgatccgcgcgcgcaagggcgtcgtcctggccgccggcggtttcgatcagaacgacgagatgcgcggcaagtacggggtaccgggtgccgcgcgggactcgatgggaccgtggtcgaacctcggcaaggcccacgaggcgggcatcgccgtcggcgccgacgtggatctgatggatcaggcctggtggtcaccgggactgacccatccggacggacgctcggcgttcgcgctgtgcttcacgggcggcatcttcgtcgaccaggacggtgcgcggttcaccaacgagtacgcaccctacgaccgtctgggccgcgacgtcatcgcccgcatggagcgcggcgagatgacgttgccgttctggatgatctacgacgaccggaacggtgaggccccgccggtcggggcgacgaacgtgccgctcgtcgagaccgagaagtacgtcgacgcgggactgtggaagaccgccgacaccctcgaggagctcgccgggcagatcggtgtgcccgccgaatccctgaaggcgaccgtcgcgcggtggaacgagctggccgcgaagggagtcgacgaagacttcggtcgcggggacgaaccctacgatctcgccttcaccggcggtgggtccgcgctggtcccgatcgagcagggccccttccacgcggcgcagttcggcatctccgatctcggcaccaagggcggtctgcggaccgacaccgtcgggcgcgtgctcgacagcgagggtgctccgatccccggtctgtacgcggcgggcaacacgatggcagcaccgagcggcaccgtctaccccggcggtggcaacccgatcggcgcgagcgcgctgttcgcgcacctgtccgtgatggacgctgcgggacgctga

SEQ ID NO: 7 - DNA, 1713 bp, Rhodococcus rhodochrous
  kstD2
atggccaagacccctgtaccggccgtgaccacagcccgcgatacgaccgtggacctgctcgtgatcgggtccggtaccggcatggccgctgcgctcaccgcgcacgaggcgggcctgtccgctctcatcgtggagaagtcggcctacgtcggcggatcgaccgcccgttccggcggtgcattctgggtgccggccaatccggtactcaccgcggcgggaagcggcgacaccatcgagcgcggccacacctacgtgcggacggtcgtcgacggcacggcgccggtcgagcggggcgaggccttcgtcgacaacggtgtcgccaccatcgagatgctccagcgcaccacccccatgaagctgttctgggccgagggctactccgactatcaccccgaactggcgggtggttcggcggtcggccgcagctgcgagtgcctgcccctcgacctgtcggtcctcggtgaggagcgcggtcgactgcgtccgggcctcatggaggcgagcctgccgatgcccaccaccggtgccgactacaagtggatgaacctcatgctgcgcgtgccgcacaagggttttccgcgcatcttcaagcggctcgcccagggtgtcgccggtctcgccgtcaagcgtgaatatgtcgcgggtggacaggcgatcgccgccggtctgttcgcgggtgtgctgaaggccggtgtcccggtgtggaccgagacgtcgctggtgcgtctgctcaccgacggggaccgtgtcaccggtgccgtcgtcgagcagaacggacgtgaggtgacggtgaccgcgcgtcgcggggtggtgctcgccgccggcggtttcgaccacgacatggagatgcggcgcaagttccagtccgagcgtctgctcgaccacgagagcctgggagcggagaccaacaccggcgacgcgatcaaggcggcccaggaggtcggtgcagatctcgccctcatggaccaggcctggtggttccctgccgtcgcgccgacccgcacgggaaagccgccgatggtcatgctcgccgagcggtcgctgccgggttcgttcatcgtcgaccagacgggccgccggttcaccaacgagtcgtcggactacatgtcgttcggacagttggtgctcgaacgtgagcgtgccggcgatccgatcgagtcgatgtggatcgtcttcgaccagaagtaccgcaacagctacgtcttcgcggccggggtgttcccgcgtcaaccgctcccggaagcctggtacgaggcgggcatcgcccaccgtggcaccaccgctgcggaactcgcggcgtcgatgggcgtgccggtggacaccttcgccgcgacgttcgacaggttcaacgaggacgcggcggcgggaacggattccgagttcggacgcggcggcagtgcctacgaccgctactacggtgatccgaccgtccagccgaacccgaacctgcggcccctcacgcacggcccgctctacgcggtgaagatgacgctgagcgatctcggcacgtgcggtggcgtgcgcgccgacgagcgggcgcgggtcctccgcgaggacggcagccccatcgccggtctctacgctatcggcaacaccgcggccaacgcgttcggccaccgctatcccggtgccggcgccacgatcggccagggcctggtcttcgggtacatcgcggcacgcgacgcagcatcgtcggacgcaccggtcgcctga

SEQ ID NO: 8 - DNA, 1713 bp, Rhodococcus rhodochrous
  kstD3
atgacgaagcaggagtacgacatcgttgtcgtcggcagcggtgccggcggaatgaccgccgccatcaccgcagcccgcaagggcgccgacgtggtcctgatcgagaaggcgccacgctacggcgggtcgagcgcccgatcgggcggcggtgtgtggatccccaacaacgaggccctgaaggccgccggggtggacgacacacccgaggaggcccggaaatacctccacagcatcatcggcgacgacgtacccgccgagaagatcgacacctacatcgatcgcggaccggagatgctctccttcgtcctgaagaacagcgcactcgaactgcagtgggtgccgggctattccgactactaccccgaggcgccgggcggacgtcccggtggccgttcggtggaaccgacacccttcgacggtcgccgtctcggcgaggatctcgctctcctcgaacccgactacgcccgcgctcccaagaacttcgtcatcacccaggccgactacaagtggctgaacctgctcatgcggaacccgcgcggaccgattcgcgccatgcgggtcggcgcccggttcgtctgggcgaacatcaccaagaagcacctgctcgtccgaggccaggcactcatggccggtctgcggatcggtctgcgtgacgccggtgtgcccctgctgctggagacggcgctcaccgacctcgtcgtcgagggcggcgccgtgcgcggcgtcaaggtggtcgcgaacggcgagacgcgcgtcatccgtgcccgcaagggcgtgatcatcgcgagcggcggtttcgagcacaacgccgagatgcgggcgcaataccagcgtcagccgatcggcaccgagtggaccgtgggggcgaaggcgaacaccggcgacggaatccgcgccggacagaagctgggcgccgcagtcgatttcatggacgacgcctggtggggaccgtccttcaccctcaccggcggcccgtggttcgcactgtcggaacgcagcctccccgggtgcctcatggtcaacgccgcgggcaagcgtttcgtcaacgagtcggcgccctacgtcgaagcgacgcatgcgatgtacggcggcaagcacggacgcggcgagggaccgggcgagaacatccccagctggctgatcctcgatcagcgctaccgcgaccgctacaccttcgccggcatcaccccccgcactcccttcccccgccggtggctcgaggccggggtgctcgtcaaggccggttccgtcgccgaactcgccgagaagatcggggtaccggccgacgccctcaccgagacggtgcagcggttcaacggcttcgcccgggccggcaaggacgaggacttcggccgcggcgaatcccactatgaccactactacggggatccgcgcaacaagccgaatccgagcctcggcgtggtcgataaggccccgttctacgcgttcaaggtggtccccggcgatctcggcaccaagggcgggctcgtcaccgacgtccacggccgggtggtgcgcgaggacggcagcgtgatcgacggcctgtacgcgaccggtaacgccagctccccggtcatgggtcacacctacgccgggcccggtgccaccatcggaccggcgatgaccttcggctatctcgcggccctcgacatcctggatcgcacgggtgacgaacgcaccgaggaactgcgagaatccgccgacaccgtgtga

SEQ ID NO: 9 - DNA, 2148 bp, Rhodococcus rhodochrous
  fadE34
gtgagtatcgccacgaccgaggagcagcgggccgtccaggcgtctgtccaggcctggtcacgtgccgtagaccccatgtcgacgatacgtcgcgcaggtgatgcgacgtggcgcgacggctggtcctccctcgcagaactcggaatcttcggtgttgccgtcccggaggaggcgggcggcctcggcgcgaccgccgtggatctggccgtcatgctcgagcaggccgcccacgaactcgcgccgggtccggtcctgaccaccgccgtggcggccctcgtgttcggccgtgccggtgagaccgtcgccaagacggcggagcgactcgccgagggtgaggtccccaccgcactcgctctcgactccggcgtgaccgtggagccggcgggtgacggagtcctgctgcgcggtgaggccgggccggccgtgggtgccgaagccggggtcgccgtgctcgtccgtgtcgcgggggaaggtgatccggccgtcgagagctgggcgctcgtcgaggcggacgatccgggtctgcacatcgaaccgctcgagaccatcgacgcctcccgcgcggtggcccgcgtccgcctcgacggcgcgacggtcccggccgaccgggtcgcgaccgtcccggccggcttcgtgcgcgacctcaccgccggtctcgccgccgcggagctggccggtctcgccggttgggcgctgaccaccgccgtcgagtacgcgaagatccgcgagcagttcggaaaaccgatcggttcgttccaggccgtcaagcacatctgtgccgaaatgctctgccgcaccgagaagatccgggccatggcctgggatgctgcggtcaccgtcgacgcgcagcccgacgaactgccgatcgccgcggctgccgccgtggcggtcgcactcgatgccgcggtgcagaccgccaaggatgcgatccaggtgctcggcggcatcgggttcacgtgggaacacgacgcgcacttctatcttcgccgtgcggtcgccacccgccaggtgctcggtggttcgaccgtgtggcgttcgcggctgacgaccctggtccgcgcaggcgcacgtcgtcacctcggtatcgacctgtccgatcacgaggaggagcgcgcacggatccgtgcggaagtcgagaagatcgccgccgcaccggaatccgagcgccgcgtcgccctcgccgagtcgggtctgctcgcgccgcactggccgcagccgtacggtcgcggagccggtgccgccgaacagctcgtcgtccaggaggagctcgccgccgccggtatcgaacgtcccgatctcgtgatcggctggtgggcggttccgactatcctcgaacacggaacacccgagcagatcgagcgtttcgtgatgcccaccctgcgcggcgatgtggtgtggtgccagctcttctccgagcccggcgccggctcggacctcgcggcgctgcgcacgagcgcggagaaggccgacggcggatgggtgctgcgcgggcagaaggtgtggacctccctcgcgcagcaggcggactgggcgatctgcctcgcccgcaccgaccgcgacgtccccaagcacaagggcatcacctatttcctcgtcgacatgaagtcggcgggcatcacgatctcgccgctgcgcgagatcaccggcgacgcgttgttcaacgaggtcttcctcgattcggtcttcgtgccggacgactgcgtggtcggcaatctcggtgacggctggaagctggcccgcacgactctcgccaacgagcgtgtcgcgatgggcggcaagtcgtcgctggggcagagcatcgaggaactgctcgaactgtcgacccccggtgatcccgtcgcagaggaccgcatcgcgacgcagatcggcgaggcgaccgtcggttcgctcctggatctgcgggcgaccctcgcgcagctcgaaggtcaggatccgggcgccgcgtccagcgtccgcaagctcatcggtgtgcggcagcggcaggacaccgccgagctcgccatggatctcgcgggcgaggccggctgggtggaaggtccgctcacccgggagttcctcaacacccggtgcctgacgatcgccggcgggaccgagcagatcctgctcaccgtggcggccgagcggctgctgggcctgccgcggggttga

SEQ ID NO: 10 - DNA, 2190 bp, Rhodococcus rhodochrous
  fadE34#2
atgactctgggattgagcgacgaggaccgcgaactccgcgactccgtgcgcggctgggcggcacgacacgccacacccgacgtgatccgcacggccgtcgaagcgaagacggaagcccgcccgacgtactggagctcgttcgccgaactcggcatgctgggattgcacctgcccgaagaggtcggaggcgccggtttcggtctgctcgaaacggcgatcgtcgcagaggaactcggacgggccatggtgcccggcccgttccttccgaccgtgatcgtgtccgcggtcctcgacgaggccggccgtcgcagcgaactcgacgggctcgcggacggttcgctgttcggtgcggtcgccctgcagccgggggacctgcgcgtggagcgcgacggcgattccgtcacgctctcgggaacctccggtgtcgctctcggcggccaggtcgcggatgtcttcctgctcgcggccgacgacggtggtgagcgggtattcgtcgtcgtgacccgtgaccgggtcgaggtcacgaacctgcccagctacgacgtgatccgccgcaacgccgagatcaccgtgagtgccgtgccgctgtccgacggggacgtgctggagtcggatccgcatcggatcgtcgatatcgccgcgaccttgttcgccgccgaagccgccggtctcgcggactgggccaccaccaccgccgcggactatgcgcgggtccgcaagcagttcggccgcgtcatcggacagttccagggtgtcaagcacaccgtcgcccggatgctctgcctcaccgaacaggcgcgggtcgtggcctgggacgccgcgcgagcgcggcgcgaggacgtgccggacgacgaggcgtcgctggccgtggcggtcgccgcgtccatcgcccccgaggccgccttccaggtcaccaagaactgcatccaggtgctcggcggtatcggctacacctgggagcacgacgcccacctgtacatgcgccgcgcccagtcgctccgaatcctgctcggctccacggcgtcctggcggcgccgggtcgcccacctcacgctcggcggtgcccgccgcgtgctgagcgtcgatctgccgcccgaggcggaacggatccgcgccgacgtccgtgccgaactcgagccggcgaagtcgctggagaacgcagcgcggaaggcgtatctggcggagaagggttacaccgctccccatctgcccgaaccgtggggcaaggccgccgacgccgtcacgcaactcgtcgtcgccgaggaactgcgcgccgccgaactcgaaccgcacgacatgatcatcggcaactgggtggtgccgaccctcatcgcgcacggcagtaccgagcagatcgagcgattcgtcccgcagtcgctgcgcggggatctcgtgtggtgtcagctcttctccgaacccggcgccggatccgacctcgcgggcctgtccaccaaggccgtcaaggtggacggcggatggaggctcgacggccagaaggtgtggacgtcgatggcacgggtcgcggattggggcatctgcctcgcccgcaccgacgcggaagcgcccaaacacaaaggcctgtcctacttcctgatcgacatcaggaacaccgagggtctcgacatccggccgctgcgagagatcaccggcgaagccctgttcaacgaggtgttcctcgacggcgtgttcgtgcccgacgagtgcctcgtcggcgagcccggggacggatggaagctcgcccgtaccaccctcgcgaacgaacgcgtctccctctcgcacgattcgactttcggtgccggctgcgagactctcatagcgctcgcgaacggtatgcccggtggaccggacgacgaacaactcaccgtcctcggcaaggttctcggcgatgccgcgtccggtggcctcatgggtctgcgtaccgctctacggtccctggccggcgcacagccgggtgccgagtcctccgtcgccaagctcctcggcgtcgagcacctccagcaggtctgggagaccgcgatggactgggccggtactgcgtcgttgctcgacgaccaggaccgaacttcggcgacccacatgttcctcaacgtgcagtgcatgtccatcgccggtgggacgaccaacgtccagctgaacatcatcggtgagcggcttctcggcctgccccgcgatcccgaacccggaaagtga

SEQ ID NO: 11 - DNA, 1185 bp, Rhodococcus rhodochrous
  fadE26
gtggacatctcctacacccccgggcaacaagccctccgcgaggaattgcgggcctatttcgcacagatcatgacccccgagcgccgcgaggcgctcgcggccacgaccggggagtacggctccggcaacgtgtaccgcgaggtcgtgcagcagatgggcaaggacggctggctcaccctcgggtggcccgaggaatacggcggccagaaccgttccgcgatggaccaattgatcttcaccgacgaggcggccatcgccggcgcgcccgtcccgttcctcaccatcgactcggtcgcgccgacgatcatgcactacggcacggacgagcagaaggagttcttcctcccccgcatctccgcgggagaactgcacttctcgatcggctattccgaacccggcgccggcaccgacctcgcctcgctgcgcaccaccgccgtgcgcgacggcgacgagtgggtcatcaacgggcagaagatgtggacgagcctgatcgcctacgccgactacgtctggctcgccgcgcgcaccaacccggatgtcaagaagcacaaggggatcagcgtcttcatcgtgccgaccgacgctcccggcttctcgtacacccccgtgcacaccatggccggccccgacacgagcgccacctactaccaggacgtgcgcgtcccggcgtccgcgctcgtcggtgaggtcgacggcggctgggcgctcatcaccaaccagctcaatcacgagcgggtcgcactcacctccgccggtcccgtgcgcaccgcgctgaccgaggtccggcgctgggcgcaggagacgcacctgcccgacggacgacgggtgatcgaccaggaatgggtgcagatcaacctggcacgcgtccatgccaaggccgaatacctgcagctgatgaactgggacatcgcctcgagcgccggcacgaccccgctcggtccggaggccgcctcggccaacaaggtgttcggcaccgaattcgcgaccgaggcctaccggttgctcatggaggtcctcggacccgcggcgacggtacggcagaactcggccggcgcactgctccgcggccggatcgaacgcatgcaccgcagttccctcatcctcaccttcggtggcggcaccaacgaggtccagcgcgacatcatcgcgatgaccgctctcggccagccgcccgccaagcgttag

SEQ ID NO: 12 - DNA, 2130 bp, Mycobacterium neoaurum
  fadE34
gtgtctgtgctgtccgtcccgaccgatacatcggatgaggccgcggcccgtgaactggtcagagactgggttccgagctctgggtcgatcaccgcgatccgcaacgtcgaactcggcgatccgcaggcctggcgcacgccgtttgccggcttcgccgaactaggggtattcggcgtcgcggtgcccgaggagtacggcggggccggcagcacggtggcggatctgctcgcgatgatcgacgaggcggccgccggcctgatcccgggacccgtcgcggggaccgcacttgccaccctcgtcgccgatgatccggccgtcctggaggcgttggccaccggggagcgcagcgccgggatcgccatgacgtccgacatcacggtcgattccggtaccgccaccggcaccgcgccccacgtgctgggtgccgatcccggcggggtcctcatcctgcctgccgggcagcattggatcctggtggacgcgagttccgacggggtgaccatcgacccgctggaggccaccgacttctcccgaccgctggcccgggtgacgctgacatcggcaccggcgcagcagctgaatgcctcggcgcagcgggtcaccgacctgatggcgactgtgctggcggccgagctggccgggttgtcgcgctggctgctcaacaccgccaacgagtacgccaaggtgcgcgaacagttcggcaagccgatcggcagcttccaggccgtcaaacacatgtgcgcggagatgctgctgcgtagccagcaggtcaccgtcgccgccgccgacgcgatcgcggccgctgccggtgacgacgccgaccagctgtccgtcgccgcggcggtggcggcggccatcggtatcgacgccgcgaagctgaacgcgcgcgactgcatccaggtgctcggcgggatcggcatcacctgggagcacgatgcgcacctgtacctgcgtcgggcatatgcgaacgcgcagttcctcggtggccggtcgcgttggttgcgtcgcgtcgtcgaactgacccgtgccggcgtgcgccgcgaactgcacgtcgacaccgctgatgccgatgccatccgtcccgagatcgccgcggccgccgcccgcatcgccgcgctgcccgaggaccaacgagggcgggcactcgccgaatccgggctgctggccccgcattggccgacgccgtacgggcgggacgcgaccccggccgaacagttggtgatcgacgaggaactggcggctgccgaggtggcgcgccccgatatctcgatcggctggtgggccgctccgacgatccttgccgccggtacgcccgaacagatcgatcggttcatccccggcaccctcaacggcgacatcttctggtgccagctgttctccgagcccggcgcggggtcggatctggcggcgttgcgcaccaaggccgttcgtgtggagaaggatggccgcactggctggtctctgaccggacagaaggtgtggacctccaacgcgcaccgcgccaactggggcatctgcctggcccggaccaacccggacgctccgaaacacaagggcatctcctatttcctggtcgatatgagctcaccgggtatcgatatccggccgctgcgcgagatcaccggtgaggccctgttcaacgaggtcttcttcgatgacctgttcgttcccgacgactgcgtggtcggtgaggtggacggtggctggccgctggcccgtaccacgctggccaacgagcgcgtcgccatcgccaccggcggggcactggacaagggcatggagcatctgcttgccgtgatcggtgaccgggagctcgacggcgccgaggccgatcggctcggtgccctgatcaccctggcccaggtcggttcgctgctggatcagctcatcgcgcggatggcgttgggcggcaatgatcctggtgctccgtcgagcgtgcgcaagctgatcggcgtgcgttatcgacaggggttggccgaggcggcgatggagttccaggacggtggcggcatcgtcgactcgcccgatgtccggtacttcctcaacacccgctgcttgagcatcgccgggggcaccgagcagatcctgctcaccctcgccggtgagcggctgctggggttgccgcgctag

SEQ ID NO: 13 - protein, 394 aa, Rhodococcus rhodochrous
  kshA1
VSLGTSEQSEIREIVAGSAPARFARGWHCLGLAKDFKDGKPHSVHAFGTKLVVWADSNDEIRILDAYCRHMGGDLSQGTVKGDEIACPFHDWRWGGNGRCKNIPYARRVPPIAKTRAWHTLDQDGLLFVWHDPQGNPPPADVTIPRIAGATSDEWTDWVWYTTEVDTNCREIIDNIVDMAHFFYVHYSFPVYFKNVFEGHVASQFMRGQAREDTRPHANGQPKMIGSRSDASYFGPSFMIDDLVYEYEGYDVESVLINCHYPVSQDKFVLMYGMIVKKSDRLEGEKALQTAQQFGNFIAKGFEQDIEIWRNKTRIDNPLLCEEDGPVYQLRRWYEQFYVDVEDVAPEMTDRFEFEMDTTRPVAAWMKEVEANIARKAALDTETRSAPEQSTTAG

SEQ ID NO: 14 - protein, 399 aa, Rhodococcus rhodochrous
  kshA2
VGSTDTEDQVRTIDVGTPPERYARGWHCLGLVRDFADGKPHQVDAFGTSLVVFAGEDGKLNVLDAYCRHMGGNLAQGSVKGNTIACPFHDWRWRGDGKCAEIPYARRVPPLARTRTWPVAEVSGQLFVWHDPQGSKPPAELAVPEVPTYGDPGWTDWVWNSIEVTGSHCREIVDNVVDMAHFFYVHYGMPTYFRNVFEGHTATQVMRSLPRADAVGVSQATNYSAESRSDATYYGPSYMIDKLWSAGRDPESTPNIYLINCHYPISPTSFRLQYGVMVERPEGVPPEQAEQIAQAVAQGVAIGFEQDVEIWKNKSRIDNPLLCEEDGPVYQLRRWYEQFYVDVEDIRPEMVNRFEYEIDTTRALTSWQAEVDENVAAGRSAFAPNLTRAREAASAESGS

SEQ ID NO: 15 - protein, 382 aa, Rhodococcus rhodochrous
  kshA3
MAQIREIDVGEVRTRFARGWHCLGLSRTFKDGKPHAVEAFGTKLVVWADSNGEPKVLDAYCRHMGGDLSQGEIKGDSVACPFHDWRWGGNGKCTDIPYARRVPPLARTRSWITMEKHGQLFVWNDPEGNTPPPEVTIPEIEQYGSDEWTDWTWNQIRIEGSNCREIIDNVVDMAHFFYIHYAFPTFFKNVFEGHIAEQYLNTRGRPDKGMATQYGLESTLESYAAYYGPSYMINPLKNNYGGYQTESVLINCHYPITHDSFMLQYGIIVKKPQGMSPEQSDVLAAKLTEGVGEGFLQDVEIWKNKTKIENPLLCEEDGPVYQLRRWYEQFYVDVADVTEKMTGRFEFEVDTAKANEAWEKEVAENLERKKREEEQGKQEAEV

SEQ ID NO: 16 - protein, 378 aa, Rhodococcus rhodochrous
  kshA4
MTVPQERIEIRNIDPGTNPTRFARGWHCIGLAKDFRDGKPHQVKVFGTDLVVFADTAGKLHVLDAFCRHMGGNLARGEIKGDTIACPFHDWRWNGQGRCEAVPYARRTPKLGRTKAWTTMERNGVLFVWHCPQGSEPTPELAIPEIEGYEDGQWSDWTWTTIHVEGSHCREIVDNVVDMAHFFYVHFQMPEYFKNVFDGHIAGQHMRSYGRDDIKTGVQMDLPEAQTISDAFYYGPSFMLDTIYTVSEGTTIESKLINCHYPVTNNSFVLQFGTIVKKIEGMSEEQAAEMATMFTDGLEEQFAQDIEIWKHKSRIENPLLTEEDGPVYQLRRWYNQFYVDLEDVTPDMTQRFEFEVDTSRALESWHKEVEENLAGTAE

SEQ ID NO: 17 - protein, 390 aa, Rhodococcus rhodochrous
  kshA5
MSIDTARSGSDDDVEIREIQAAAAPTRFARGWHCLGLLRDFQDGKPHSIEAFGTKLVVFADSKGQLNVLDAYCRHMGGDLSRGEVKGDSIACPFHDWRWNGKGKCTDIPYARRVPPIAKTRAWTTLERNGQLYVWNDPQGNPPPEDVTIPEIAGYGTDEWTDWSWKSLRIKGSHCREIVDNVVDMAHFFYIHYSFPRYFKNVFEGHTATQYMHSTGREDVISGTNYDDPNAELRSEATYFGPSYMIDWLESDANGQTIETILINCHYPVSNNEFVLQYGAIVKKLPGVSDEIAAGMAEQFAEGVQLGFEQDVEIWKNKAPIDNPLLSEEDGPVYQLRRWYQQFYVDVEDITEDMTKRFEFEIDTTRAVASWQKEVAENLAKQAEGSTATP

SEQ ID NO: 18 - protein, 511 aa, Rhodococcus rhodochrous
  kstD1
MAEWAEECDVLVVGSGAGGCCGAYTAAREGLSVILVEASEYFGGTTAYSGGGGVWFPTNAVLQRAGDDDTIEDALTYYHAVVGDRTPHELQEAYVRGGAPLIDYLESDDDLEFMVYPWPDYFGKAPKARAQGRHIVPSPLPIAGDPELNESIRGPLGRERIGEPLPDMLIGGRALVGRFLIALRKYPNVDLYRNTPLEELIVEDGVVVGAVVGNDGERRAIRARKGVVLAAGGFDQNDEMRGKYGVPGAARDSMGPWSNLGKAHEAGIAVGADVDLMDQAWWSPGLTHPDGRSAFALCFTGGIFVDQDGARFTNEYAPYDRLGRDVIARMERGEMTLPFWMIYDDRNGEAPPVGATNVPLVETEKYVDAGLWKTADTLEELAGQIGVPAESLKATVARWNELAAKGVDEDFGRGDEPYDLAFTGGGSALVPIEQGPFHAAQFGISDLGTKGGLRTDTVGRVLDSEGAPIPGLYAAGNTMAAPSGTVYPGGGNPIGASALFAHLSVMDAAGR

SEQ ID NO: 19 - protein, 570 aa, Rhodococcus rhodochrous
  kstD2
MAKTPVPAVTTARDTTVDLLVIGSGTGMAAALTAHEAGLSALIVEKSAYVGGSTARSGGAFWVPANPVLTAAGSGDTIERGHTYVRTVVDGTAPVERGEAFVDNGVATIEMLQRTTPMKLFWAEGYSDYHPELAGGSAVGRSCECLPLDLSVLGEERGRLRPGLMEASLPMPTTGADYKWMNLMLRVPHKGFPRIFKRLAQGVAGLAVKREYVAGGQAIAAGLFAGVLKAGVPVWTETSLVRLLTDGDRVTGAVVEQNGREVTVTARRGVVLAAGGFDHDMEMRRKFQSERLLDHESLGAETNTGDAIKAAQEVGADLALMDQAWWFPAVAPTRTGKPPMVMLAERSLPGSFIVDQTGRRFTNESSDYMSFGQLVLERERAGDPIESMWIVFDQKYRNSYVFAAGVFPRQPLPEAWYEAGIAHRGTTAAELAASMGVPVDTFAATFDRFNEDAAAGTDSEFGRGGSAYDRYYGDPTVQPNPNLRPLTHGPLYAVKMTLSDLGTCGGVRADERARVLREDGSPIAGLYAIGNTAANAFGHRYPGAGATIGQGLVFGYIAARDAASSDAPVA

SEQ ID NO: 20 - protein, 570 aa, Rhodococcus rhodochrous
  kstD3
MTKQEYDIVVVGSGAGGMTAAITAARKGADVVLIEKAPRYGGSSARSGGGVWIPNNEALKAAGVDDTPEEARKYLHSIIGDDVPAEKIDTYIDRGPEMLSFVLKNSALELQWVPGYSDYYPEAPGGRPGGRSVEPTPFDGRRLGEDLALLEPDYARAPKNFVITQADYKWLNLLMRNPRGPIRAMRVGARFVWANITKKHLLVRGQALMAGLRIGLRDAGVPLLLETALTDLVVEGGAVRGVKVVANGETRVIRARKGVIIASGGFEHNAEMRAQYQRQPIGTEWTVGAKANTGDGIRAGQKLGAAVDFMDDAWWGPSFTLTGGPWFALSERSLPGCLMVNAAGKRFVNESAPYVEATHAMYGGKHGRGEGPGENIPSWLILDQRYRDRYTFAGITPRTPFPRRWLEAGVLVKAGSVAELAEKIGVPADALTETVQRFNGFARAGKDEDFGRGESHYDHYYGDPRNKPNPSLGVVDKAPFYAFKVVPGDLGTKGGLVTDVHGRVVREDGSVIDGLYATGNASSPVMGHTYAGPGATIGPAMTFGYLAALDILDRTGDERTEELRESADTV

SEQ ID NO: 21 - protein, 715 aa, Rhodococcus rhodochrous
  fadE34
VSIATTEEQRAVQASVQAWSRAVDPMSTIRRAGDATWRDGWSSLAELGIFGVAVPEEAGGLGATAVDLAVMLEQAAHELAPGPVLTTAVAALVFGRAGETVAKTAERLAEGEVPTALALDSGVTVEPAGDGVLLRGEAGPAVGAEAGVAVLVRVAGEGDPAVESWALVEADDPGLHIEPLETIDASRAVARVRLDGATVPADRVATVPAGFVRDLTAGLAAAELAGLAGWALTTAVEYAKIREQFGKPIGSFQAVKHICAEMLCRTEKIRAMAWDAAVTVDAQPDELPIAAAAAVAVALDAAVQTAKDAIQVLGGIGFTWEHDAHFYLRRAVATRQVLGGSTVWRSRLTTLVRAGARRHLGIDLSDHEEERARIRAEVEKIAAAPESERRVALAESGLLAPHWPQPYGRGAGAAEQLVVQEELAAAGIERPDLVIGWWAVPTILEHGTPEQIERFVMPTLRGDVVWCQLFSEPGAGSDLAALRTSAEKADGGWVLRGQKVWTSLAQQADWAICLARTDRDVPKHKGITYFLVDMKSAGITISPLREITGDALFNEVFLDSVFVPDDCVVGNLGDGWKLARTTLANERVAMGGKSSLGQSIEELLELSTPGDPVAEDRIATQIGEATVGSLLDLRATLAQLEGQDPGAASSVRKLIGVRQRQDTAELAMDLAGEAGWVEGPLTREFLNTRCLTIAGGTEQILLTVAAERLLGLPRG

SEQ ID NO: 22 - protein, 729 aa, Rhodococcus rhodochrous
  fadE34#2
MTLGLSDEDRELRDSVRGWAARHATPDVIRTAVEAKTEARPTYWSSFAELGMLGLHLPEEVGGAGFGLLETAIVAEELGRAMVPGPFLPTVIVSAVLDEAGRRSELDGLADGSLFGAVALQPGDLRVERDGDSVTLSGTSGVALGGQVADVFLLAADDGGERVFVVVTRDRVEVTNLPSYDVIRRNAEITVSAVPLSDGDVLESDPHRIVDIAATLFAAEAAGLADWATTTAADYARVRKQFGRVIGQFQGVKHTVARMLCLTEQARVVAWDAARARREDVPDDEASLAVAVAASIAPEAAFQVTKNCIQVLGGIGYTWEHDAHLYMRRAQSLRILLGSTASWRRRVAHLTLGGARRVLSVDLPPEAERIRADVRAELEPAKSLENAARKAYLAEKGYTAPHLPEPWGKAADAVTQLVVAEELRAAELEPHDMIIGNWVVPTLIAHGSTEQIERFVPQSLRGDLVWCQLFSEPGAGSDLAGLSTKAVKVDGGWRLDGQKVWTSMARVADWGICLARTDAEAPKHKGLSYFLIDIRNTEGLDIRPLREITGEALFNEVFLDGVFVPDECLVGEPGDGWKLARTTLANERVSLSHDSTFGAGCETLIALANGMPGGPDDEQLTVLGKVLGDAASGGLMGLRTALRSLAGAQPGAESSVAKLLGVEHLQQVWETAMDWAGTASLLDDQDRTSATHMFLNVQCMSIAGGTTNVQLNIIGERLLGLPRDPEPGK

SEQ ID NO: 23 - protein, 394 aa, Rhodococcus rhodochrous
  fadE26
MDISYTPGQQALREELRAYFAQIMTPERREALAATTGEYGSGNVYREVVQQMGKDGWLTLGWPEEYGGQNRSAMDQLIFTDEAAIAGAPVPFLTIDSVAPTIMHYGTDEQKEFFLPRISAGELHFSIGYSEPGAGTDLASLRTTAVRDGDEWVINGQKMWTSLIAYADYVWLAARTNPDVKKHKGISVFIVPTDAPGFSYTPVHTMAGPDTSATYYQDVRVPASALVGEVDGGWALITNQLNHERVALTSAGPVRTALTEVRRWAQETHLPDGRRVIDQEWVQINLARVHAKAEYLQLMNWDIASSAGTTPLGPEAASANKVFGTEFATEAYRLLMEVLGPAATVRQNSAGALLRGRIERMHRSSLILTFGGGTNEVQRDIIAMTALGQPPAKR

SEQ ID NO: 24 - protein, 709 aa, Mycobacterium neoaurum
  fadE34
VSVLSVPTDTSDEAAARELVRDWVPSSGSITAIRNVELGDPQAWRTPFAGFAELGVFGVAVPEEYGGAGSTVADLLAMIDEAAAGLIPGPVAGTALATLVADDPAVLEALATGERSAGIAMTSDITVDSGTATGTAPHVLGADPGGVLILPAGQHWILVDASSDGVTIDPLEATDFSRPLARVTLTSAPAQQLNASAQRVTDLMATVLAAELAGLSRWLLNTANEYAKVREQFGKPIGSFQAVKHMCAEMLLRSQQVTVAAADAIAAAAGDDADQLSVAAAVAAAIGIDAAKLNARDCIQVLGGIGITWEHDAHLYLRRAYANAQFLGGRSRWLRRVVELTRAGVRRELHVDTADADAIRPEIAAAAARIAALPEDQRGRALAESGLLAPHWPTPYGRDATPAEQLVIDEELAAAEVARPDISIGWWAAPTILAAGTPEQIDRFIPGTLNGDIFWCQLFSEPGAGSDLAALRTKAVRVEKDGRTGWSLTGQKVWTSNAHRANWGICLARTNPDAPKHKGISYFLVDMSSPGIDIRPLREITGEALFNEVFFDDLFVPDDCVVGEVDGGWPLARTTLANERVAIATGGALDKGMEHLLAVIGDRELDGAEADRLGALITLAQVGSLLDQLIARMALGGNDPGAPSSVRKLIGVRYRQGLAEAAMEFQDGGGIVDSPDVRYFLNTRCLSIAGGTEQILLTLAGERLLGLPR

SEQ ID NO: 25 - DNA, 20 bp, Artificial Sequence
  kstD1 forward primer
tggcagcagaactcgccggg

SEQ ID NO: 26 - DNA, 22 bp, Artificial Sequence
  kstD1 reverse primer
ccggaacgacaccgatgcgccg

SEQ ID NO: 27 - DNA, 24 bp, Artificial Sequence
  kstD2 forward primer
ctacagcgactaccaccccgattt

SEQ ID NO: 28 - DNA, 23 bp, Artificial Sequence
  kstD2 reverse primer
ctgttgcggtacttctggtcgaa

SEQ ID NO: 29 - DNA, 21 bp, Artificial Sequence
  kstD3 forward primer
cgacctgtcacagggcgagat

SEQ ID NO: 30 - DNA, 23 bp, Artificial Sequence
  kstD3 reverse primer
ggaccaccttgaacgcgtagaac

SEQ ID NO: 31 - DNA, 32 bp, Artificial Sequence
  FadE34-UP forward primer
gcgataagatcttggtggcggatgacgtcgag

SEQ ID NO: 32 - DNA, 31 bp, Artificial Sequence
  FadE34-UP reverse primer
gcgatatctagaggcccgctgctcctcggtc

SEQ ID NO: 33 - DNA, 30 bp, Artificial Sequence
  FadE34-DOWN forward primer
gcgatatctagaatcgccggcgggaccgag

SEQ ID NO: 34 - DNA, 30 bp, Artificial Sequence
  FadE34-DOWN reverse primer
gcgataaagcttgcaggaacttccgcttct

SEQ ID NO: 35 - DNA, 31 bp, Artificial Sequence
  FadE34#2-UP forward primer
gcgataagatctccttctgctggtcgatctg

SEQ ID NO: 36 - DNA, 32 bp, Artificial Sequence
  FadE34#2-UP reverse primer
cgctattctagagagttcggcgaacgagctcc

SEQ ID NO: 37 - DNA, 38 bp, Artificial Sequence
  FadE34#2-DOWN forward primer
gcgatatctagattgctcgacgaccaggaccgaacttc

SEQ ID NO: 38 - DNA, 33 bp, Artificial Sequence
  FadE34#2-DOWN reverse primer
cgctataagcttagctgtgcggtggcgccgctg

SEQ ID NO: 39 - DNA, 25 bp, Artificial Sequence
  Flanking_fadE34 forward primer
gaacgcgagcgcggcgatgacctct

SEQ ID NO: 40 - DNA, 25 bp, Artificial Sequence
  Flanking_fadE34 reverse primer
ggtccagctgaagccgggatccttg

SEQ ID NO: 41 - DNA, 30 bp, Artificial Sequence
  Flanking_fadE34#2 forward primer
gaggtcgccgaactcgccggtgtcgccatc

SEQ ID NO: 42 - DNA, 30 bp, Artificial Sequence
  Flanking_fadE34#2 reverse primer
gcgtgcacctgttcgcggtcggtgacatcc

SEQ ID NO: 43 - DNA, 32 bp, Artificial Sequence
  kshA5-complem forward primer
gcgataggatccggcccggattgtcgctgatg

SEQ ID NO: 44 - DNA, 29 bp, Artificial Sequence
  kshA5-complem reverse primer
cgctataagcttgatcacgtgcagcatgc

SEQ ID NO: 45 - DNA, 33 bp, Artificial Sequence
  FadE34_Mneo-UP forward primer
gcgataggatccgacaccgacttcctgctgttg

SEQ ID NO: 46 - DNA, 32 bp, Artificial Sequence
  FadE34_Mneo-UP reverse primer
cgctattctagaccgatgtccggtacttcctc

SEQ ID NO: 47 - DNA, 32 bp, Artificial Sequence
  FadE34_Mneo-DOWN forward primer
gcgatatctagagatcgccgagttcgacgttg

SEQ ID NO: 48 - DNA, 32 bp, Artificial Sequence
  FadE34_Mneo-DOWN reverse primer
cgctataagcttgtgacgatcaccgcgaactc

SEQ ID NO: 49 - DNA, 21 bp, Artificial Sequence
  FadE34_Mneo forward primer
agattcggtgcagaccgattg

SEQ ID NO: 50 - DNA, 19 bp, Artificial Sequence
  FadE34_Mneo reverse primer
aagctgcatgcggatccac